(19) United States
(12) Patent Application Publication
(10) Pub. No.: US 2024/0058103 A1
(43) Pub. Date: Feb. 22, 2024

(54) NANOFIBROUS TISSUE ENGINEERING MATRICES WITH IMPROVED CLINICAL HANDLING PROPERTIES FOR PERIODONTAL AND CRANIOFACIAL REGENERATION AND METHODS OF MAKING THE SAME

(71) Applicant: REGENTS OF THE UNIVERSITY OF MICHIGAN, Ann Arbor, MI (US)

(72) Inventors: William Benton Swanson, Ann Arbor, MI (US); Seth Woodbury, Ann Arbor, MI (US); Yuji Mishina, Ann Arbor, MI (US)

(21) Appl. No.: 18/141,138

(22) Filed: Apr. 28, 2023

Related U.S. Application Data
(60) Provisional application No. 63/336,208, filed on Apr. 28, 2022.

(51) Int. Cl. A61C 8/02; A61L 31/04; A61L 31/14

(52) U.S. Cl. CPC A61C 8/0006; A61L 31/041; A61L 31/148; A61L 2400/12; A61L 2430/34

(57) ABSTRACT

The disclosure relates generally to a tissue engineering matrix with improved clinical handling properties, designed for periodontal and craniofacial regeneration applications and methods of manufacturing the same. In one application, these matrices are fabricated as surgical membranes which serve not only as a protective barrier but also to induce regeneration through controlled release of inductive substances, facilitate regeneration through the tissue integration, and define and maintain dimensional stability in horizontal and/or vertical defects. In another application, these matrices are fabricated as macroporous scaffolds, and the novel chemistry serves to deliver a three-dimensional environment capable of facilitating tissue ingrowth, vascularization, extracellular matrix deposition and remodeling, and tissue regeneration.

### CROSS-REFERENCE TO RELATED APPLICATIONS

This application claims the benefit of and priority to U.S. Provisional Patent Application No. 63/336,208, filed on Apr. 28, 2022, the entirety of which is hereby incorporated by reference.

### GOVERNMENT RIGHTS

This invention was made with U.S. government support under grant number F30DE029359 and R01DE DE027662 awarded by the National Institutes of Health. The U.S. government has certain rights in the invention.

### FIELD

The present technology relates generally to a tissue engineering matrix having at least two biodegradable polymers forming interpenetrating polymer network with improved clinical handling properties for periodontal and craniofacial regeneration applications, and a method of preparing the same.

### INTRODUCTION

The periodontal complex involves the hard and soft tissues which support dentition. The goal of tissue engineering and regenerative medicine is to repair and replace tissues which are damaged in the course of disease, with the goal of restoring physiologic function. In the case of periodontal tissues, the field of periodontology has made significant progress to date in translating periodontal regeneration techniques to the patient care setting. While significant progress has been made, large scale clinical trials show significant heterogeneity in clinical outcomes, largely attributed to surgeon skill, patient selection, and patient compliance.

Periodontal disease involves the loss of tooth-supporting structures; its hallmark feature is loss of alveolar bone. Along with alveolar bone loss is loss of the bone-integrated support structure—the periodontal ligament (PDL), and tooth-integrated cementum. Periodontitis is the 11^{th} most common disease worldwide and is identified by the US Surgeon General's Report on Oral Health as a critical challenge to human health and disease. Therefore, periodontal disease represents a significant healthcare burden, and developing regenerative treatments is a meaningful and important opportunity.

Scaling and root planning (SRP) remains a gold standard non-surgical treatment for periodontal defects by mechanical removal of disease-causing agents. The primary goal in removing subgingival calculus, biofilm deposits, and diseased cementum is to create a root surface capable of reattachment with periodontal tissues. SRP along with oral hygiene instruction for home care and patient compliance results in favorable clinical outcomes in a non-surgical approach to treatment. The effectiveness of SRP becomes limited by instrument limitations, where curet efficiency decreases below 3.73 mm pocket depth and reaches a limit of about 6 mm depth. Beyond a critical probing depth of 5.4 mm, open flap debridement, a form of surgical periodontal therapy, may be preferred.

The goals of periodontal surgical therapy are to treat periodontal disease or modify the morphological status of the periodontium, enabled by surgical access to deeper defects. Periodontal regeneration aims to facilitate the formation of new bone, cementum, and a functionally oriented periodontal ligament at a site deprived of its initial attachment apparatus.

Broadly, periodontal regeneration can be described in terms of guided tissue regeneration (GTR) and guided bone regeneration (GBR). Guided bone regeneration (GBR) seeks to fulfill the specific goal of bone regeneration in preparation for dental implant placement, without implicit concern for the PDL. Examples of these procedures may include alveolar ridge reconstruction, sinus floor augmentation, and regeneration of preimplant osseous defects. Membranes serve as a protective barrier to isolate a defect site and facilitate alveolar bone remodeling and regeneration. Guided tissue regeneration (GTR) seeks to reconstitute loss periodontal structures through different tissue responses, allowing for regeneration of the periodontal attachment apparatus which includes bone, PDL, and cementum. Clinical indications of GTR were summarized by Reynolds et al. (M. A. Reynolds, Periodontal regeneration—intrabony defects: a consensus report from the AAP Regeneration Workshop, J Periodontol. 2015 February; 86(2 Suppl):S105-7. doi: 10.1902/jop.2015.140378. Epub 2014 Oct. 15.). In addition to barrier membranes described above which allow for selective repopulation of the root surface, growth factors may play a role in both development and regeneration and are a key component of tissue engineering therapies as reported by Avila-Ortiz et al. (Avila-Ortiz, American Academy of Periodontology best evidence consensus statement on the use of biologics in clinical practice, J. Periodontology, Volume 93, Issue 12, December 2022, Pages 1763-1770). In the case of GTR, new attachment involves the regeneration of the principal fibers which compose the periodontal ligament, and their reattachment into newly formed cementum on the root surface. This process, performed in part by periodontal ligament cells, competes with apical migration of the pocket-lining junctional epithelium. Nyman et al were among the first to demonstrate that new periodontal ligament can be established on a previously diseased root surface (S. Nyman, J. Lindhe, T. Karring, H. Rylander, New attachment following surgical treatment of human periodontal disease, Journal of Clinical Periodontology 9(4) (1982) 290-296.). Their seminal study in a human patient demonstrated that cells originating from the periodontal ligament may migrate coronally to a root surface previously exposed to a periodontal pocket to deposit new cementum and attachment fibers, when adequately isolated from the sulcular epithelium with a cellulose acetate membrane. Furthermore, they demonstrated that the formation of new connective tissue attachment is not necessarily accompanied by coronal regeneration of alveolar bone.

Nonresorbable barrier membranes from polytetrafluoroethylene (PTFE) were introduced as the original periodontal membranes. These membranes require a second surgery to remove the membrane. Furthermore, various clinical reports identify early exposure and bacterial colonization as factors leading to regenerative failures. As a result, lower levels of attachment gain are reported. The second generation of surgical membranes for periodontal regeneration are focused on collagen membranes. Collagen is biologically derived, biocompatible, and biodegradable. Several complications such as early membrane degradation, epithelial ingrowth, epithelial downgrowth, and premature loss of material are reported in the literature. Additionally, collagen membranes lack the ability to be sutured, reliably fixed, or maintain dimension within a clinical defect. Collagen membranes are derived from bovine or porcine type I and III collagen, which has been shown in the literature to limit attachment and proliferation of human PDL cells and human osteoblasts, compared to cells plated on culture dishes.

Accordingly, a need exists for new membranes and scaffolds that addresses one or more of the technical issues discussed above and new methods of making the same.

### SUMMARY

The technology of the present disclosure includes biodegradable polymeric materials, biodegradable surgical membranes, macroporous scaffolds, and controlled release drug systems having macroporous scaffolds and drugs embedded in the scaffolds, and methods of making same, that relates to a new tissue engineering matrix with improved clinical handling properties, designed for periodontal and craniofacial regeneration applications. In one application, these matrices are fabricated as surgical membranes which not only serve as a protective barrier but also induce regeneration through controlled release of inductive substances, facilitate regeneration through their tissue integration, and define and maintain dimensional stability in horizontal and/or vertical defects. In another application, these matrices are fabricated as macroporous scaffolds, wherein the novel chemistry of the macroporous scaffolds serves to deliver a three-dimensional environment capable of facilitating tissue ingrowth, vascularization, extracellular matrix deposition and remodeling, and tissue regeneration.

One aspect of the present disclosure provides a method of making a biodegradable polymeric material. The method comprises: reacting a polymer mixture comprising a first polymer and a second polymer, the first polymer being a biodegradable linear polymer and having first polymer chains, the second polymer being a biodegradable polymer having second polymer chains each of which has two or more functional groups, the first and second polymers being present in a weight ratio of between about 50:50 and about 99:1; and a reagent to form coupled second polymer chains in presence of the first polymer chains, thereby forming the biodegradable polymeric material, wherein the reagent is selected from a group of a radical initiator, a catalyst, a crosslinking agent, or a combination thereof, and wherein the biodegradable polymeric material has an interpenetrating polymer network (IPN) or a semi-interpenetrating polymer network (SIPN) between the first and second polymer chains, wherein the coupled second polymer chains are interspersed throughout the first polymer chains.

In embodiments, the second polymer is different from the first polymer. In embodiments, the second polymer is a linear polymer and has two terminus and two functional groups each of which is at one of the two terminus of the second polymer chain. In embodiments, the second polymer is a branch polymer wherein each branch has a terminus and each terminus can include a functional group. In embodiments, the reacting includes extending and/or at least partially crosslinking the second polymer chains with each other through the two or more functional groups. In embodiments, the polymer mixture may comprise a third polymer with two or more functional groups, and the biodegradable polymeric material has the IPN or SIPN between the first, second and third polymer chains. In embodiments, the two or more functional groups of the third polymer can with each other and/or with the two functional groups of the second polymer. In embodiments, the first polymer is at least partially crystallized in the biodegradable polymeric material and has a crystallinity of about 10-100%, or about 20-90%, or about 20-80%, or about 30-70%, or about 40-60%. In embodiments, the first polymer is at least partially phase separated from the second polymer in nanoscale level and forms crystalline nanofibers in the IPN or SIPN.

Another aspect of the present disclosure provides a biodegradable polymeric material comprising: a first polymer and a second polymer provided in a weight ratio of between about 50:50 and about 99:1, wherein the first polymer is a biodegradable linear polymer having first polymer chains and a crystallinity of about 10-100%, about 20-90%, or about 20-80%, or about 30-70%, or about 40-60%, wherein the second polymer is a biodegradable polymer different from the first polymer and having second polymer chains each of which has two or more functional groups, and at least a portion of the second polymer chains are coupled to each other through the functional groups, wherein the biodegradable polymeric material has an IPN or SIPN between the first and second polymer chains, and the second polymer chains are interspersed throughout the first polymer chains. In embodiments, the second polymer may be at least partially crosslinked with each other and may have a crosslinking density of about 0.1-50%, or about 1-20%.

Another aspect of the present disclosure provides a method of making a biodegradable surgical membrane, the method comprising: admixing a polymer mixture and a first organic solvent to form a homogeneous polymer mixture solution, wherein the polymer mixture comprises a first polymer and a second polymer in a weight ratio in a range of about 50:50 and about 99:1, the first polymer being a biodegradable linear polymer having first polymer chains, the second polymer being a biodegradable polymer different from the first polymer and having second polymer chains each of which has two or more functional groups; admixing a reagent solution with the polymer mixture solution to form a polymer-reagent solution, wherein the reagent solution comprises a reagent and a second organic solvent, and the reagent is a radical initiator, a catalyst, a crosslinking agent, or a combination thereof; and reacting the polymer-reagent solution to form coupled second polymer chains in the presence of the first polymer chains, thereby forming the biodegradable surgical membrane, wherein the biodegradable surgical membrane has an IPN or SIPN between the first polymer chains and the coupled second polymer chains. In embodiments, the reacting includes extending and/or at least partially crosslinking the second polymer chains with each other through the two or more functional groups. In embodiments, the polymer mixture solution comprises about 1-30 wt. % of the polymer mixture and about 70-99 wt. % of the first organic solvent by weight of the polymer mixture solution.

Another aspect of the present disclosure provides a biodegradable surgical membrane comprising: a first polymer and a second polymer in a weight ratio of between about 50:50 and about 99:1, the first polymer being a biodegradable linear polymer having first polymer chains and a crystallinity of about 10-100% or about 20-80%; the second polymer being a biodegradable polymer and having second polymer chains each of which has two or more functional groups, wherein at least a portion of the second polymer chains are coupled with each other through the two or more functional groups, wherein the biodegradable surgical membrane has an IPN or SIPN between the first and second polymer chains, and wherein the second polymer chains are interspersed throughout the first polymer chains. In embodiments, the biodegradable surgical membrane may have a biphasic morphology including: a top smooth layer having a porosity no more than about 40% v/v; and a bottom porous layer having a porosity of at least about 60% v/v. In embodiments, the second polymer may be partially crosslinked and may have a crosslinking density of about 0.1-50% or about 1-20%. In embodiments, the second polymer has two functional groups, the biodegradable surgical membrane may comprise a third polymer having third polymer chains each of which has three or more functional groups, the third polymer is present in an amount of about 0.1-40 wt. % or about 1-20 wt. % of the biodegradable surgical membrane, the third polymer may be partially crosslinked with each other and/or with the second polymer chains, the IPN or SIPN may comprise the first, second and third polymer chains interpenetrating with each other, and the second and third polymer chains are interspersed throughout the first polymer chains.

Another aspect of the present disclosure provides a method for providing a dental implant to a subject in need thereof, the method comprising: implanting a periodontal membrane comprising the biodegradable surgical membrane discussed above or elsewhere in the present disclosure to a defective dental site in the subject.

Another aspect of the present disclosure provides a method for making a macroporous tissue engineering scaffold, the method comprising: admixing a polymer mixture comprising a first polymer and a second polymer in a weight ratio between about 50:50 and about 99:1, the first polymer being a biodegradable linear polymer and having first polymer chains; the second polymer being a biodegradable polymer and having second polymer chains each of which includes two or more functional groups; and a first organic solvent to form a polymer mixture solution, wherein the polymer mixture solution comprises about 1-30 wt. % of the polymer mixture by weight of the polymer mixture solution and about 70-99 wt. % of the first organic solvent by weight of the polymer mixture solution; admixing a reagent solution with the polymer mixture solution to form a polymer-reagent solution, wherein the reagent solution comprises a reagent and a second organic solvent and the reagent is selected from a group of a radical initiator, a catalyst, a crosslinking agent, or a combination thereof; combining in a container the polymer-reagent solution and a sugar porogen scaffold template comprising sugar particles; reacting the polymer-reagent solution to form coupled second polymer chains, thereby forming an initial scaffold including the sugar porogen scaffold template; cooling the initial scaffold; and dissolving the sugar particles in the initial scaffold thereby forming the macroporous tissue engineering scaffold, wherein the macroporous tissue engineering scaffold has an IPN or SIPN between the first and second polymer chains, and wherein the second polymer chains are interspersed throughout the first polymer chains.

Another aspect of the present disclosure provides a macroporous tissue engineering scaffold comprising: a first polymer and a second polymer in a weight ratio of between about 50:50 and about 99:1, the first polymer being a biodegradable linear polymer and having first polymer chains, the second polymer being a biodegradable polymer, the second polymer having second polymer chains each of which has two or more functional group, at least a portion of the second polymer being coupled with each other through the two or more functional groups, wherein the macroporous tissue engineering scaffold has an IPN or SIPN between the first and second polymer chains, wherein the second polymer chains are interspersed throughout the first polymer chains, wherein the first polymer is at least partially crystallized and at least partially phase separated from the second polymer, the first polymer has a crystallinity of 20-80%, and the first polymer having crystalline nanostructures homogeneously dispersed in the IPN or SIPN, and wherein the macroporous tissue engineering scaffold has a porosity of about 50-99% v/v or about 90-98% v/v and has macro-pores having an average pore size of about 0.1-1000 μm or about 30-450 μm. In embodiments, the second polymer may be at least partially crosslinked and may have a crosslinking density of about 0.1-50% or about 1-20%.

Another aspect of the present disclosure provides a method for providing a dental implant to a subject in need thereof, the method comprising: implanting the macroporous tissue engineering scaffold discussed herein above or elsewhere in the present disclosure to a defective dental site in the subject.

Another aspect of the present disclosure provides a method of preparing a system comprising a macroporous tissue engineering scaffold and a controlled release composition embedded in the scaffold, the method comprising: admixing a polymer mixture comprising a first polymer and a second polymer in a weight ratio in a range between about 50:50 and about 99:1, the first polymer being a biodegradable linear polymer and having first polymer chains, the second polymer being a biodegradable polymer and having second polymer chains each of which includes two or more functional groups; and a first organic solvent to form a polymer mixture solution having 1-30 wt. % of the first and second polymers and 70-99 wt. % of the first organic solvent by weight of the polymer mixture solution; admixing a reagent solution with the polymer mixture solution in a volume ratio of between about 0.1:100 v/v and about 20:1 v/v, or between about 1:100 v/v and about 10:1 v/v, to form a polymer-reagent solution, the reagent solution comprising a reagent and a second organic solvent and having a concentration of about 0.01-100 mM, about 0.1-50 mM, about 1-10 mM, or about 3 mM of reagent in the reagent solution, wherein the reagent is selected from a group of a radical initiator, a catalyst, a crosslinking agent, or a combination thereof; combining into a container the polymer-reagent solution and a sugar porogen scaffold template, the sugar porogen scaffold template comprising sugar particles and nanoparticles having the controlled release composition, and the nanoparticles attached to a surface of the sugar particles; reacting the polymer-reagent solution in the container to form coupled second polymer chains, thereby forming an initial scaffold including the sugar porogen scaffold template, wherein the reacting includes coupling (including chain-extended and/or at least partially crosslinked) the second polymer chains in a matrix of the first polymer to for an IPN or SIPN between the first and coupled second polymer chains, and wherein the coupled second polymer chains are interspersed throughout the first polymer chains.

Another aspect of the present disclosure provides a system having a macroporous tissue engineering scaffold and a controlled release composition embedded in the macroporous scaffold, the system comprising: the macroporous tissue engineering scaffold comprising: a first polymer and a second polymer in a weight ratio of between about 50:50 and about 99:1, the first polymer being a biodegradable linear polymer having first polymer chains, the second polymer being a biodegradable polymer having second polymer chains each of which has two or more functional groups, at least a portion of the second polymer chains being coupled (such as chain extended and/or at least partially crosslinked) with each other through the two or more functional groups, wherein the macroporous scaffold has an IPN or SIPN between the first and second polymer chains, wherein the second polymer chains are interspersed throughout the first polymer chains, and wherein the first polymer is at least partially crystallized and at least partially phase separated from the coupled second polymer, the first polymer has a crystallinity of about 10-90% or about 20-80%, and the first polymer having crystalline nanostructures homogeneously dispersed in the IPN or SIPN; and the controlled release composition comprising: first nanoparticles comprising a fourth polymer and a first drug substance homogeneously dispersed in the fourth polymer, wherein the first nanoparticles are dispersed in the macroporous scaffold, the nanoparticles have an average particle size of 10-1000 nm, and wherein the macroporous scaffold having the controlled release composition has a porosity of about 50-99% or about 90-98% v/v and has macro-pores having an average pore size of about 0.1-1000 μm or about 30-450 μm.

Another aspect of the present disclosure provides a method for providing a dental implant to a subject in need thereof, the method comprising: implanting the system having a macroporous scaffold and a controlled release composition embedded in a macroporous scaffold disclosed herein above to a defective dental site in the subject.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIGS. 1A and 1B. Surgical membranes (white) are used as a partition in periodontal regeneration to allow selective healing of one compartment of tissue (bone, lower) while excluding epithelial cell infiltration (gingiva, upper). This concept of selective repopulation allows for complete regeneration (as shown in FIG. 1B) of osseous defects (as shown in FIG. 1A).

FIGS. 2A-2B. FIG. 2A shows a scheme of synthesis of poly (L-lactic acid), PLLA, by Sn(Oct)₂-catalyzed ring opening metastasis polymerization (ROMP). FIG. 2B shows 1H NMR of PLLA polymer in CDCl₃. 5.15 ppm represents CH shift, 1.58 ppm represents —CH₃ shift. TMS peak at 0 ppm and CDCl₃ peak at 7.4 ppm.

FIGS. 3A-3B. Bifunctional poly(caprolactone) (PCL) diol is synthesized in the melt by ring opening metastasis polymerization (ROMP), catalyzed by Tin(II) 2-ethylhexanoate, commonly referred to as stannous octoate (Sn(Oct)₂) as shown in FIG. 3A. A diol initiator enables bidirectional polymerization and resulting diol polymer. FIG. 3B shows 1H NMR spectrum of PCL in CDCl₃.

FIGS. 4A-4C. Diacrylation of PCL is performed by end group activation by triethylamine (TEA) followed by subsequent nucleophilic substitution with acryloyl chloride at 0° C. in DCM, as shown in FIG. 4A. FIG. 4B shows 1H NMR analysis of diacrylation reaction in FIG. 5A, observed in CDCl₃. Triacrylation of PCL is performed by end group activation by triethylamine (TEA) followed by subsequent nucleophilic substitution with acryloyl chloride at 0° C. in DCM, as shown in FIG. 4C. A triol (1,3,5-pentanetriol) initiator enables tridirectional polymerization and resulting triol polymer.

FIGS. 5A and 5B. 1H NMR analysis of diacrylation reaction to form the PLGA-DA, as observed in CDCl₃, as shown in FIG. 5A. FIG. 5B shows the scheme of synthesis of PLGA-TA by Sn(Oct)₂-catalyzed ring opening metastasis polymerization (ROMP).

FIG. 6. Schematic overview of IPN or SIPN biomaterial.

FIG. 7. 1H NMR analysis of the PLLA/PCL-DA/PLGA-DA (60/20/20), as observed in CDCl₃.

FIG. 8. STL rendering of plastic molds which are prepared by 3D printing to synthesize and fabricate surgical membranes.

FIG. 9. A photo of an example of a thermosensitive memorized microstructure (TS-MMS) scaffold made from PLLA/PCL-DA/PLGA-DA (60/20/20) material after processing from the sugar sphere porogen method and leaching sugar spheres with water.

FIGS. 10A-10J. Scanning electron micrographs (SEM) are used to visualize nanofibers resulting from thermally induced phase separation (TIPS) as a function of PLLA/PCL-DA ratios of 100/0, 90/10, 80/20, 70/30, 60/40, 50/50, 40/60, 30/70, 20/80, and 0/100, respectively.

FIGS. 11A-11D. SEM micrographs used to observe nanofiber formation as a function of material concentration as cast from solution at 5 wt. %, 10 wt. %, 15 wt. % and 20 wt. %, respectively.

FIGS. 12A-12C. SEM micrographs used to observe the effect of PCL-DA molecular weight on TIPS-nanofiber formation.

FIGS. 13A-13B. Dynamic scanning calorimetry (DSC) for various material compositions illustrates a biphasic profile for TS-MMS materials, compared to monophasic profiles of PLLA and PCL-DA, respectively.

FIGS. 14A-14B. Enthalpy is calculated from DSC (FIG. 14A); enthalpy is directly correlated to the weight percent incorporation of PLLA (FIG. 14B).

FIG. 15. Melting temperature (Tm) is calculated from DSC; PCL-DA imparts a significantly lower Tm to TS-MMS materials compared to PLLA.

FIG. 16. DSC scan of PLLA/PCL-DA TS-MMS scaffold with different amounts of PCL-DA as compared to PLLA and PCL.

FIG. 17. Tm is calculated for scaffold samples by DSC; PCL-DA imparts a lower Tm compared to 100% PLLA materials.

FIG. 18. Tensile modulus is plotted as a function of temperature at 37° C. and 80° C. PLLA materials have no significant difference in their tensile modulus; TS-MMS have a significantly lower tensile modulus at 80° C. attributed to the PCL-DA component.

FIG. 19A. Validation of TS-MMS properties in bulk (top) and maintenance of PLLA TIPS-induced nanofibers throughout deformation and recovery (bottom). FIG. 19B. The Young's modulus of the film sample is plotted as a function of cycling times.

FIG. 20. Working time of TS-MMS membranes increases significantly as a function of PCL-DA incorporation; 100% PLLA membranes have no TS-MMS properties.

FIG. 21. TS-MMS recovery as a function of PCL-DA molecular weight and thermal conditions.

FIG. 22. Thermosensitive cycle of TS-MMS scaffold testing process.

FIGS. 23A-23D. SEM micrographs illustrating internal structures of two TS-MMS scaffolds, shown in FIGS. 23A and 23B. FIGS. 23C and 23D show SEM micrographs illustrating the effect without heat treatment and with heat treatment on nanofibers, respectively.

FIG. 24. TS-MMS scaffold compressive moduli are a function of temperature, attributed to the incorporation of PCL-DA.

FIGS. 25A-25D. FIGS. 25A-25B show proof of concept for macropore recovery in a uniaxial deformation of TS-MMS scaffolds compared to control PLLA scaffolds. FIGS. 25C-25D show macropore recovery in a uniaxial deformation of two TS-MMS scaffolds shown in FIGS. 23A and 23B, respectively.

FIG. 26. Quantified proof-of-concept for macropore recovery in a uniaxial deformation of TS-MMS scaffolds compared to control PLLA scaffolds.

FIGS. 27A-27D. In a proof of concept molar tooth extraction model (FIG. 27A), TS-MMS scaffolds retain their favorable internal spherical macropores (FIGS. 27B-27C) and TIPS-nanofibers following deformation (FIG. 27D).

FIGS. 28A-28E. The TS-MMS scaffolds are able to fit various irregularly shaped defects after thermal induction and deformation.

FIG. 29. Schematic representation of TS-MMS membrane fabrication protocol resulting in bilayer structure.

FIG. 30. Bilayer structure of TS-MMS membranes with smooth top portion and nanofibrous bottom portion, continuous with each other in a single construct.

FIG. 31A-31I. SEM morphology of air-facing and glass-facing layers of TS-MMS membranes resulting from the open-face fabrication protocol (FIGS. 31A-31B); and SEM morphology of top and bottom layers of TS-MMS membranes resulting from the closed sandwich fabrication protocol (FIGS. 31C-31D). FIGS. 31E-31G show the thickness of the bilayers of TS-MMS membranes resulting from the open-face fabrication protocol. FIGS. 31H-31I show the impact of the whole film thickness on the bilayer morphology.

FIG. 32. Confocal laser microscopy of TS-MMS membrane cross section fabricated from fluorescently labelled PLLA and PCL-DA indicates their distribution throughout a construct.

FIG. 33. Schematic illustration of uniaxial directional temperature gradient which enables directional nanofiber formation during TIPS.

FIGS. 34A-34E. Oriented nanofibers due to uniaxial temperature gradient application during TIPS; degree of insulation of the film mold sides (strength of the gradient) determines the degree of nanofiber orientation.

FIGS. 35A-35B. SEM micrographs illustrate the morphologic differences between oriented and random nanofiber orientation in TS-MMS materials.

FIGS. 36A-36B. TIPS from benzene solvent allows for the formation of oriented tubules.

FIGS. 37A-37B. Gross microscopic images of virgin and partially degraded TS-MMS membranes indicates a biphasic degradation where the nanofiber layer degrades at an. accelerated rate compared to the air inhibited layer.

FIGS. 38A-38B. Scanning electron micrographs illustrate the rapid degradation of nanofibers in partially degraded (bottom) compared to virgin (top) TS-MMS membranes; the air-inhibited layer remains relatively intact during the same time frame.

FIG. 39. Thermogravimetric analysis (TGA) of PLLA degradation.

FIG. 40. Thermogravimetric analysis of TS-MMS (40% PCL-DA) degradation indicates a biphasic degradation profile.

FIG. 41. Plot of dW/dT from TGA of PLLA and TS-MMS membranes indicates a biphasic degradation profile of TS-MMS membrane compared to monophasic degradation of PLLA.

FIG. 42. TGA of PLLA scaffold degradation.

FIG. 43. TGA of TS-MMS (40% PCL-DA) degradation indicating a biphasic degradation profile.

FIG. 44. Plot of dW/dT from TGA of PLLA and TS-MMS scaffolds indicates a biphasic degradation profile of TS-MMS scaffold compared to monophasic degradation of PLLA.

FIGS. 45A-45B. FIG. 45A shows a photograph of mechanical testing apparatus used to assess suture retention strength from TS-MMS membranes. FIG. 45B shows the suture pull out tensile modulus for the TS-MMS (PLGA/PCL/PLLA) test film and the PLLA control film.

FIG. 46. Viability of bone marrow stromal cells (BMSCs) seeded to TS-MMS and PLLA membranes respectively, assessed by alamar blue proliferation assay.

FIG. 47. Viability of periodontal ligament mesenchymal stromal cells (PDL-SCs) seeded to TS-MMS and PLLA membranes respectively, assessed by alamar blue proliferation assay.

FIGS. 48A-48C. Scanning electron micrographs of PDL-SCs and BMSCs seeded to PLLA and TS-MMS nanofibers, respectively (top). The air inhibited layer (smooth, bottom) of TS-MMS membranes does not facilitate cell adhesion and proliferation.

FIGS. 49A-49B. Histologic sections of TS-MMS membranes seeded with PDL-SCs and BMSCs, respectively. Cells (red) are visualized in the nanofibrous layer but not air inhibited layer by confocal laser microscopy and phalloidin cytoskeletal fluorescent stain.

FIG. 50. SEM micrograph of nanoparticles fabricated from PLGA by a double emulsion method.

FIG. 51. Schematic representation of fabrication of TS-MMS scaffolds incorporating a particle drug delivery system as a part of the sugar sphere porogen method.

FIGS. 52A-52C. Bright field microscope (left), scanning electron microscopy (middle) and confocal laser microscopy (right) images of sugar sphere scaffold template incorporating PLGA nanoparticles loaded with Rhodamine B (red fluorescent, model drug) indicating its uniform incorporation.

FIGS. 53A-53B. Bright field (right) and fluorescence microscopy (left) images of TS-MMS scaffold fabricated with RhodamineB-loaded PLGA nanoparticles (red).

FIG. 54. Three dimensional reconstruction of confocal laser microscopy images taken in a Z-stack of TS-MMS scaffold loaded with RhodamineB PLGA nanoparticles demonstrating their uniform incorporation into the macropore walls.

FIGS. 55A-55C. SEM micrographs illustrating nanoparticle-in-nanofiber (NP-NF) morphology of PLGA nanoparticles embedded into TS-MMS scaffold nanofibers.

FIGS. 56A and 56B. Controlled release of Rhodamine B from TS-MMS scaffolds (n=17) incubated in PBS at 37° C. as shown in FIG. 56A. FIG. 56B shows the model drug (Rhodamine B) release kinetics from scaffold samples (n=17) with a nonlinear regression curve fit for logistic growth.

FIG. 57. Schematic illustration of controlled release from NP-NF drug delivery system.

FIGS. 58A-58B. Confocal laser microscopy in three dimensions (FIG. 58A) and two dimensions (FIG. 58B) illustrating the incorporation of FITC-labelled (fluorescent) bovine serum albumin (BSA protein) in PLGA nanoparticles, as a proof of concept for encapsulation of protein therapeutics.

FIGS. 59A-59F. Bright field (FIGS. 59A and 59D), fluorescence (FIGS. 59B and 59E) and cross sectional confocal laser microscopy (FIGS. 59C and 59F) images illustrating the advantageous uniformity of incorporation of PLGA-NPs into TS-MMS scaffolds by the NP-NF method compared to previously reported solvent wetting method which only allows for surface adhesion.

FIG. 60. Fluorescent microscope image of sugar sphere template incorporating two spatially distinct regions of FITC-loaded (green, left) and Rhodamine B-loaded (red, right) PLGA-NPs into the sugar sphere template as proof of concept for a bilayer drug delivery scaffold system.

FIG. 61. Confocal laser microscopy three dimensional reconstruction of the TS-MMS scaffold fabricated from the sugar sphere template visualized in FIG. 60.

FIG. 62. Schematic representation of NP-NF fabrication protocol for TS-MMS membranes whereby particle DDS is first applied in a thin film to a glass substrate, dried, then the TS-MMS is cast and synthesized in situ, incorporating NPs into its NF layer.

FIG. 63. Gross images of RhodamineB-loaded PLGA NPs applied in a thin film, from ethanol solvent, to a glass substrate prior to TS-MMS casting and synthesis.

FIG. 64. SEM micrograph of NP-NF morphology in TS-MMS membranes following the fabrication protocol described in FIGS. 62 and 63.

FIGS. 65A-65B. Confocal laser microscopy images of the nanofibrous layer of TS-MMS membranes incorporating FITC-BSA (protein, green, FIG. 65A) and Rhodamine B (small molecule, red, FIG. 65B) loaded PLGA NPs.

FIGS. 66A-66B. P(HEMA) nanoparticles are fabricated by in situ polymerization and observed by SEM (FIG. 66A); P(HEMA) nanoparticles incorporating 5% FITC-o-acrylate (green fluorophore) are incorporated into TS-MMS nanofibers by the same method described for PLGA NPs and the nanofibrous layer is visualized by confocal laser microscopy (FIG. 66B).

FIGS. 67A-67B. Versatility of TS-MMS membrane fabrication incorporating a particle DDS is illustrated with PLGA NPs (FIG. 67A, cross section, PLGA NP loaded with Rhodamine B) and P(HEMA) NPs (FIG. 67B, cross section, P(HEMA) NPs synthesized with 5% FITC-o-acrylate).

FIG. 68. TS-MMS membranes containing Rhodamine B-loaded PLGA NPs were dissected to separate the air inhibited layer from the nanofibrous layer and separately subjected to an accelerated degradation, then fluorescence intensity was measured. The nanofibrous layer contains the significant majority of PLGA NPs compared to the smooth layer.

FIGS. 69A-69B. The SEM images of the top face and bottom face of the TS-MMS film are shown in FIG. 69A. The SEM images of the cross-section of the top layer and the bottom layer are shown in FIG. 69B.

FIGS. 70A-70C. Introduction of PLGA causes a decrease in water contact angle with the TS-MMS film, reflecting increased wettability, as measured by a goniometer and quantified by image processing, as shown in FIGS. 70A-70C.

FIGS. 71A-71B. The stress-strain curves and tensile strength of different TS-MMS films having PLGA are shown in FIGS. 71A and 71B respectively

FIG. 72. The shape memory recovery time of different TS-MMS films having different amounts of PLGA.

FIGS. 73A-73B. The FITC BSA release kinetics from TS-MMS membranes having PLGA-DA/PCL-DA/PLLA (20/20/60) with water particle dispersion and ethanol particle dispersion are shown in FIGS. 73A and 73B, respectively.

FIGS. 74A-74D. FIG. 74A shows a SEM micrograph of nanoparticles fabricated from PLGA by a double emulsion method. FIG. 74B shows the experimental set up for testing the directional release of drug from the bilayer TS-MMS membrane. FIG. 74C shows the directional release of drug from the bilayer TS-MMS membrane. FIG. 74D shows the release kinetics comparison between two different PLGA/PCL/PLLA films having 20/20/60 and 0/40/60 weight ratios, respectively.

FIGS. 75A-75B. The SEM micrographs of TS-MMS membranes after 84 days of degradation (PBS, 37° C., shaker) for both the smooth top side and nanofibrous bottom side are shown in FIGS. 75A and 75B, respectively.

FIGS. 76A-76B. The impact of the PLGA on the crystallinity of the TS-MMS membranes having PLGA are shown in FIGS. 76A and 76B.

FIG. 77. The experimental process for in-vivo subcutaneous implantation.

FIG. 78. The controlled release of simvastatin from TS-MMS nanofibers.

FIG. 79. SEM images of the control and test samples are used to show the pore recovery after deformation, as shown in FIG. 79.

FIG. 80. The images of histologic sections (n>100 sections per sample) were taken with a stereomicroscope and analyzed by measuring pore circularity as shown in FIG. 80.

FIG. 81. Impact of pore recovery on cell and tissue infiltration after four weeks of time for different membranes.

FIG. 82. Light microscope images of the cell and tissue infiltration after four weeks of time after H&E staining for different membranes.

FIG. 83. PSR images of the cell and tissue infiltration after four weeks of time for different membranes.

FIGS. 84A-84I. FIG. 84A shows an image in that a full thickness mucoperiosteal flap was elevated from a midcrestal incision to uncover alveolar bone adjacent to the maxillary first molars, under a microscope. FIGS. 84B and 84C show the methods to create bone defects.

FIGS. 84D and 84E show the healthy control rat with incision but no defects and the rats with defects, respectively. FIG. 84F shows a membrane implantation on the defect. FIGS. 84G and 84H show 2D radiograph (reconstructed from uCT) of the defects at the mesial root in FIG. 84E for the healthy control rat and the rat with the defects after 3 weeks, respectively. FIG. 84 I shows a 3D reconstruction of the same defect.

FIGS. 85A-85D. 3D reconstruction of the defect shown in FIG. 84E after 4 weeks of treatment on the healthy rats (Group 1), rats with defects but without treatment (Group 2), and rats with bone defects treated with TS-MMS (Group 3) and TS-MMS having PDGF (group 4), respectively.

FIGS. 86A-86E. 3D reconstruction of the defect shown in FIG. 84E after 8 weeks of treatment on the healthy rats (Group 1), rats with defects but no treatment (Group 2), and rats with bone defects treated with TS-MMS (Group 3), TS-MMS having PDGF (group 4), and GuideOR control (Group 5), respectively.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

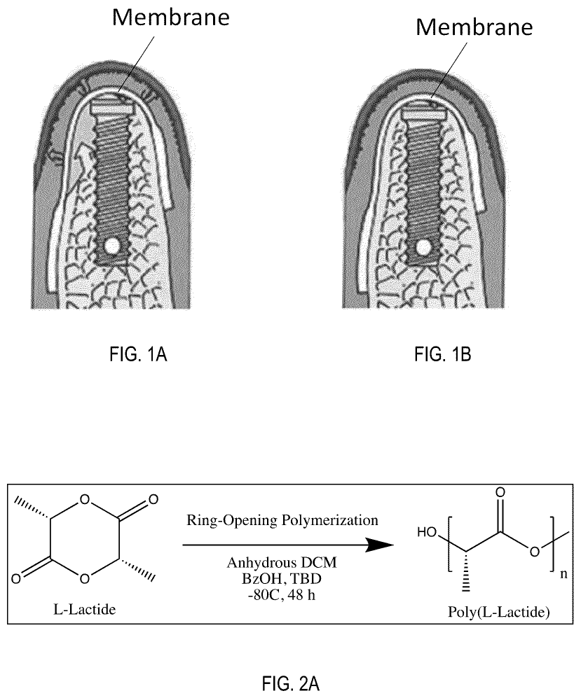

The present disclosure describes a novel tissue engineering matrix with improved clinical handling properties, designed for periodontal and craniofacial regeneration applications. In one application, these matrices are fabricated as biodegradable surgical membranes which serve not only as a protective barrier but also to induce regeneration through controlled release of inductive substances, facilitate regeneration through their tissue integration, and define and maintain dimensional stability in horizontal and/or vertical defects. In another application, these matrices are fabricated as biodegradable macroporous scaffolds, and the novel chemistry serves to deliver a three-dimensional environment capable of facilitating tissue ingrowth, vascularization, extracellular matrix deposition and remodeling, and tissue regeneration. An example of a surgical membrane (white) is shown in FIGS. 1A and 1B. The surgical membrane (white) is used as a partition in periodontal regeneration to allow selective healing of one compartment of tissue (bone, lower) while excluding epithelial cell infiltration (gingiva, upper). The osseous defects are shown in FIG. 1A. This concept of selective repopulation allows for complete regeneration (as shown in FIG. 1B) of osseous defects.

Clinical considerations for membrane selection include space creation and dimensional maintenance, defect isolation, and defect stabilization. While collagen membranes are biocompatible, they lack the ability to maintain space and dimensional stability. Furthermore, the fast degradation of collagen does not match the healing time of periodontal regeneration. The ideal regenerative matrix will: facilitate tissue integration to allow cellular and tissue ingrowth, and neotissue vascularization; isolate desired cells (epithelial exclusion) where indicated while not disrupting epithelial tissue integrity and vascularization; demonstrate clinically manageable handling properties which allow for predictable performance in the surgical environment; demonstrate space maintaining properties which provide a protected and defined volume for guided regeneration; and be biocompatible.

The thermosensitive matrix of the present disclosure described herein is novel and represents significant advancements compared to clinically available biomaterials. Advantageously, the thermosensitive matrix combines aspects of material conformability with rigidity and toughness and further has excellent clinical handling properties.

One aspect of the present disclosure provides a method of making a biodegradable polymeric material. The method comprises: reacting a polymer mixture comprising a first polymer and a second polymer, the first polymer being a biodegradable linear polymer and having first polymer chains, the second polymer being a biodegradable polymer having second polymer chains each of which has two or more functional groups, the first and second polymers being present in a weight ratio of between about 50:50 and about 99:1; and a reagent to form coupled second polymer chains in presence of the first polymer chains, thereby forming the biodegradable polymeric material, wherein the reagent is selected from a group of a radical initiator, a catalyst, a crosslinking agent, or a combination thereof, and wherein the biodegradable polymeric material has an interpenetrating polymer network (IPN) or semi-interpenetrating polymer network (SIPN) between the first and coupled second polymer chains, wherein the coupled second polymer chains are interspersed throughout the first polymer chains.

In embodiments, the second polymer is different from the first polymer. In embodiments, the second polymer may be a linear polymer and may have two terminus each of which may have a functional group. In embodiments, the second polymer is a branched polymer and has three or more terminus each of which may have a functional group at the terminus. In embodiments, the second polymer is a branched polymer and has as many terminus as there are branches, and each branch can have a functional group.

In embodiments, the reacting may include coupling the second polymer chains with each other through the two or more functional groups of the second polymer. In embodiments, the second polymer has two functional groups, and the reacting includes extending the second polymer chains. In embodiments, the second polymer has three or more functional groups, and the reacting includes extending and at least partially crosslinking the second polymer chains. As used herein, the term “coupling” includes, but is not limited to, extending the second polymer chains and/or at least partially crosslinking the second polymer chains.

In embodiments, the second polymer may have two functional groups, and the polymer mixture may further comprise a compound having two or more functional groups which can react with the two or more functional groups of the second polymer. The reacting comprises admixing the second polymer with the compound to thereby extend and/or partially crosslink at least a portion of the second polymer chains by coupling one or more second polymer chains with the compound and form the coupled second polymer chains including extended and/or partially crosslinked the second polymer chains. The biodegradable polymeric material can comprise an IPN or SIPN of the first polymer chains and the coupled second polymer chains; and the coupled second polymer chains are interspersed throughout the first polymer chains. The compound can be any small molecule chemical agent having two or more functional groups that can react with the two or more functional groups of the second polymer, such that two or more second polymer chains can react with the compound to thus extend and/or partially crosslink the second polymer chains through the compound.

In embodiments, the second polymer has two functional groups, and the polymer mixture may further comprise a third polymer having the third polymer chains each of which comprises two or more functional groups. In embodiment, the third polymer may have three or more functional groups which are reactive or non-reactive with the functional groups of the second polymer. The reacting may include admixing the second polymer and the third polymer to thereby (i) couple (a) at least a portion of the second polymer chains to each other; (b) at least a portion of the second polymer chains to at least a portion of the third polymer chains; and/or (c) at least a portion of the third polymer chains to each other, to thereby chain extend at least a portion of the second polymer chains and/or third polymer chains; and/or (ii) partially crosslink (a) at least a portion of the second polymer chains to each other; (b) at least a portion of the second polymer chains to at least a portion of the third polymer chains; and/or (c) at least a portion of the third polymer chains to each other, thereby forming the coupled second and third polymer chains. The biodegradable polymeric material may comprise an IPN of the first, second and third polymer chains. The coupled second and third polymer chains may be interspersed throughout the first polymer chains.

In embodiments, the functional groups of the second and third polymers and the compound may independently be any functional groups that have carbon-carbon double bonds which can be polymerized through radical polymerization (such as UV photo synthesis) in the presence of a radical initiator. Non-limiting examples of these functional groups having carbon-carbon double bonds include vinyl groups and acrylic groups, such as acrylate, methacrylate, or a combination thereof. For the second and third polymers, these functional groups of the second and third polymers may be linked to the polymer chains through an ester link or any other suitable link. The compound can have the same functional groups as those of the second and third polymers. Non-limiting examples of the compound may include: a difunctional acrylate such as hexandiol diacrylate, a trifunctional acrylate such as trimethylolpropane triacrylate (TMPTA), a tetrafunctional acrylate such as pentaerythritol tetraacrylate (PETA), a polyethylene glycol (PEG) acrylate with 2 or more arms such as 2-10 arms, or 2-6 arms, or a combination thereof. The compound can be used to extend and/or crosslink at least a portion of the second polymer chains.

When the functional groups of the second and third polymers and the compound are functional groups having carbon-carbon double bonds such as acrylate or methacrylate, the reagent is a radical initiator which can facilitate the free radical polymerization of the second polymer chains with each other, the third polymer chains with each other, the second polymer chains with the third polymer chains, or the second polymer chains with the compound by photo synthesis or thermal synthesis.

In embodiments, the functional groups of the second polymer may independently be any functional group that can react with a functional group of a third polymer or of a compound as disclosed herein, for example, through condensation, transesterification, click chemistry, or the like. Suitable examples of functional groups of the second polymer may include, but are not limited to, a hydroxyl (—OH), a carboxylic acid (—COOH), an epoxide, a thiol, an amine, an azide, an alkyne, a maleimide, a sulfhydryl, a vinyl sulfone, an acrylate, a phenol, a hydrazine, an aldehyde, or a combination thereof. The functional groups of the third polymer or the compound can be any functional groups that can react with the functional groups of the second polymer. The functional groups of the third polymer or compound may be, for example, a hydroxyl (—OH), a carboxylic acid (—COOH), an epoxide, a thiol, an amine, an azide, an alkyne, a maleimide, a sulfhydryl, a vinyl sulfone, an acrylate, a phenol, a hydrazine, an aldehyde, or a combination thereof. The functional groups of the second polymer and the functional groups of the third polymer or the compound may be a reactive pair, such as an alkene and thiol pair; a hydrazine and aldehyde reactive pair; an azide and alkyne reactive pair; an epoxide and thiol reactive pair; and an alcohol and carboxylic acid reactive pair. For example, the second polymer can be poly(caprolactone) (PCL) diol having di-hydroxyl functional groups, and the polymer mixture comprises a compound or third polymer which can be any compounds or polymer having two or more carboxylic acid functional groups. For example, the compound can be diacids (such as malonic acid, succinic acid, glutaric acid, 1,4-cyclohexanedicarboxylic acid, azelaic acid, or the like), and triacids (such as citric acid, isocitric acid, aconitic acid, or the like). When the functional groups of the second polymer chains and the functional groups of the third polymer chains or the compound are a reactive pair, the reagent may be a catalyst to facilitate the reactions between the reactive pair, such as the condensation, transesterification, or click chemistry reactions, or the like.

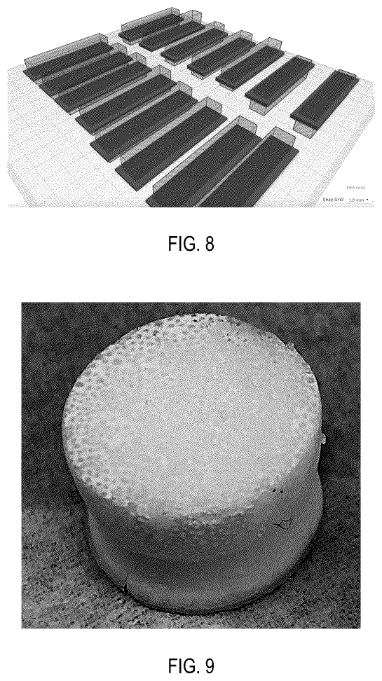

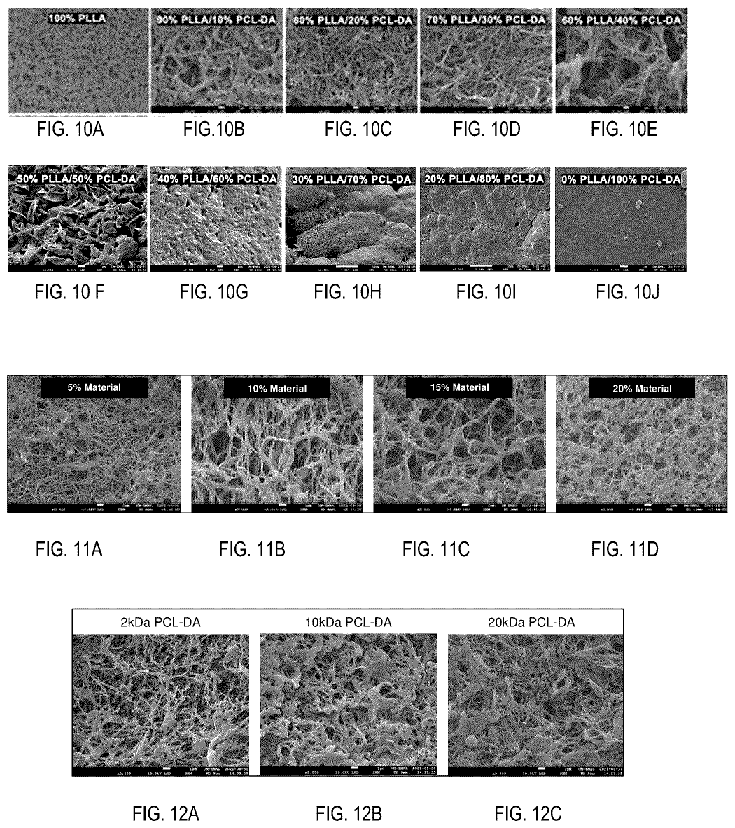

In embodiments, the first polymer is at least partially crystallized in the biodegradable polymeric material and has a crystallinity of about 10-100%, or about 20-90%, or about 20-80%, or about 30-70%, or about 40-60%. In embodiments, the first polymer is at least partially phase separated from the coupled second and/or third polymers and forms nanostructures (including nanofibers and nanotubes) or microtubes dispersed in the biodegradable polymeric material. As used herein, the term “phase separated” refers to the formation of crystalline nanostructures of the first polymer which is separated from the amorphous second and/or third polymers in nanoscale level, for example as shown in FIGS. 10B-10E.

In embodiments, the second polymer may be a linear biodegradable polymer having second polymer chains each of which has two terminuses and two terminal functional groups each of which is at one of the two terminuses, and the first and second polymers may be present in the weight ratio in a range of between about 50:50 and about 99:1, or between about 60:40 and about 90:10, or between about 60:40 and about 80:20.

As used herein, the term “between” in the context of a range is inclusive of the two ends of the range, unless specified otherwise.

In embodiments, the method may comprise: 1) admixing a first polymer with a second polymer in a weight ratio of between about 50:50 and about 99:1 to form a polymer mixture; and 2) reacting the polymer mixture with the reagent to form the biodegradable polymeric material. In embodiments, the admixing may include admixing the first and second polymers by melting mixing or by dissolving and mixing in a first organic solvents to form a polymer mixture solution.

In embodiments, the method may comprise: 1) dissolving and mixing the first and second polymers in a first organic solvents to form a polymer mixture solution; 2) dissolving the reagent in a second organic solvent to form a reagent solution; 3) admixing the polymer mixture solution and the reagent solution to form a polymer-reagent solution; and 4) reacting the polymer-reagent solution to form coupled second polymer chains, thereby forming the biodegradable polymeric material.

In embodiments, the reagent may be a radical initiator, a catalyst, a crosslinking agent, or a combination thereof. In embodiments, the reagent is a catalyst which can catalyze the polymerization/coupling of the second polymer; or the reaction between the functional groups of the second polymer and the functional groups of the third polymer or the compound. In embodiments, the reagent may be provided in an amount of about 0.01-20 wt. %, or about 0.1-10 wt. %, or about 0.1-5 wt. %, or about 1 wt. %, based on the total dry weight of the polymer-reagent mixture. In embodiments, the reagent is a crosslinking agent have two or more functional groups which are reactive with the functional groups of the second polymer and can be the same functional groups as those of the third polymer or compound described herein above.

In embodiments, the reagent may be a radical initiator, and the reacting may comprise admixing the radical initiator with the polymer mixture to form a polymer-initiator mixture. In embodiments, the radical initiator is provided in an amount of about 0.01-10 wt. %, or about 0.1-5 wt. %, or about 1 wt. %, based on the total weight of the polymer-initiator mixture.

In embodiments, the radical initiator is a photo initiator. In embodiments, the photo initiator is selected from the group of 2-hydroxy-4′-(2-hydroxyethoxy)-2-methylpropiophenone (such as Irgacure 2959) having an excitation wavelength of 365 nm, 1-vinyl-2 pyrrolidinone (NVP) having an excitation wavelength of 365 nm, Lithium phenyl-2,4,6-trimethylbenzoylphosphinate (LAP) having an excitation wavelength of 405 nm, and a combination thereof.

In embodiments, the reacting may include irradiating the polymer-initiator mixture with UV lights to form the biodegradable polymeric material. In embodiments, the UV light has a wavelength in a range of about 10-420 nm, about 100-420 nm, about 250-420 nm, about 100-400 nm, about 250-400 nm, or about 250 nm-365 nm. In embodiments, the UV light has an intensity of between about 25 microjoules (μJ) and about 10M μJ.

In embodiments, the reacting comprises melting and blending the polymer-initiator mixture at a temperature in a range of about 50° C. to about 150° C., about 80° C. to about 120° C., or about 100° C. and about 120° C.

In embodiments, the reacting further comprises admixing stannous octanoate (Sn(Oct)₂) with the polymer-initiator mixture, and Sn(Oct)₂ and the radical initiator are provided in a ratio in a range of between about 0.01:100 and 20:100 mol/mol, or about 0.1:100 and 10:100, or about 0.1:100 and 5:100, or about 1:100 mol/mol.

In embodiments, the first polymer has a first molecular weight between 500 Da and 200 KDa before and after the reacting. In embodiments, the molecular weight of the first polymer after the reacting is substantially the same (or unchanged) as the molecular weight of the first polymer before the reacting.

In embodiments, the second polymer has an initial molecular weight between 500 Da and 200 KDa before the reacting and has a final molecular weight between 20 KDa and 500 KDa after the reacting, and the final molecular weight is greater than the initial molecular weight of the second polymer. In embodiments, the final molecular weight of the second polymer is about 1.0-100, or about 1.0-50, or about 1.0-10 times greater than the initial molecular weight of the second polymer. In embodiments, the second polymer is in situ-polymerized or coupled through the two or more functional groups (in some embodiments, the two or more terminal functional groups) after the reacting, and the final molecular weight is about 1.5-10, or about 2.0-10 times greater than the initial molecular weight.

In embodiments, the in situ-polymerizing or coupling includes but is not limited to the chain-extending and/or at least partially crosslinking the second polymer chains. As used herein and unless specified otherwise, the terms “chain-extension” or “extending” or “chain-extending” refer to the lengthening of a second polymer chain by coupling one or more additional second polymer chains to the second polymer chain through the two or more functional groups (or the terminal functional groups) to provide a second polymer chain lengthened by the one or more additional second polymer chains, having a higher molecular weight than that of the initial second polymer chain. In embodiments, in-situ polymerization may include at least partially crosslinking the second polymer chains.

In embodiments, after the reacting, the second polymer chains are at least partially crosslinked with each other within a matrix of the first polymer, and the second polymer has a crosslinking density of about 0.1-50%, or about 1-20%, or about 1-15%, or about 5-10%. In embodiments, the crosslinking of the second polymer chains with each other is through the two or more functional groups of the second polymer.

In embodiments, the reagent is the crosslinking agent having two or more reactive groups capable of reacting with the two or more functional groups of the second polymer. In embodiments, the chain-extending of the second polymer chains is through the reaction between the two or more functional groups of the second polymer and the two or more reactive groups of the crosslinking agent. In embodiments, the crosslinking of the second polymer chains is through the reaction between the two or more functional groups of the second polymer and the two more reactive groups of the crosslinking agent.

As used herein, and unless specified otherwise the term “partially” refers to a range of more than 0% and lower than 100%.

In embodiments, the method further comprises cooling the biodegradable polymeric material to form crystalline nanofibers of the first polymer dispersed in the IPN or SIPN of the biodegradable polymeric material. After cooling, the first polymer is at least partially phase separated from the second polymer, and has a crystallinity of about 10-90%, or about 20-80%, or about 40-50%. In embodiments, the first polymer may form crystalline nanostructures. In embodiments, the first polymer may form crystalline nanofibers which are about 10-90 wt. %, or about 10-80%, or about 20-60 wt. % of the first polymer. In embodiments, before the reacting, the first polymer has a crystallinity of about 40-100%, about 50-100%, or about 80-95% at room temperature or about 25° C.

As used herein, the term “room temperature” may refer to a temperature in a range of 25° C.±5° C., or 25° C.±3° C.

In embodiments, the cooling comprises applying a uniaxial temperature gradient to the biodegradable polymeric material.

In embodiments, the crystalline nanofibers are oriented substantially along the direction of the uniaxial temperature gradient.

In embodiments, the crystalline nanofibers are oriented along or at an angle of about 250 or less relative to the direction of the uniaxial temperature gradient.

In embodiments, the biodegradable polymeric material is fabricated as a membrane in a plane, the uniaxial temperature gradient is in a vertical direction perpendicular to the plane, and the crystalline nanofibers are oriented substantially along the vertical direction or at an angle about 250 or less relative to the vertical direction.

As used herein, the term “oriented substantially” along a direction refers to an orientation along, or parallel to, the direction or within an angle of about 250 or less relative to the direction.

In embodiments, the first polymer has a melting point between about 60° C. and about 350° C., or between about 70° C. and about 300° C., or between about 70° C. and about 220° C., or between about 140° C. and about 180° C. before and after the reacting. The second polymer has a melting point between about 40° C. and about 100° C., or between about 42° C. and about 75° C., or between about 48° C. and 55° C., or between about 50° C. and 55° C. before and after the reacting.

In embodiments, the melting point of the first polymer remains substantially unchanged by the reacting.

In embodiments, the melting point of the second polymer remains substantially unchanged by the reacting.

As used herein, the term “substantially unchanged” by a process (e.g., reacting or heating) refers to a change in value of a characteristic of less than 20%, for example, less than 10%, less than 5%, less than 1%, less than 0.5%, or less than 0.1% relative to the value of the characteristic before the process.

In embodiments, the second polymer is characterized by a first melting point before the reacting and a second melting point after the reacting, and the second melting point is higher than the first melting point. In embodiments, the second melting point of the second polymer is about 0-20° C., or about 0-10° C., or about 0.1-5° C. higher than the first melting point of the second polymer.

In embodiments, the melting point of the second polymer after the reacting is increased by about 0-20° C., about 0-10° C., about 0-5° C., or about 0.1-3° C. as compared to the melting point of the second polymer before the reacting.

In embodiments, the first polymer before the reacting has an initial first tensile modulus at about 25° C. and has a final first tensile modulus at a temperature about 1-5° C. higher than the melting point of the second polymer before the reacting, and the final first tensile modulus is substantially the same as, or about 90-100% of, or about 95-105% of, or about 95-100% of, the initial first tensile modulus.

In embodiments, the first polymer is characterized by a tensile modulus that remains substantially unchanged when heated from about 25° C. to a temperature up to the melting point of the second polymer.

In embodiments, the first polymer is characterized by a tensile modulus that remains substantially unchanged when heated from about 25° C. to a temperature about 0-30° C., about 0-25° C., about 0-20° C., about 0-15° C., about 0-10° C., or about 0-5° C. higher than the melting point of the second polymer.

In embodiments, the first polymer is characterized by a change in tensile modulus of less than about 10% or about 5% when heated to a temperature about 1-10° C., or about 1-5° C. higher than the melting point of the second polymer, relative to the tensile modulus of the first polymer at about 25° C.

In embodiments, before the reacting the second polymer has an initial second tensile modulus at about 25° C. and has a final second tensile modulus at a temperature about 1-5° C. higher than the melting point of the second polymer before the reacting, and the final second tensile modulus decreases by at least about 10%, or by 20-80%, or by about 30-40%, compared to the initial second tensile modulus.

In embodiments, before the reacting, the second polymer has an initial second tensile modulus at about 25° C. and a final second tensile modulus at a temperature about 1-5° C. higher than the melting point of the second polymer, and the final second tensile modulus is at least about 10%, or by 20-80%, or about 30-40%, lower than the initial second tensile modulus.

In embodiments, the second polymer after the reacting has an initial third tensile modulus at about 25° C. and has a final third tensile modulus at the elevated temperature, and the final third tensile modulus decreases by at least about 10%, or by 20-80%, or by about 30-40%, compared to the initial third tensile modulus.

In embodiments, after the reacting, the second polymer has an initial third tensile modulus at about 25° C. and a final third tensile modulus at a temperature about 1-5° C. higher than the melting point of the second polymer before the reacting, wherein the final third tensile modulus is at least about 20%, or by about 30-40%, lower than the initial third tensile modulus.

In embodiments, the biodegradable polymeric material is characterized by a first tensile strength which remains substantially unchanged in a temperature range of about 25° C. to a temperature about 1-5° C. lower than the melting point of the second polymer before the reacting.

In embodiments, the biodegradable polymeric material is characterized by a second tensile modulus at a temperature about 1-5° C. higher than the melting point of the second polymer before the reacting, wherein the second tensile strength is at least about 20%, or about 30-40%, lower than the first tensile modulus.

In embodiments, the biodegradable polymeric material is in a first state and has a high tensile strength which is substantially unchanged in a temperature range of about 25° C. to an intermediate temperature.

In embodiments, the biodegradable polymeric material is in a second state and has a low tensile strength which is at least about 20%, or about 30-40%, lower than the high tensile modulus at the elevated temperature. The biodegradable polymeric material has a unique thermoresponsive mechanical property/behavior.

In embodiments, the elevated temperature is about 1-10° C., or about 1-5° C. higher than the melting temperature of the second polymer before the reacting, and the intermediate temperature is a temperature about 1-10° C., or about 1-5° C. lower than the melting temperature of the second polymer before the reacting.

In embodiments, the biodegradable polymeric material has an original shape as fabricated and the biodegradable polymeric material maintains at least about 90%, about 95%, or about 95-100% of the original shape after at least about 10, about 25, about 50, or about 100 repeated cycles of heating, deforming and cooling the biodegradable polymeric material, wherein the heating includes heating to a temperature at or above the melting point of the second polymer before the reacting and below the melting point of the first polymer, wherein the deforming occurs at the temperature above the melting point of the second polymer before the reacting and below the melting point of the first polymer, and wherein the cooling includes cooling to about 25° C.

In embodiments, the biodegradable polymeric material has an original shape as fabricated, the biodegradable polymeric material is capable of retaining to at least about 90%, or at least about 95%, or in a range of about 90-100% of the original shape after at least about 10, about 25, about 50 or about 100 repeated cycles of the heating, deforming, and cooling the biodegradable polymeric material. In embodiments, the biodegradable polymeric material has a shape memory property or a memorized microstructure (MMS). As used herein, and unless specified otherwise, a material has a “shape memory property” or a “memorized microstructure” if the material reverts back to, and thereby maintains, at least 90% of its original structure after at least about 10 cycles of heating the material to a temperature in a range of 40° C. to 75° C., deforming at the elevated temperature, and cooling the material to room temperature.

In embodiments, the heating includes heating to a temperature at or above the melting temperature of the second polymer before the reacting and below the melting temperature of the first polymer, such as heating to a temperature between about 42° C. and about 75° C., or between about 45° C. and about 70° C., between about 45° C. and about 70° C., or between about 45° C. and about 70° C.

In embodiments, the first polymer can be any biodegradable and biocompatible polymer which has a melting point in a range of about 70-250° C., or about 75-220° C., or about 80-200° C., or about 140-180° C., and has a bulk crystallinity of at least 20%, or about 20-100%, or about 20-80% at about room temperature. The first polymer is further capable of forming nanofibrous structures in sufficient conditions.

In embodiments, the first polymer may comprise poly (L-lactic acid) (PLLA), poly-3-hydroxybutyrate (P3HB), poly(lactic-co-glycolic acid) (PLGA), a copolymer containing PLLA having a bulk crystallinity of at least about 20%, or about 20-100%, or about 20-80%, or a combination thereof. In embodiments, the copolymer is capable of forming crystalline nanofibers when cooling to room temperature in sufficient conditions.

In embodiments, the first polymer is poly (L-lactic acid) (PLLA). In embodiments, the PLLA is substantially free of poly (D-lactic acid) (PDLA). In embodiment, the PLLA has a purity of at least about 85 wt. %, at least about 90 wt. %, or at least about 95 wt. %, or at least about 98 wt. %, or at least 99 wt. %, or at least 99.5 wt. %. Without intending to be bound by theory, it is believed that PLLA having a purity higher than about 90 wt. % (especially higher than 99 wt. %) and is substantially free of PDLA, is more biocompatible with the human or animal bodies and would cause less negative immune cellular response than PLLA having a purity lower than 90%.

As used herein, the term a degree of “bulk crystallinity” refers to a degree of crystallinity in the bulk of the polymeric material. As used herein, the term “surface crystallinity” refers to a degree of crystallinity in the surface of the polymeric material.

In embodiments, the second polymer can be any biodegradable and biocompatible polymer which has a melting point in a range of about 40-90° C., or about 45-80° C., or about 50-75° C., or about 50-60° C. The melting point of the second polymer may be at least about 20° C., or about 25° C., or about 30° C., or about 35° C., or about 40° C., or about 50° C. lower than the melting point of the first polymer, or about 30-150° C., or about 30-120° C. lower than the melting point of the first polymer.

In embodiments, the second polymer may comprise poly-ε-caprolactone diacrylate (PCL-DA), poly-ε-caprolactone triacrylate (PCL-TA), poly(lactic-co-glycolic acid) diacrylate (PLGA-DA), poly(lactic-co-glycolic acid) triacrylate (PLGA-TA), comprise poly-4-hydroxybutyrate (P4HB), poly-4-hydroxybutyrate diacrylate (P4HB-DA), poly-4-hydroxybutyrate triacrylate (P4HB-TA), polyethylene glycol (PEG) acrylate with 2 or more arms, or a combination thereof.

In embodiments, the second polymer may comprise poly-ε-caprolactone diacrylate (PCL-DA), and the third polymer may comprise poly-ε-caprolactone triacrylate (PCL-TA). In embodiments, the poly-ε-caprolactone diacrylate (PCL-DA) in the biodegradable polymeric material is chain extended after the reacting. In embodiments, the poly-ε-caprolactone triacrylate (PCL-TA) in the biodegradable polymeric material after the reacting is chain extended and at least partially crosslinked and has a crosslinking density of about 1-20%, or about 5-15% or about 10%.

In embodiments, the polymer mixture may comprise poly-ε-caprolactone diacrylate (PCL-DA) and poly-ε-caprolactone triacrylate (PCL-TA) in a weight ratio of between 100:0.1 and 100:50, or between 100:1 and 100:20, or between 100:1 and 100:15, or between 100:1 and 100:10, of between 100:5 and 100:10. In embodiments, the poly-ε-caprolactone diacrylate (PCL-DA) and poly-ε-caprolactone triacrylate (PCL-TA) in the biodegradable polymeric material after the reacting are chain extended and at least partially crosslinked and has a crosslinking density of about 1-20%, or about 5-15% or about 10%.

In embodiments, the second polymer is poly-ε-caprolactone diacrylate (PCL-DA) prepared from a process comprising: providing poly-ε-caprolactone (PCL) diol having a terminal hydroxyl functional group at each terminus of substantially each polymer chain of the PCL; and modifying the poly-ε-caprolactone (PCL) diol to convert at least part of the terminal hydroxyl functional groups at its terminus to acrylic functional groups through nucleophilic substitution reaction with acryloyl chloride or methacrylic anhydride in presence of triethyl amine (TEA) at about 0° C. for about 12 hours to yield poly-ε-caprolactone diacrylate (PCL-DA).

As used herein, the term “substantially each” polymer chain may refer to at least 70%, or at least 80%, or at least 90%, or at least 95%, or 100% of the polymer chains.

In embodiments, the second polymer may be or comprise poly-4-hydroxybutyrate (P4HB). In embodiments, the second polymer may be or comprise poly-4-hydroxybutyrate diacrylate (P4HB-DA), or hydroxybutyrate triacrylate (P4HB-TA).

In embodiments, the first polymer may comprise poly (L-lactic acid) (PLLA), and the second polymer may comprise one of poly-ε-caprolactone diacrylate (PCL-DA) and poly-ε-caprolactone triacrylate (PCL-TA).

In embodiments, the polymer mixture may comprise the first, second and third polymers. The first polymer may comprise poly (L-lactic acid) (PLLA); the second polymer may comprise one of poly-ε-caprolactone diacrylate (PCL-DA) and poly-ε-caprolactone triacrylate (PCL-TA); and the third polymer may comprise PLGA-DA and/or PLGA-TA. The PLLA is present in an amount of about 50-99 wt. %, or about 55-95 wt. %, or about 55-90 wt. %, or about 60-90 wt. %, or about 60-80 wt. % in the polymer mixture, and/or the resulting biodegradable polymeric material.

The biodegradable polymeric material may be designed to adjust the degradation rate in physiologic environment for different applications. For example, the biodegradable polymeric material having IPN or SIPN of PLLA/PCL may be suitable for long term drug delivery applications. This is because both the PLLA and PCL polymers have long degradation time. PLLA has an approximate degradation time of over 24 months. The PCL polymer has a degradation time of approximately two to three years. The biodegradable polymeric material may require different degradation rates for different applications. The degradation rate of the biodegradable polymeric material can be altered by adding other suitable biodegradable polymers or copolymers to the PLLA/PCL system or replacing one or both of the PLLA and PCL polymers. One suitable biodegradable polymer is PLGA which has a tailorable degradation time from about a few weeks to several months by varying the poly(lactic acid) to poly(glycolic acid) ratio. The PLGA polymer can be used to replace part or all of the PCL polymer as detailed in Examples 3 and 4 below. The PLGA is added to the biodegradable polymeric material to increase the degradation rate of the biodegradable polymeric material.

Another aspect of the present disclosure provides a biodegradable polymeric material comprising: a first polymer and a second polymer provided in a weight ratio of between about 50:50 and about 99:1, or between about 60:40 and about 80:20, wherein the first polymer is a biodegradable linear polymer having first polymer chains and a crystallinity of about 10-100%, about 20-90%, or about 20-80%, or about 30-70%, or about 40-60%, wherein the second polymer is a biodegradable polymer different from the first polymer and having second polymer chains each of which comprises two or more functional groups, wherein at least a portion of the second polymer chains are coupled to each other through the functional groups, wherein the biodegradable polymeric material has an interpenetrating polymer network (IPN) or a semi-interpenetrating polymer network (SIPN) between the first and second polymer chains, and the second polymer chains are interspersed throughout the first polymer chains. In embodiments, the second polymer may be at least partially crosslinked with each other and may have a crosslinking density of about 0.1-50%, or about 1-20%. In embodiments, the biodegradable polymeric material may have a third polymer, and may have IPN or SIPN between the first, second and third polymer chains, and the third polymer chains may be at least partially crosslinked with itself and/or with the second polymer chains. The crosslinking density of the third polymer and/or the second polymer is in a range of about 0.1-50%, or about 1-20%. In embodiments, the biodegradable polymeric material may comprise a compound having two or more functional groups which can react with the functional groups of the second polymer to couple at least a portion of the second polymer chains by the compound. The coupling of the second polymer chains by the compound may include but is not limited to extending and/or partially crosslinking a portion of the second polymer chains by the compound.

The selection and properties of the first, second and/or third polymers, and/or the compound are described herein above.

In embodiments, the second polymer each may have two or more functional groups and are coupled (including chain-extended and/or at least partially crosslinked) with each other through the two or more functional groups. In embodiments, the second polymer chains may be at least partially crosslinked with each other and have a crosslinking density of about 0.1-50%, or about 1-20%. In embodiments, the biodegradable polymeric material may have the IPN or SIPN between the first polymer chains and the coupled (including chain-extended and/or crosslinked) second polymer chains, and the coupled second polymer chains are interspersed throughout the first polymer chains.

In embodiments, the second polymer is a linear polymer. In embodiments, the second polymer may each have two terminus and two terminal functional groups each of which is at one of the two terminus. The second polymer chains are coupled (chain extended) with each other through the terminal functional groups.

In embodiments, the second polymer is a branched polymer having three or more branches each of which has a terminus. Each terminus or substantially each terminus may have a terminal functional group. In embodiment, two or more of the terminus may each have a functional group.

In embodiments, the first polymer is at least partially crystallized and at least partially phase separated from the second polymer, the first polymer has crystalline nanofibers homogeneously dispersed in the interpenetrating polymer network (IPN) of the biodegradable polymeric material, and wherein the crystalline nanofibers are about 10-90 wt. %, or about 10-80 wt. %, or about 20-70% wt. %, or about 30-60 wt. % of the first polymer.

In embodiments, the first polymer may be at least partially phase separated from the second polymer, and about 10-80 wt. % of the first polymer may be in the form of crystalline nanostructures that are dispersed in the interpenetrating polymer network (IPN) of the biodegradable polymeric material. In embodiments, the crystalline nanostructures may be homogeneously dispersed in the IPN.

In embodiments, the crystalline nanostructures are crystalline microtubes and/or nanotubes.

In embodiments, the crystalline nanostructures are crystalline nanofibers. In embodiments, the crystalline nanofibers are oriented along a predetermined direction. In embodiments, the crystalline nanofibers are oriented parallel to a predetermined direction or within an angle of about 0-25° or about 0-15° from the predetermined direction. In embodiments, the crystalline nanofibers are oriented substantially parallel to each other or within an angle of about 0-30°, about 0-25°, about 0-20°, about 0-15°, or about 0-10° from an axis of the biodegradable polymeric material.

In embodiments, the biodegradable polymeric material is in a plane and the crystalline nanofibers are oriented along a direction substantially perpendicular to the plane, or along a direction at an angle of about 65-115° relative to the plane.

In embodiments, the first polymer has a melting point of about 60-350° C., or about 65-300° C., or about 70-200° C., about 100-190° C., or about 140-180° C. In embodiments, before the reacting, the second polymer has a melting point of about 40-90° C., or about 42-75° C., or about 45-60° C., or about 48-55° C., or about 50-55° C.

In embodiments, the first polymer is characterized by an initial first tensile modulus at about 25° C. and a final first tensile modulus at a temperature about 1-5° C. higher than the melting point of the second polymer, wherein the final first tensile modulus is about 95-105% of the initial first tensile modulus.

In embodiments, the second polymer is characterized by an initial second tensile modulus at about 25° C. and a final second tensile modulus at a temperature about 1-5° C. higher than the melting point of the second polymer, and the final second tensile modulus is at least about 20%, or about 30-40% lower than the initial second tensile modulus.

In embodiments, the biodegradable polymeric material is characterized by a first tensile modulus which remains substantially unchanged in a temperature range of about 25° C. to a temperature about 1-5° C. lower than the melting point of the second polymer.

In embodiments, the biodegradable polymeric material has thermoresponsive mechanical property.

In embodiments, the biodegradable polymeric material is characterized by a second tensile modulus at a temperature about 1-5° C. higher than the melting point of the second polymer, wherein the second tensile modulus is at least about 20% (30-40%) lower than the first tensile modulus.

In embodiments, the biodegradable polymeric material has an original shape as fabricated and the biodegradable polymeric material maintains at least about 90% of the original shape after at least about 10, about 20, about 30, about 40, about 50, or about 100 repeated cycles of heating, deforming, and cooling the biodegradable polymeric material, wherein the heating includes heating to a temperature above the melting point of the second polymer and below the melting point of the first polymer, wherein the deforming occurs at the temperature above the melting point of the second polymer and below the melting point of the first polymer, and wherein the cooling includes cooling to about 25° C.

In embodiments, the first polymer is poly (L-lactic acid) (PLLA) and the second polymer is poly-ε-caprolactone diacrylate (PCL-DA). In embodiments, the biodegradable polymeric material may have the IPN or SIPN between polymer chains of the PLLA and the coupled PCL-DA. In embodiments, the PLLA is at least partially phase separated from the coupled PCL-DA.

In embodiments, the first polymer may be poly (L-lactic acid) (PLLA) and the second polymer may be poly-ε-caprolactone triacrylate (PCL-TA), and the PCL-TA may be chained-extended and at least partially crosslinked and have a crosslinking density of about 1-20%. In embodiments, the biodegradable polymeric material may have the IPN or SIPN between polymer chains of the PLLA and the coupled (including chain-extended and/or at least partially crosslinked) PCL-TA. In embodiments, the PLLA may be at least partially phase separated from the coupled PCL-TA.

In embodiments, the PLLA in the biodegradable polymeric material may have a crystallinity of between about 20-80 wt. % and about 20-80 wt. %. In embodiments, the crystallized PLLA may be in the form of crystalline nanofibers homogeneously dispersed in the IPN or SIPN

Another aspect of the present disclosure provides a method of making a biodegradable surgical membrane, the method comprising: admixing a polymer mixture and a first organic solvent to form a homogeneous polymer mixture solution; admixing a reagent solution comprising a reagent and a second organic solvent with the polymer mixture solution to form a polymer-reagent solution; and reacting the polymer-reagent solution thereby forming the biodegradable surgical membrane, wherein the polymer mixture comprises a first polymer and a second polymer in a weight ratio in a range of about 50:50 and about 99:1, the first polymer being a biodegradable linear polymer having first polymer chains, the second polymer being a biodegradable polymer different from the first polymer and having second polymer chains each of which has two or more reactive functional groups, wherein the reacting includes coupling (including extending and/or at least partially crosslinking) the second polymer chains with each other through the two or more functional groups to form coupled second polymer chains, wherein the biodegradable surgical membrane has an interpenetrating polymer network (IPN) or semi-interpenetrating polymer network (SIPN) between the first polymer chains and the coupled (chain-extended and/or at least partially crosslinked) second polymer chains.

In come embodiments, the polymer mixture solution comprises about 1-30 wt. % of the polymer mixture and about 70-99 wt. % of the first organic solvent each by weight of the polymer mixture solution.

In embodiments, the reagent is a radical initiator, a catalyst, a crosslinking agent, or a combination thereof. In embodiments, the reagent is a radical initiator, and the polymer-reagent solution is a polymer-initiator solution. In embodiments, the radical initiator may be a photo initiator. In embodiments, the reacting includes irradiating the polymer-initiator solution with a UV light thereby forming the biodegradable surgical membrane. In embodiments, the reacting may include at least partially crosslinking the second polymer. In embodiments, the second polymer has two functional groups, the polymer mixture comprises a third polymer having third polymer chains each of which has three or more functional groups which may be reactive or non-reactive with the functional groups of the second polymer. The reacting may include (i) coupling (a) at least a portion of the second polymer chains to each other; (b) at least a portion of the second polymer chains to at least a portion of the third polymer chains; and/or (c) at least a portion of the third polymer chains to each other, to thereby chain extend at least a portion of the second polymer chains and/or third polymer chains; and/or (ii) partially crosslinking (a) at least a portion of the second polymer chains to each other; (b) at least a portion of the second polymer chains to at least a portion of the third polymer chains; and/or (c) at least a portion of the third polymer chains to each other. The biodegradable polymeric material comprises an IPN of the first, second and third polymer chains, and the second and third polymer chains are interspersed throughout the first polymer chains.

In embodiments, the method further comprises casting the polymer-reagent solution (or the polymer-initiator solution) into a film mold.

In embodiments, the polymer mixture solution and the reagent solution (or the radical initiator solution) may be mixable and miscible.

In embodiments, the reagent is a photo initiator, and the polymer-reagent solution is a polymer-initiator solution. The reacting may include irradiating the polymer-initiator solution, and the irradiating may facilitate coupling of the second polymer chains with each other through the two or more functional groups. In embodiments, the coupled second polymer in the biodegradable surgical membrane may have a crosslinking density of about 0.1-20%, or about 1-10%. As used herein, the term “coupling” includes not is not limited to chain-extending and/or partially crosslinking the second polymer chains.

In embodiments, the method may comprise heating the polymer-reagent solution (or polymer-initiator solution) to a temperature of about 40-90° C., or about 50-70° C., before the irradiating and/or the casting.

In embodiments, the method may comprise one or more of the steps of: cooling the biodegradable surgical membrane to a temperature lower than room temperature, warming the biodegradable surgical membrane to the room temperature; removing the biodegradable surgical membrane from the film mold; and drying or lyophilizing the biodegradable surgical membrane.

In embodiments, the first polymer in the biodegradable surgical membrane may be at least partially phase separated from the second polymer and may have about 20-80% crystalline. In embodiments, about 20-80% of the first polymer may be in the form of crystalline nanostructures or microtubes. The crystalline nanostructures may include nanofibers. The crystallized first polymer may be in a form of microtubes or nanotubes.

In embodiments, the film mold may have a bottom wall and four side walls, and does not have a cover and is thus open to the environment. The biodegradable surgical membrane may have a bi-layer morphology with an air-inhibited smooth layer having a porosity no more than 40% v/v and a porous layer having a porosity of at least 60% v/v when the film mold is open to the environment. In embodiments, the film mold may have a cover and is thus closed to the environment. The biodegradable surgical membrane may have a homogeneous porosity morphology without layers of different porosity when the film mold is closed to the environment.

In embodiments, the cooling comprises: cooling the film mold containing the biodegradable surgical membrane at a temperature between about 0° C. and about −120° C., or between about −40° C. and about −90° C., for about 1-60 hours, for example 48 hours.

In embodiments, the side walls of the film mold are insulated, and the cooling comprises: placing the bottom of the film mold containing the biodegradable surgical membrane on a cold surface having a temperature between about −40° C. and about −100° C., or between −60° C. and −90° C. for about 10 minutes to 10 hours; and immersing the film mold containing the biodegradable surgical membrane into a liquid having a temperature between about −30° C. and about −100° C., or between about −60° C. and about −90° C. for about 30-60 hours. In embodiments, the liquid is ethanol or methanol. In embodiments, the immersing may include partially immersing the film mold containing the biodegradable surgical membrane into the liquid, such that the side walls of the film mold are in the liquid, but the top remains open to the environment, without the liquid filling into the film mold. In embodiments, the immersing may include fully immersing the film mold containing the biodegradable surgical membrane into the liquid so that the liquid fills into the film mold.

In embodiments, the warming comprises: removing the film mold containing the biodegradable surgical membrane from the liquid and warming the film mold containing the biodegradable surgical membrane to room temperature over a time period of about 0.5-10 hours, or about 2 hours.

In embodiments, the method further comprises soaking the film mold in a distilled water ice bath which is allowed to warm to room temperature over a time period of about 1-3 hours, or about 2 hours, after removing the film mold containing the biodegradable surgical membrane from the liquid, and removing the film mold containing the biodegradable surgical membrane from the water bath.

In embodiments, the UV light has a wavelength in a range of about 100-420 nm, about 250-420, about 100-400 nm, about 250-400 nm, or about 250-365 nm, and an intensity of between about 25 microjoules (μJ) and 10M μJ.

In embodiments, the biodegradable surgical membrane has a thickness of about 0.1-50 mm, or about 0.1-10 mm, about 0.5-5 mm, or about 1.25 mm.

In embodiments, the biodegradable surgical membrane has a porosity of about 50-99% v/v and has micro-pores having an average pore size or average diameter of 0.1-100 μm.

In embodiments, the first polymer may comprise poly (L-lactic acid) (PLLA), poly-3-hydroxybutyrate (P3HB), poly(lactic-co-glycolic acid) (PLGA), a copolymer containing PLLA and having a bulk crystallinity of about 30-100%, or about 50-90%, or a combination thereof.

In embodiments, the second polymer may poly-ε-caprolactone diacrylate (PCL-DA), poly-ε-caprolactone triacrylate (PCL-TA), poly-4-hydroxybutyrate (P4HB), a copolymer containing poly-ε-caprolactone diacrylate (PCL-DA), poly(lactic-co-glycolic acid) diacrylate (PLGA-DA), poly(lactic-co-glycolic acid) triacrylate (PLGA-TA), or a combination thereof.

In embodiments, the radical initiator solution includes a radical initiator and the second organic solvent. In embodiments, the radical initiator is a photo initiator selected from the group of 2-hydroxy-4′-(2-hydroxyethoxy)-2-methylpropiophenone (such as Irgacure 2959) (excitation wavelength: 365 nm), 1-vinyl-2 pyrrolidinone (NVP, excitation wavelength: 365 nm), Lithium phenyl-2,4,6-trimethylbenzoylphosphinate (LAP, excitation wavelength: 405 nm), and a combination thereof.

In embodiments, the second organic solvent is selected from the group of methanol, tetrahydrofuran (THF), benzene, toluene, dioxane, methanol, ethanol, ethyl acetate, methylene chloride, and a mixture thereof.

In embodiments, the first organic solvent is selected from the group of tetrahydrofuran (THF), benzene, toluene, dioxane, methanol, ethanol, ethyl acetate, methylene chloride, and a mixture thereof. In embodiments, the first organic solvent is tetrahydrofuran (THF).

In embodiments, the first polymer has a molecular weight between 500 Da and 200 KDa before and after the reacting. In embodiments, the second polymer has an initial molecular weight between 500 Da and 200 KDa before the reacting and a final molecular weight between 20 KDa and 500 KDa after the reacting, and the final molecular weight is greater than the initial molecular weight of the second polymer.

In embodiments, the first polymer has a melting point of about 75° C. and about 200° C., and the second polymer has a melting point of about 45° C. and about 75° C.

In embodiments, the biodegradable surgical membrane exhibits bimodal melting points at about 40-75° C. and about 75-200° C. respectively. In embodiments, the biodegradable surgical membrane exhibits two distinct melting points at about 45-70° C. and about 80-200° C. respectively. In embodiments, the biodegradable surgical membrane exhibits two distinct melting points at about 48-55° C. and about 140-180° C. respectively. In embodiments, the biodegradable surgical membrane exhibits two distinct melting points at about 50-55° C. and about 170° C. respectively.

In embodiments, the biodegradable surgical membrane does not undergo a phase transition and does not absorb significant heat flow at a temperature in a range of about 37° C. and about 42° C.

In embodiments, the biodegradable surgical membrane is characterized by a first tensile modulus which remains substantially unchanged in a temperature range of about 25° C. to a temperature about 1-5° C. lower than the melting point of the second polymer before the reacting.

In embodiments, the biodegradable surgical membrane is characterized by a second tensile modulus at a temperature about 1-5° C. higher than the melting point of the second polymer, and the second tensile modulus is at least about 20%, or about 30-40% lower than the first tensile modulus.

In embodiments, the biodegradable surgical membrane has an original shape as fabricated and the biodegradable polymeric material maintains at least about 90% (95%, 90-100%) of the original shape after at least about 10 repeated cycles of heating, deforming and cooling the biodegradable surgical membrane, wherein the heating includes heating to a temperature above the melting point of the second polymer and below the melting point of the first polymer, wherein the deforming occurs at the temperature above the melting point of the second polymer and below the melting point of the first polymer, and wherein the cooling includes cooling to about 25° C.

In embodiments, the biodegradable surgical membrane maintains at least about 95% of the original shape after at least about 50 repeated cycles of the heating, deforming, and cooling.

In embodiments, the biodegradable surgical membrane has an air-inhibited layer and a porous layer, wherein the air-inhibited layer has a porosity less than about 40% v/v, less than about 30% v/v, less than about 20% v/v, or less than about 10% v/v, and the porous layer has a porosity higher than about 60% v/v, higher than about 70% v/v, higher than about 80% v/v, or higher than about 90% v/v.

In embodiments, the polymer-initiator solution in the film mold has an air-facing side in contact with air and a mold-facing side in contact with the bottom wall of the film mold, thereby providing the biodegradable surgical membrane having an air-facing side as an air-inhibited layer and a bottom film mold-facing side as a porous layer, wherein the air-inhibited layer has a porosity less than 40% v/v and is smooth, and the bottom porous layer has a porosity higher than 60% v/v and is porous.

In embodiments, the biodegradable surgical membrane has a biphasic morphology having an air-inhibited layer (smooth layer) and a porous layer.

In embodiments, the air-inhibited layer has a thickness of about 0.01-10 mm, or about 0.01-5 mm, and is less than about 50%, or is about 10-50%, about 5-25% or about 1-10% of the thickness of the biodegradable surgical membrane, and the porous layer has a thickness of about 0.1-10 mm and is greater than about 50%, about 70%, about 80%, about 90%, or about 99% of the thickness of the biodegradable surgical membrane.

In embodiments, the air-inhibited layer may be rich in the second polymer and may comprise greater than about 40%, about 50%, about 55%, about 60%, about 65%, or about 70% of the total amount of the second polymer. The weight ratio of the second polymer to the first polymer in the air-inhibited layer is higher than those of the porous layer or the biodegradable surgical membrane. The porous layer may be rich in the first polymer and may comprise greater than about 50%, about 55%, about 60%, about 65%, or about 70% of the total amount of the first polymer having the crystalline nanostructures. The weight ratio of the first polymer to the second polymer in the porous layer is higher than those of the air-inhibited layer or the biodegradable surgical membrane.

In embodiments, the first polymer is PLLA, the second polymer is PCL-DA, the air-inhibited layer comprises greater than about 50%, about 55%, about 60%, about 65%, or about 70% of the total amount of PCL-DA or coupled PCL-DA, the porous layer comprises greater than about 50%, about 55%, about 60%, about 65%, or about 70% of the total amount of the PLLA having the crystalline nanofibers.

In embodiments, the rate of degradation of the porous layer is about 1-10 times faster than the rate of degradation of the air-inhibited layer.

In embodiments, the cooling comprises applying a uniaxial temperature gradient to the polymer-initiator solution in the film mold.

In embodiments, the crystalline nanofibers are oriented parallel to the direction of the uniaxial temperature gradient or at an angle of about 0-25° from the direction of the uniaxial temperature gradient.

In embodiments, the direction of the uniaxial temperature gradient is a vertical direction perpendicular to the bottom wall of the film mold such that the polymer-initiator solution contacting the bottom wall of the film mold is at a lower temperature than the polymer-initiator solution at the top of the film mold, and the crystalline nanofibers are oriented parallel to the vertical direction or at an angle of about 0-25° from the vertical direction.

In embodiments, the cooling results in the polymer-initiator solution having a uniaxial temperature gradient in a vertical direction perpendicular to the bottom wall of the film mold, wherein the polymer-initiator solution contacting the bottom wall of the film mold is at a lower temperature than the polymer-initiator solution at the top of the film mold, such that the nanofibers are oriented in the vertical direction.

In embodiments, the first organic solvent is benzene, and the crystalline nanostructures are crystalline micro-tubes and/or nanotubes of the first polymer having an average diameter of about 0.01-500 μm.

In embodiments, the cooling may comprise applying a uniaxial temperature gradient to the polymer-initiator solution in the film mold in a vertical direction perpendicular to the bottom of the film mold, and wherein the crystalline microtubes and/or nanotubes are oriented parallel to the vertical direction or at an angle of about 0-25° from the vertical direction.

In embodiments, the biodegradable surgical membrane has an original shape as fabricated and maintains at least 95% of the original shape after at least about 50 repeated cycles of heating to a temperature of 45-75° C., deforming and shaping at the temperature of 45-75° C., and cooling of the biodegradable surgical membrane.

In embodiments, the crystalline nanofibers are present in the biodegradable surgical membrane after at least about 50 repeated cycles of heating to a temperature up to 75° C., deforming and shaping at the temperature up to 75° C., and cooling to room temperature.

In embodiments, the microtubes are present in the biodegradable surgical membrane after at least about 50 repeated cycles of heating to a temperature up to 75° C., deforming and shaping at the temperature up to 75° C., and cooling to room temperature.

In embodiments, the first polymer may comprise poly (L-lactic acid) (PLLA) and the second polymer may comprise poly-ε-caprolactone diacrylate (PCL-DA).

In embodiments, the first polymer may comprise poly (L-lactic acid) (PLLA), the second polymer may comprise poly-ε-caprolactone diacrylate (PCL-DA) or poly-ε-caprolactone triacrylate (PCL-TA), and the third polymer may comprise PLGA-DA or PLGA-TA. The first, may be present in the membrane in an amount of about 50-95 wt. %, or about 60-90 wt. % by weight of the membrane; and the total amount of the second and third polymers is in a range of about 5-50 wt. %, or about 10-40 wt. % by weight of the membrane.

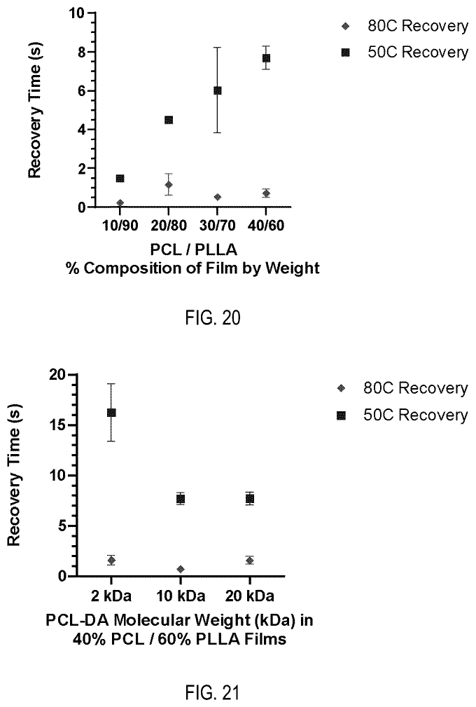

Applicant surprisingly found that the amount of the first polymer (linear polymer capable of forming crystalline nanostructures and having higher Tm) should be at least about 50 wt. %, and preferably at least about 60 wt. % by weight of the membrane or scaffold for the first polymer to form crystalline nanostructures and partially phase separate from the rest of the polymer networks in microscale to retain the physical properties at near physiologic temperatures and further to have the shape memory properties, as detailed in Example 7 and as shown in FIGS. 10B-10F. Applicant further surprisingly found that the amount of the second polymer (polymer having lower Tm) should be at least 5 wt. %, and preferably at least 10 wt. % by weight of the membrane or scaffold for the second polymer to act as a thermosensitive switch for the membrane to have the shape memory properties, as detailed in Example 8 and as shown in FIGS. 20 and 21.

In embodiments, the biodegradable surgical membrane is a periodontal membrane.

Another aspect of the present disclosure provides a biodegradable surgical membrane comprising: a first polymer and a second polymer in a weight ratio of between about 50:50 and about 99:1, the first polymer being a biodegradable linear polymer having first polymer chains and a crystallinity of about 10-100% or about 20-80%; the second polymer being a biodegradable polymer and having second polymer chains each of which has two or more functional groups, at least a portion of the second polymer being coupled with each other through the two or more functional groups, wherein the biodegradable surgical membrane has an interpenetrating polymer network (IPN) or semi-interpenetrating polymer network (SIPN) between the first and coupled second polymer chains, and wherein the second polymer chains are interspersed throughout the first polymer chains.

In embodiments, the biodegradable surgical membrane may comprise a third polymer having two or more functional groups. In embodiments, biodegradable surgical membrane may comprise a compound having two or more functional groups.

The selection, properties, and interaction/reaction of the first, second and/or third polymers, and/or the compound are described herein above.

In embodiments, the biodegradable surgical membrane has a biphasic morphology including: a top smooth layer having a porosity no more than about 40% v/v; and a bottom porous layer having a porosity of at least about 60% v/v. In embodiments, the biodegradable surgical membrane may have a homogeneous porosity morphology without layers of different porosity.

In embodiments, the biodegradable surgical membrane has a thickness of 0.1-10 mm, the top smooth layer has a thickness of 0.01-5 mm, and the bottom porous layer has a thickness of 0.1-9.99 mm.

In embodiments, the bottom porous layer has micro-pores having an average pore size or average diameter of about 0.01-100 μm.

In embodiments, the rate of degradation of the bottom porous layer is about 1-10 times faster than the rate of degradation of the top smooth layer.

In embodiments, the second polymer may be at least partially crosslinked and may have a crosslinking density of about 0.1-50% or about 1-20%. In embodiments, the second polymer has two functional groups, the biodegradable surgical membrane may comprise a third polymer having third polymer chains each of which has three or more functional groups, and the third polymer is present in an amount of 0.001-20 wt. % or about 0.1-10 wt. % of the biodegradable surgical membrane. In embodiments, the third polymer may be at least partially crosslinked with each other and/or with the second polymer chains. In embodiments, the biodegradable surgical membrane may have the IPN or SIPN of the first polymer chains and the coupled second and third polymer chains, and the second and third polymer chains are interspersed throughout the first polymer chains.

In embodiments, the first polymer may be poly (L-lactic acid) (PLLA) and the second polymer may be poly-ε-caprolactone diacrylate (PCL-DA) and/or poly-ε-caprolactone triacrylate (PCL-TA).

In embodiments, the biodegradable surgical membrane is a periodontal membrane.

Another aspect of the present disclosure provides a method for providing a dental implant to a subject in need thereof, the method comprising: implanting a periodontal membrane comprising the biodegradable surgical membrane to a defective dental site in a subject.

In embodiments, the periodontal membrane has an air-inhibited layer and a porous layer, wherein the air-inhibited layer has a porosity less than about 50% v/v, less than about 40% v/v, less than about 30% v/v, less than about 20% v/v, or less than about 10% v/v and is smooth, and wherein the porous layer has a porosity higher than about 50% v/v, higher than about 60% v/v, higher than about 70% v/v, higher than about 80% v/v, higher than about 90% v/v, or about 90-99%, and is porous. In embodiments, the periodontal membrane may be implanted such that the air-inhibited layer is on top and in contact with the air and the porous layer is on bottom and in contact with the defective dental site.

In embodiments, the air-inhibited layer substantially prevents tissue growth on the air-inhibited layer of the periodontal membrane, and the porous layer of the biodegradable surgical membrane facilitates tissue growth and augment cellular regenerative processes on the bottom porous layer of the periodontal membrane after the dental implant.

In embodiments, the implanting may include fixing the periodontal membrane to the defective dental site by sutures, screws, and/or adhesives. In embodiments, the fixing comprises fixing the periodontal membrane to the defective dental site by a suture. In embodiments, the suture may be retained in the periodontal membrane for a time period of at least about 10 days after the periodontal membrane is fixed to the defective dental site.

In embodiments, the biodegradable surgical membrane has crystalline nanofibers of the first polymer and maintains the crystalline nanofibers during the implantation of the dental implant.

In embodiments, the biodegradable polymeric material is a biodegradable surgical membrane having a thickness of about 0.01-10 mm.

In embodiments, the biodegradable surgical membrane has a biphasic morphology including: a top smooth layer having a porosity less than about 50% v/v, less than about 40% v/v, less than about 30% v/v, less than about 20% v/v, or less than about 10% v/v; and a bottom porous layer having a porosity higher than about 50% v/v, higher than about 60% v/v, higher than about 70% v/v, higher than about 80% v/v, higher than about 90% v/v, higher than about 95% v/v, higher than about 98% v/v, or about 90-99% v/v, wherein the top smooth layer has a thickness of about 0.01-5 mm, and the bottom porous layer has a thickness of about 0.1-10 mm, and wherein the bottom porous layer has micro-pores having an average pore size or average diameter of about 0.01-100 μm, or about 1-100 μm.

Another aspect of the present disclosure provides a method for making a macroporous tissue engineering scaffold, the method comprising: admixing a polymer mixture comprising a first polymer and a second polymer in a weight ratio between about 50:50 and about 99:1, the first polymer being a biodegradable linear polymer and having first polymer chains, the second polymer being a biodegradable polymer and having second polymer chains each of which includes two or more reactive functional groups; and a first organic solvent to form a polymer mixture solution, wherein the polymer mixture solution comprises about 1-30 wt. % of the polymer mixture by weight of the polymer mixture solution and about 70-99 wt. % of the first organic solvent by weight of the polymer mixture solution; admixing a reagent solution with the polymer mixture solution to form a polymer-reagent solution; combining in a container the polymer-reagent solution and a sugar porogen scaffold template comprising sugar particles; reacting the polymer-reagent solution to form coupled second polymer chains, thereby forming an initial scaffold including the sugar porogen scaffold template, wherein reacting includes coupling (such as chain-extending and/or at least partially crosslinking) the second polymer chains with each other through the two or more functional groups; cooling the initial scaffold; and dissolving the sugar particles in the initial scaffold thereby forming the macroporous tissue engineering scaffold, wherein the macroporous tissue engineering scaffold has an interpenetrating polymer network (IPN) or a semi-interpenetrating polymer network (SIPN) between the first polymer chains and the coupled second polymer chains, and wherein the second polymer chains are interspersed throughout the first polymer chains.

In embodiments, the reagent may be a radical initiator, a catalyst, a crosslinking agent, or a combination thereof. In embodiments, the reagent is a radical initiator, and the polymer-reagent solution is a polymer-initiator solution. In embodiments, the radical initiator is a photo initiator, and the reacting includes irradiating the polymer-initiator solution with high intensity UV lights to form the initial scaffold including the sugar porogen scaffold template.

In embodiments, the radical initiator is a photo initiator selected from the group of 2-hydroxy-4′-(2-hydroxyethoxy)-2-methylpropiophenone (such as Irgacure 2959) (excitation wavelength: 365 nm), 1-vinyl-2 pyrrolidinone (NVP, excitation wavelength: 365 nm), Lithium phenyl-2,4,6-trimethylbenzoylphosphinate (LAP, excitation wavelength: 405 nm), and a combination thereof.

In embodiments, each of the second polymer chains may include two functional groups. In embodiments, each of the second polymer chains may include two terminus and a terminal functional group at each of the two terminus. In embodiments, the polymer mixture may include a third polymer having three or more reactive functional groups which are reactive or non-reactive with the functional groups of the second polymer. In embodiments, the reacting including coupling (including chain extending and at least partially crosslinking) the third polymer chains with each other and/or with the second polymer chains, and the macroporous scaffold has the IPN or SIPN between the first polymer chains and the coupled second and third polymer chains. The coupled second and third polymer chains may be homogeneously interspersed throughout the first polymer chains. In embodiments, the second polymer may have a crosslinking density of about 1-20%. In embodiments, the second and third polymers may have a crosslinking density of about 1-20%.

In embodiments, the polymer mixture may comprise a compound having two or more functional groups.

The selection, properties, and interaction/reaction of the first, second and/or third polymers, and/or the compound are described herein above.

In embodiments, the sugar particles are in a shape including spheres, cubes, cylinders, cones, rectangular prisms, triangular prisms, or a combination thereof. The sugar particles may be in any other suitable shapes.

In embodiments, the sugar particles are in the shape of spheres, the sugar particles are sugar spheres, and the sugar porogen scaffold template is a sugar sphere porogen scaffold template comprising the sugar spheres.

In embodiments, the sugar spheres are sugar microspheres having an average particle size or average diameter of about 0.1-1000 μm, about 10-500 μm, or about 30-450 μm.

In embodiments, the radical initiator solution comprises a radical initiator and a second organic solvent, and the radical initiator has a concentration of about 0.01-50 mM, about 1-10 mM, or about 3 mM in the radical initiator solution, and a weight ratio of the initiator to the polymer mixture is about 0.01-20 wt. %, 0.1-10 wt. %, or about 0.1-5 wt. %.

The terms “air-inhibited layer”, “air-inhibited smooth layer”, “top smooth layer”, and “smooth layer” are used interchangeably herein, unless specified otherwise. The terms “porous layer” and “bottom porous layer” are used interchangeably herein, unless specified otherwise.

In embodiments, the radical initiator solution is admixed with the polymer mixture solution in a volume ratio of about 0.1:100 to about 50:100 v/v, about 1:100 to about 30:100 v/v, or about 10:100 v/v.

In embodiments, the second polymer chains in the macroporous tissue engineering scaffold are coupled to each other through the two or more functional groups and are at least partially crosslinked with each other and has a crosslinking density of about 1-20%.

In embodiments, the first polymer in the macroporous tissue engineering scaffold is at least partially phase separated from the crosslinked second polymer, the first polymer having a crystallinity of about 20-80%, and about 20-80 wt. % of the first polymer is in the form of crystalline nanostructures.

In embodiments, the method comprises heating the polymer-initiator solution to a temperature of about 40-90° C., or about 50-80° C.

In embodiments, the method further comprises cooling the macroporous tissue engineering scaffold at a temperature between about 0° C. and about −120° C. for about 1-60 hours, for example, 6 hours, 12 hours, 24 hours, 36 hours, 48 hours, or 60 hours.

In embodiments, the first polymer may have a molecular weight between 500 Da and 200 KDa before and after the reacting. In embodiments, the second or third polymer may have an initial molecular weight between 500 Da and 200 KDa before the reacting, and a final molecular weight of about between 20 KDa and 500 KDa after the reacting, and the final molecular weight is greater than the initial molecular weight of the second or third polymer.

In embodiments, the UV light has a wavelength in a range of about 100-420 nm, about 250-420, about 100-400 nm, about 250-400, or about 250-365 nm, and an intensity in a range of 25 to 10M microjoules (p J).

In embodiments, the macroporous tissue engineering scaffold has a porosity of about 50-99% v/v, about 60-99% v/v, about 70-99% v/v, about 80-99% v/v, or about 90-99% v/v. In embodiments, the macroporous tissue engineering scaffold has macropores having an average pore size or average diameter of about 0.1-1000 μm, about 1-500 μm, or about 30-450 μm.

In embodiments, the container has a bottom wall and four side walls, and the container does not have a cover and is thus open to the environment. The resulting scaffold may form bi-layer morphology with an air-inhibited smooth layer having a porosity less than about 50% v/v, or less than about 40% v/v, or less than about 30% v/v, or less than about 20% v/v, or less than about 10% v/v, and a porous layer having a porosity higher than about 60% v/v, or about 70% v/v, or about 90% v/v, or about 90% v/v, or about 95% v/v, when the film mold is open to the environment. The smooth layer is substantially free of pores, wherein substantially free refers to a porosity of less than about 10%, or about 5%, or about 2%. The smooth layer further has no or substantially no macropores. The porous layer has macropores having average pore size of about 1-500 μm, or about 30-450 μm.

In embodiments, the container has a bottom wall and four side walls, and the container has a cover and is thus closed to the environment. The resulting scaffold may have a homogeneous porosity morphology without layers of differing porosity when the film mold is closed to the environment.

In embodiments, the first polymer may comprise poly (L-lactic acid) (PLLA); comprise poly-3-hydroxybutyrate (P3HB); poly(lactic-co-glycolic acid) (PLGA); a copolymer containing PLLA and having a bulk crystallinity of about 10-100%, or about 50-80%; or a combination thereof. The first polymer may be modified to have two or more functional groups.

In embodiments, the second polymer may comprise poly-ε-caprolactone diacrylate (PCL-DA), poly-ε-caprolactone triacrylate (PCL-TA), a copolymer containing PCL-DA or PCL-TA, poly-4-hydroxybutyrate (P4HB) having two or more functional groups, PLGA-DA, PLGA-TA, or a combination thereof.

In general, the first organic solvent can be any organic solvent suitable to completely dissolve the polymer mixture which includes the first, second and/or third polymers and/or the compound. Applicant surprisingly found that the selection of the first organic solvent impacts the microstructures of the resulting membrane or scaffold, especially the crystalline structures of the first polymer in either the membranes or scaffolds. The first organic solvent selection depends on the desirable morphology of the resulting membranes or scaffolds. If crystalline nanostructures (such as nanofibers or nanotubes) or microtubes of the first polymer are desirable, the solvent having the ability to induce thermally induced phase separation of the nanostructures of the first polymer should be selected. These solvents include tetrahydrofuran (THF), benzene, toluene, dioxane, methanol, ethanol, ethyl acetate, methylene chloride, and a mixture thereof. These solvents may induce thermally induced phase separation of the crystalline structures (such as nanofibers, nanotubes or microtubes) of the first polymer from the amorphous second and/or third polymers. On the contrary, if crystalline nanostructures (such as nanofibers or nanotubes) or microtubes of the first polymer are not desirable, but rather smooth membrane or scaffold are desirable, then additional solvents, such as ethyl acetate and methylene chloride would be suitable.

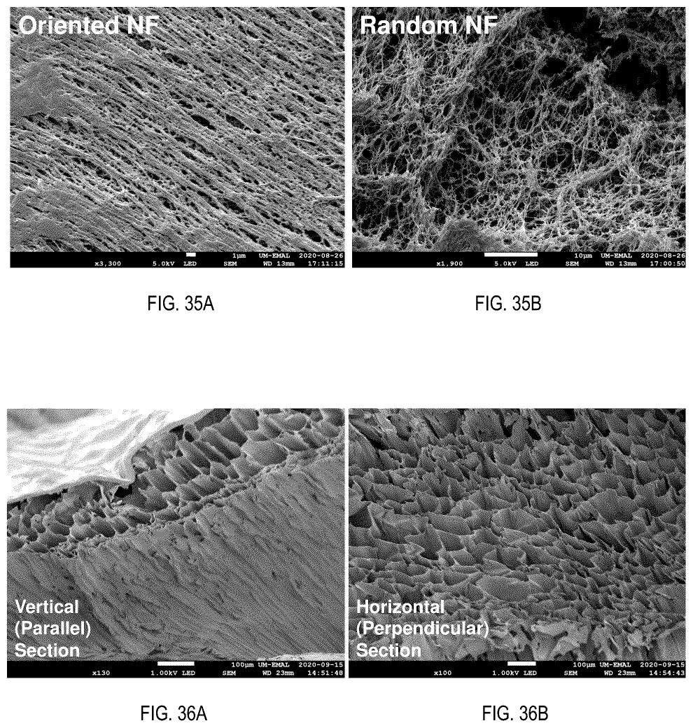

Applicant further surprisingly found that the selection of the first organic solvent can also impact the crystalline structures of the first polymer in either membranes or scaffolds. Applicant found that when the first organic solvent is tetrahydrofuran (THF), the resulting crystalline nanostructures of the first polymer are crystalline nanofibers which may have a density and spacing in a range of 2.5-20% wt/v of the macroporous tissue engineering scaffold. For example, as detailed in Example 6, when the first organic solvent was THF, the first polymer PLLA formed crystalline nanofibers, as shown in FIGS. 10B-E. Applicant further found that when the first organic solvent is benzene, the resulting crystalline nanostructures are crystalline microtubes and/or nanotubes of the first polymer. For example, as detailed in Example 12, when the first organic solvent was changed to benzene, the thermally induced phase separation of the first polymer PLLA from benzene in a TS-MMS composite (5% wt/v, for example) formed the oriented crystalline microtubes and/or crystalline nanotubes, as shown in FIGS. 36A and 36B.

Based on Applicant's findings that the first organic solvent can impact the microstructures of the resulting membrane or scaffold, especially the crystalline structures of the first polymer in either membranes or scaffolds, the first organic solvent can thus be selected to control the microstructures of the crystalline first polymer in either the membranes or the scaffolds.

In embodiments, the first organic solvent is selected from the group of tetrahydrofuran (THF), benzene, toluene, dioxane, methanol, ethanol, ethyl acetate, methylene chloride, and a mixture thereof. In embodiments, the first organic solvent is selected from the group of ethyl acetate, methylene chloride and a mixture thereof. In embodiments, the first organic solvent is THF. In embodiments, the first organic solvent is benzene.

In embodiments, the second organic solvent is selected from the group of methanol, tetrahydrofuran (THF), benzene, toluene, dioxane, ethanol, ethyl acetate, methylene chloride, and a mixture thereof. In general, the second organic solvent can be any organic solvent suitable to completely dissolve the reagent. The second organic solvent may also need to be miscible with the first organic solvent. In embodiments, the second solvent is methanol.

In embodiments, the cooling of the macroporous tissue engineering scaffold comprises applying a uniaxial temperature gradient to the macroporous tissue engineering scaffold, thereby forming the crystalline nanofibers oriented parallel to direction of the uniaxial temperature gradient or at an angle of about 0-25° from the direction of the uniaxial temperature gradient.

In embodiments, the macroporous tissue engineering scaffold may exhibit two distinct melting points at about 40-75° C. and about 75-200° C. respectively. In embodiments, the macroporous tissue engineering scaffold exhibits bimodal melting points having a first melting point at about 45-70° C. and se second melting point at about 80-190° C.

In embodiments, the macroporous tissue engineering scaffold may not undergo a phase transition (does not absorb significant heat flow) in a temperature range of about 37° C. and about 42° C.

In embodiments, the macroporous tissue engineering scaffold has an air-inhibited layer having a porosity of less than about 50%, less than about 40%, less than about 30%, less than about 20%, or less than about 10%, and a porous layer having a porosity greater than about 50%, greater than about 60%, greater than about 70%, greater than about 80%, greater than about 90%, or about 90-99%, and wherein the air-inhibited layer is substantially free of macro-pores, and the bottom porous layer includes macropores.

In embodiments, the macroporous tissue engineering scaffold is characterized by a first tensile modulus which is maintained in a temperature range of about 25° C. to a temperature about 1-5° C. lower than the melting point of the second polymer.

In embodiments, the macroporous tissue engineering scaffold is characterized by a second tensile modulus at a temperature about 1-5° C. higher than the melting point of the second polymer, wherein the second tensile modulus is at least about 20% lower than the first tensile modulus.

In embodiments, macroporous tissue engineering scaffold has an original shape as fabricated and the macroporous tissue engineering scaffold maintains at least about 90% of the original shape after at least about 10 repeated cycles of heating, deforming, and cooling the macroporous tissue engineering scaffold, wherein the heating includes heating to a temperature above the melting point of the second polymer and below the melting point of the first polymer, deforming occurs at the temperature above the melting point of the second polymer and below the melting point of the first polymer, and cooling includes cooling to room temperature.

In embodiments, the air-inhibited layer of the macroporous tissue engineering scaffold comprises at least 50% of the total amount of the second polymer (PCL-DA), the porous layer comprises at least 50% of the total amount of the first polymer (PLLA) and the crystalline nanofibers. In embodiments, the rate of degradation of the air-inhibited layer is about 1-10 times slower than the rate of degradation of the porous layer.

A non-limiting example is that the macroporous tissue engineering scaffold is for a dental implant. After implanting the macroporous tissue engineering scaffold to a defective dental site of a subject, the porous layer of the macroporous tissue engineering scaffold degrades at a degradation rate 1-10 times faster than the air-inhibited layer does in the oral environment of the subject.

In embodiments, the method further comprises applying a uniaxial temperature gradient to the polymer-initiator solution in the container in a vertical direction perpendicular to the bottom of the container, thereby providing the crystalline microtubes and/or nanotubes in an orientation parallel to the vertical direction or at an angle of about 0-25° from the vertical direction.

In embodiments, the first polymer is poly (L-lactic acid) (PLLA), and the second polymer is poly-ε-caprolactone diacrylate (PCL-DA) and/or poly-ε-caprolactone triacrylate (PCL-TA).

In embodiments, the macroporous tissue engineering scaffold is a periodontal scaffold for a dental implant.

Another aspect of the present disclosure provides a method for providing a dental implant to a subject in need thereof, the method comprising: implanting the macroporous tissue engineering scaffold made by the method discussed herein above or elsewhere in the present disclosure to a defective dental site in the subject. In embodiments, the implanting includes fixing the macroporous tissue engineering scaffold to the defective dental site by sutures, screws, and/or adhesives. In embodiments, the macroporous tissue engineering scaffold is fixed to the defective dental site by a suture.

In embodiments, the macroporous tissue engineering scaffold has crystalline nanofibers of the first polymer and the crystalline nanofibers are maintained during the implantation of the dental implant.

In embodiments, the crystalline nanofibers are maintained in the macroporous tissue engineering scaffold after at least 50 repeated cycles of heating a temperature up to about 75° C., deforming and shaping at the temperature up to about 75° C., and cooling to room temperature.

In embodiments, the macroporous tissue engineering scaffold has an air-inhibited layer and a porous layer, wherein the air-inhibited layer has a porosity less than about 40% v/v and is smooth and substantially free of macro-pores, and the porous layer has a porosity greater than about 60% v/v and comprises macro-pores, and wherein the air-inhibited layer substantially prevents tissue growth and the porous layer facilitates tissue growth and augment cellular regenerative processes after implantation of the dental implant.

Another aspect of the present disclosure provides a macroporous tissue engineering scaffold comprising: a first polymer and a second polymer in a weight ratio of between about 50:50 and about 99:1, the first polymer being a biodegradable linear polymer and having first polymer chains, the second polymer being a biodegradable polymer, the second polymer having second polymer chains each of which has two or more functional group, at least a portion of the second polymer being coupled (such as chain extending and/or at least partially crosslinking) with each other through the two or more functional groups, wherein the macroporous tissue engineering scaffold has an IPN or SIPN between the first and coupled second polymer chains wherein the second polymer chains are interspersed throughout the first polymer chains, wherein the first polymer is at least partially crystallized and at least partially phase separated from the coupled second polymer, the first polymer has a crystallinity of 20-80%, and the first polymer having crystalline nanostructures homogeneously dispersed in the interpenetrating polymer network (IPN), and wherein the macroporous tissue engineering scaffold has a porosity of about 50-99% v/v or about 90-98% v/v and has macro-pores having an average pore size or average diameter/pore size of about 1-1000 μm or about 30-450 μm. In embodiments, the second polymer may be at least partially crosslinked and may have a crosslinking density of about 0.1-50% or about 1-20%. In embodiments, the second polymer has two functional groups, the macroporous scaffold has a third polymer having three or more functional groups, the IPN or SIPN has the first and coupled second and third polymer chains interpenetrating through each other, and the coupled second and third polymer chains are interspersed throughout the first polymer chains.

Another aspect of the present disclosure provides a method of preparing a system comprising a macroporous tissue engineering scaffold and a controlled release composition embedded in the scaffold, the method comprising: combining a polymer mixture comprising a first polymer and a second polymer in a weight ratio in a range between about 50:50 and about 99:1, the first polymer being a biodegradable linear polymer and having first polymer chains, the second polymer being a biodegradable polymer and having second polymer chains each of which includes two or more functional groups; and a first organic solvent to form a polymer mixture solution having 1-30 wt. % of the first and second polymers and 70-99 wt. % of the first organic solvent by weight of the polymer mixture solution; admixing a reagent (such as a radical initiator) solution with the polymer mixture solution in a volume ratio of between about 0.1:100 v/v and about 20:1 v/v, or between about 1:100 v/v and about 10:1 v/v, to form a polymer-reagent (such as polymer-initiator) solution, the reagent solution comprising the reagent and a second organic solvent and having a concentration of about 0.01-100 mM, about 0.1-50 mM, about 1-10 mM, or about 3 mM of reagent in the reagent solution; combining in a container the polymer-reagent solution and a sugar porogen scaffold template, the sugar porogen scaffold template comprising sugar particles and nanoparticles having the controlled release composition, and the nanoparticles attached to a surface of the sugar particles; reacting the polymer-reagent solution in the container to form coupled second polymer chains, thereby forming an initial scaffold including the sugar porogen scaffold template, wherein the reacting includes coupling (including chain-extended and/or at least partially crosslinked) the second polymer chains in a matrix of the first polymer to form an IPN or SIPN between the first and coupled second polymer chains; and dissolving (leaching) the sugar spheres with water to form the system. In embodiments, the reagent solution may comprise a reagent and a second organic solvent. In embodiments, the reagent is a radical initiator, a catalyst, a crosslinking agent, or a combination thereof.

In embodiments, the second polymer may be chain-extended. The second polymer may be partially crosslinked and have a crosslinking density of about 0.1-50% or about 1-20%. In embodiments, the reagent is a radical initiator, the polymer-reagent solution is a polymer-initiator solution, and the reacting includes irradiating the polymer-initiator solution in the container with a UV light to form an initial scaffold including the sugar porogen scaffold template. The reacting may include coupling the second polymer chains with each other. The coupling may include chain-extending and/or at least partially crosslinking the second polymer chains.

In embodiments, the second polymer may have two functional groups and the polymer mixture may comprise a third polymer having three or more functional groups. The reacting may include coupling the second and third polymers. The coupling may include chain-extending the second and third polymer chains respectively and/or with each other; and/or at least partially crosslinking the third polymer chains with each other and/or with the second polymer chains. In embodiments, the polymer mixture may comprise a compound having two or more functional groups.

The selection, properties, and interaction/reaction of the first, second and/or third polymers, and/or the compound are described herein above.

The properties and methods of making the sugar particles are discussed herein above and include sugar spheres.

In embodiments, the sugar sphere porogen scaffold template comprises the sugar spheres and nanoparticles of the controlled release composition, wherein the nanoparticles are attached to a surface of the sugar spheres and the nanoparticles comprise a substrate and a first drug substance, wherein the dissolving of the sugar spheres does not dissolve the nanoparticles and the nanoparticles remain in the macroporous tissue engineering scaffold after leaching out the sugar particles.

In embodiments, the nanoparticles are homogeneously dispersed and embedded in the macroporous tissue engineering scaffold.

In embodiments, the method comprises preparing the sugar sphere porogen scaffold template by a process comprising: admixing a nanoparticle suspension comprising (i) the nanoparticles having the substrate and the first drug substance, (ii) a first surfactant, and (iii) a third organic solvent, wherein the nanoparticles are homogeneously dispersed in the third organic solvent; and a sugar sphere slurry comprising the sugar spheres and the third organic solvent; to form a dispersion having solid particles homogeneously dispersed in the third solvent, the solid particles comprising the nanoparticles and the sugar spheres wherein the nanoparticles are attached to a surface of the sugar spheres; adding the dispersion to a Teflon mold; heat treating the dispersion to cause partial annealing of the sugar spheres for about 5-15 minutes; and removing the third solvent by vacuum evaporation to yield the sugar sphere porogen scaffold template.

In embodiments, the substrate is selected from the group of a fourth polymer, silica, and a combination thereof. In embodiments, the substrate comprises the fourth polymer selected from the group of 50:50 poly (D, L-lactide-co-glycolide) (PLGA), Poly (hydroxyethyl methacrylate) (poly(HEMA)) and copolymers of HEMA, polystyrene (PS), Polyethylene glycol (PEG) and copolymers of PEG, Poly(glycerol adipate) (PGA), Poly(methyl acrylate) (PMA) and copolymers of PMA, Poly acrylic acid (PAA) and copolymers of PAA, and a combination thereof. In embodiments, the fourth polymer is 50:50 poly (D, L-lactide-co-glycolide) (PLGA) having a molecular weight between about 7 kDa and about 17 kDa.

In embodiments, wherein the third organic solvent is selected from the group consisting of hexane, ethanol, methanol, acetonitrile, DMSO, DMF and a combination thereof.

In embodiments, the first drug substance is selected from the group of a small molecule, simvastatin, ibuprofen, a peptide, a peptide mimicking a growth factor, a modified peptide, a peptide conjugated to an affinity ligand, a growth factor, PDGF-bb, BMP-2, BMP-3, BMP-7, GDF-5/BMP-14, GDF-7/BMP-12, FGF-2, IGF-1, a growth factor fragment, PDGF-bb, BMP-2, BMP-3, BMP-7, GDF-5/BMP-14, GDF-7/BMP-12, FGF-2, IGF-1, a growth factor conjugated to an affinity ligand, PDGF-bb, BMP-2, BMP-3, BMP-7, GDF-5/BMP-14, GDF-7/BMP-12, FGF-2, IGF-1, a nanoparticle which directly displays therapeutic properties or therapeutic modalities, an exosome or extracellular vesicle, a polynucleic acid and/or gene delivery, an antibody Fc fragment, a monoclonal antibody, and a combination thereof.

In embodiments, the method comprises preparing the nanoparticles by a process comprising: sonicating and admixture of (a) a first drug substance mixture comprising the first drug substance and water; and (b) a fourth polymer solution comprising the fourth polymer (PLGA) and a fourth organic solvent (dichloromethane) to form a sonicated polymer-drug solution; admixing the sonicated polymer-drug solution with a fifth solvent (20 mL of 1% polyvinyl alcohol in double-distilled water (ddH₂O)) and sonicating to form a nanoparticle suspension; and ultra-centrifuging the nanoparticles suspension to yield the nanoparticles.

In embodiments, the process further comprises washing and drying the nanoparticles.

In embodiments, the fourth solvent is dichloromethane, and the fifth solvent is 1% polyvinyl alcohol (PVA) aqueous solution having 1 wt. % of PVA dissolved in water or sterile, nuclease-free water (ddH₂O).

In embodiments, the method further comprises preparing the sugar spheres by a process comprising: emulsifying molten D-Fructose in a mineral oil with Sorbitane monooleate (Span80) surfactant to form an emulsion; and quenching the emulsion by dropping the emulsion into a cold oil to form sugar droplets.

In embodiments, the method further comprises size selecting the sugar droplets by molecular sieves to form the sugar spheres having predetermined particle sizes. In embodiments, the predetermined particle sizes are about 1-1000 μm, about 10-500 μm, or about 30-450 μm.

In embodiments, the macroporous tissue engineering scaffold is for a dental implant. In embodiments, the macroporous tissue engineering scaffold having the nanoparticles releases the first drug substance at a controlled release rate in an oral environment of a subject, the controlled release rate determined by a degradation rate of the macroporous tissue engineering scaffold and/or a degradation rate of the fourth polymer in the oral environment.

In embodiments, the macroporous scaffold having the controlled release composition may have a porosity of about 50-99% or about 90-98% v/v and macro-pores having an average pore size or diameter of about 0.1-1000 μm, or about 30-450 μm.

In embodiments, the second polymer is at least partially crosslinked through the two or more functional groups, and has a crosslinking density of about 0.1-50% or about 1-20%. In embodiments, the polymer mixture may comprise a third polymer having third polymer chains each of which has three or more functional groups which may be reactive or non-reactive with the functional groups of the second polymer. The reacting may include (i) coupling (a) at least a portion of the second polymer chains to each other; (b) at least a portion of the second polymer chains to at least a portion of the third polymer chains; and/or (c) at least a portion of the third polymer chains to each other, to thereby chain extend at least a portion of the second polymer chains and/or third polymer chains; and/or (ii) partially crosslinking (a) at least a portion of the second polymer chains to each other; (b) at least a portion of the second polymer chains to at least a portion of the third polymer chains; and/or (c) at least a portion of the third polymer chains to each other, to thereby form the coupled second and third polymer chains. The scaffold comprises an IPN of the first, second and third polymer chains, and the coupled second and third polymer chains are interspersed throughout the first polymer chains.

In embodiments, the polymer mixture may comprise a compound having two or more functional groups which can react with the functional groups of the second polymer chains to form coupled second polymer chains. The reacting may comprise admixing the second polymer with the compound to thereby extend and/or partially crosslink at least a portion of the second polymer chains by coupling one or more second polymer chains with the compound and form the coupled second polymer chains including extended and/or crosslinked second polymer chains. The resulting scaffold may comprise an IPN or SIPN of the first polymer chains and the coupled second polymer chains. The coupled second polymer chains are interspersed throughout the first polymer chains.

In embodiments, the crystalline nanostructures are crystalline nanofibers of the first polymer, and the crystalline nanofibers are about 20-80 wt. % of the first polymer and have a density and spacing between 2.5-20% wt/v of the macroporous tissue engineering scaffold.

In embodiments, the crystalline nanofibers are oriented parallel to each other or at an angle of about 0-25° from an axis of the macroporous tissue engineering scaffold.

In embodiments, the macroporous tissue engineering scaffold may exhibit bimodal melting points, with a first melting point at about 45-75° C., or about 50-55° C., and a second melting point at about 75-200° C. or about 170° C. respectively. In embodiments, the first polymer has a melting point between about 75 and about 200° C., and the second polymer has a melting point between about 45 and about 75° C.

In embodiments, the macroporous tissue engineering scaffold is characterized by a first tensile modulus which is maintained in a temperature range of about 25° C. to a temperature about 1-5° C. lower than the melting point of the second polymer.

In embodiments, the macroporous tissue engineering scaffold characterized by a second tensile modulus at a temperature about 1-5° C. higher than the melting point of the second polymer, wherein the second tensile modulus is at least about 20% lower than the first tensile modulus.

In embodiments, macroporous tissue engineering scaffold has an original shape as fabricated and the macroporous tissue engineering scaffold maintains at least about 90% of the original shape after at least about 10 repeated cycles of heating, deforming, and cooling the macroporous tissue engineering scaffold, wherein the heating includes heating to a temperature above the melting point of the second polymer and below the melting point of the first polymer, deforming occurs at the temperature above the melting point of the second polymer and below the melting point of the first polymer, and cooling includes cooling to room temperature.

In embodiments, the macroporous tissue engineering scaffold has a biphasic morphology including: a smooth layer having a porosity less than about 40% v/v; and a porous layer having a porosity greater than about 90% v/v.

The selections of the first, second and third polymers are the same as those of the macroporous scaffold discussed herein above.

In embodiment, the system comprising the macroporous scaffold and the controlled release composition embedded in the macroporous scaffold is for a dental implant application. The method for providing a dental implant to a subject in need thereof may comprise: implanting the system to a defective dental site in the subject.

In embodiments, the implanting includes fixing the system having the macroporous tissue engineering scaffold and the controlled release composition to the defective dental site by sutures, screws, and/or adhesives.

In embodiments, the macroporous tissue engineering scaffold may be fixed to the defective dental site by a suture.

In embodiments, the macroporous tissue engineering scaffold may have crystalline nanofibers of the first polymer and the crystalline nanofibers may be maintained during the implementation of the dental implant. In embodiments, the crystalline nanofibers may remain in the macroporous tissue engineering scaffold after at least 50 repeated cycles of heating up to a temperature of about 75° C., deforming and shaping at the temperature up to about 75° C., and cooling.

In embodiments, the macroporous tissue engineering scaffold may have an air-inhibited layer having a porosity of less than about 40% v/v and a porous layer having a porosity greater than about 90% v/v, and wherein the air-inhibited layer substantially prevents tissue growth and the bottom porous layer facilitates tissue growth and augment cellular regenerative processes.

In embodiments, the method comprises cooling the initial scaffold, wherein the first polymer is at least partially phased separated from the coupled second polymer and at least partially crystallized to form crystalline nanostructures, the first polymer having a crystallinity of about 20-80%; dissolving the sugar spheres, wherein the dissolving does not remove the nanoparticles from the initial scaffold; and forming the macroporous tissue engineering scaffold having the embedded controlled release system, wherein the nanoparticles are retained and dispersed in the macroporous tissue engineering scaffold.

In embodiments, the irradiating occurs for about 0.1-100 minutes, 1-10 minutes, or about 5 minutes.

In embodiments, the nanoparticles are homogeneously dispersed and embedded in the macroporous tissue engineering scaffold, and the nanoparticles are attached to and/or embedded in the IPN of the first and second polymer chains.

In embodiments, the macroporous tissue engineering scaffold has macropores and the macropores have an average diameter/pore size of about 30-450 μm.

In embodiments, the polymer-initiator solution is provided in the container in an amount sufficient to fill the container at least about 0.001-1 mm higher than the sugar sphere porogen scaffold template, thereby providing a macroporous tissue engineering scaffold comprising a top layer having a thickness of 0.001-1 mm and a porosity less than 40% v/v that is substantially free of macropores; and a bottom porous layer having a porosity greater than about 60% v/v, or about 70% v/v, or about 80% v/v, or about 90% v/v, and includes macropores having an average diameter/pore size of about 30-450 μm.

The system of the present disclosure can be used to control the release rates, such as long-time release and/or short-time release of the drug substance. For example, the sugar sphere porogen scaffold template can comprise a second type of nanoparticles which are attached to the surface of the sugar spheres and may comprise a fifth polymer and the first drug substance. The fifth polymer can be different from the fourth polymer and thus have different degradation rate from the fourth polymer.

The system of the present disclosure can also be used to control the release rates or two or more different drug substance. For example, the sugar sphere porogen scaffold template may comprise a third type of nanoparticles which are attached to the surface of the sugar spheres and comprise a sixth polymer and a second drug substance. The sixth polymer can be the same or different from the fourth and/or fifth polymers. The second drug substance may be different from the first drug substance.

In embodiments, the first, second and/or the third nanoparticles are homogeneously dispersed in the macroporous tissue engineering scaffold, and may be attached to and/or embedded in the IPN of the first, second or third polymer chains.

In embodiments, the first, second or third nanoparticles may have an average diameter/particle size of 10-1000 nm.

In embodiments, the fifth and/or sixth polymers are selected from the group of poly(lactide-co-glycolide) (PLGA), 50:50 poly(D, L-lactide-co-glycolide) (PLGA), Poly(hydroxyethyl methacrylate) (poly(HEMA)) and copolymers of HEMA, polystyrene (PS), Polyethylene glycol (PEG) and copolymers of PEG, Poly(glycerol adipate) (PGA), Poly(methyl acrylate) (PMA) and copolymers of PMA, poly(acrylic acid) (PAA) and copolymers of PAA, and a combination thereof.

In embodiments, the macroporous tissue engineering scaffold is a periodontal scaffold for a dental implant. In embodiments, the implanting includes fixing the system to the defective dental site by sutures, screws, and/or adhesives, for example by the suture. The crystalline nanostructures may be crystalline nanofibers and at least about 95% of the crystalline nanofibers can be maintained in the system during the implantation of the dental implant. Further, the crystalline nanofibers can be maintained in the macroporous tissue engineering scaffold after at least 50 repeated cycles of heating up to a temperature of about 75° C., deforming and shaping at the temperature up to about 75° C., and cooling.

The macroporous tissue engineering scaffold may have an air-inhibited layer having a porosity of less than about 50% v/v, less than about 40% v/v, less than about 30% v/v, less than about 20% v/v, or less than about 10% v/v and is substantially free of macro-pores; and a porous layer having a porosity higher than about 50% v/v, higher than about 60% v/v, higher than about 70% v/v, higher than about 80% v/v, higher than about 90% v/v, or about 90-99% and comprising macro-pores.

The periodontal membrane can be implanted such that the air-inhibited layer is on top and in contact with the air and the porous layer is on bottom and in contact with the defective dental site. The unique bilayer structure of the macroporous scaffold is advantageous in that the drug substance can be directionally released to the defective dental site, and the air-inhibited layer can substantially prevent tissue growth, and the porous layer can facilitate tissue growth and augment cellular regenerative processes.

In embodiments, the macroporous tissue engineering scaffold having the nanoparticles may release the first drug substance in a first controlled release rate in an oral environment of the subject, the first controlled release rate determined by a degradation rate of the macroporous tissue engineering scaffold and/or a degradation rate of the fourth polymer in the oral environment of the subject.

In embodiments, the macroporous tissue engineering scaffold may further comprise the second type of nanoparticles (comprising a fifth polymer and the first drug substance) dispersed in the macroporous tissue engineering scaffold, and the macroporous tissue engineering scaffold further releases the first drug substance at a second controlled release rate determined by the degradation rate of the macroporous tissue engineering scaffold and a degradation rate of the fifth polymer.

In embodiments, the degradation rates of the fourth and fifth polymers are the same in the oral environment of the subject, and the first and second controlled release rates are the same.

In embodiments, the degradation rates of the fourth and fifth polymers are different in the oral environment of the subject, and the first and second controlled release rates are different.

In embodiments, the macroporous tissue engineering scaffold may further comprise the third type of nanoparticles (comprising a sixth polymer and the second drug substance) dispersed in the macroporous tissue engineering scaffold, and the macroporous tissue engineering scaffold further releases the second drug substance at a third controlled release rate determined by the degradation rate of the macroporous tissue engineering scaffold and a degradation rate of the sixth polymer.

In embodiments, the degradation rates of the fourth, fifth and sixth polymers are all the same, and the first, second and third controlled release rates are the same.

the sixth polymer has a degradation rate different from the degradation rate of the fourth or fifth polymer in the oral environment of the subject,

In embodiments, the degradation rates of the fourth, fifth and sixth polymers are different from each other, and the first, second and third controlled release rates are different.

Another aspect of the present disclosure provides a system having a macroporous tissue engineering scaffold and a controlled release composition embedded in the macroporous scaffold, the system comprising: the macroporous tissue engineering scaffold comprising: a first polymer and a second polymer in a weight ratio of between about 50:50 and about 99:1, the first polymer being a biodegradable linear polymer having first polymer chains, the second polymer being a biodegradable polymer having second polymer chains each of which has two or more functional groups, at least a portion of the second polymer chains being coupled (including chain extended and/or at least partially crosslinked) with each other through the two or more functional groups, wherein the macroporous scaffold has an IPN or SIPN between the first and coupled second polymer chains, wherein the second polymer chains are interspersed throughout the first polymer chains, and wherein the first polymer is at least partially crystallized and at least partially phase separated from the coupled second polymer, the first polymer has a crystallinity of about 10-90% or about 20-80%, the first polymer having crystalline nanostructures homogeneously dispersed in the interpenetrating polymer network (IPN); and the controlled release composition comprising: first nanoparticles comprising a fourth polymer and a first drug substance homogeneously dispersed in the fourth polymer, and wherein the first nanoparticles are dispersed in the macroporous scaffold and the IPN or SIPN.

The details of the compositions, properties, and structures of the system, the macroporous scaffold and the controlled release composition are detailed herein above. The selection of the first, second, third and fourth polymers are also detailed above.

As used herein, the term “an interpenetrating polymer network (IPN)” refers to a type of polymeric or elastomer material comprising two or more chemically distinct polymer networks which coexist and intertwine with each other and are at least partially interlaced on a polymer scale but not covalently bonded to each other, wherein one network interpenetrating through the other polymer network, having a structure that is homogeneous down to the segmental level such as in microscale level, and/or having a structure that has nanoscale phase separation of the two or more chemically distinct networks. One type of IPN can be a network of two chemically crosslinked polymers. Another type of IPN can include a linear or branched polymer which forms crystalline nanostructures such as crystalline nanofibers or crystalline nanotubes in the presence of a network of a crosslinked polymer and the crystalline nanostructures can act as a polymer network to interlock with the crosslinked polymer network. The IPN can thus have a chemically crosslinked polymer network and a crystalline polymer nanostructure network, in which one polymer network interpenetrates the other polymer network, having a structure that is homogeneous down to the segmental level such as in microscale level, and having a structure that has nanoscale phase separation of the crystalline polymer nanostructures and the amorphous crosslinked polymer. The two or more interlocked polymer networks cannot be separated at its intended application temperatures unless chemical bonds are broken. Simply mixing two or more polymers does not create an interpenetrating polymer network (but only forms a polymer blend), nor does creating a polymer network out of more than one kind of monomers which are bonded to each other to form one network (which is just a heteropolymer or copolymer). The nanoscale phase separation may be that one of the polymers can crystallize and form crystalline nanostructures such as crystalline nanofibers or crystalline nanotubes. The IPN can be prepared by a process in which the first and second component networks are formed simultaneously in the presence of each other. For example, the process can be in-situ polymerizing or crosslinking a polymer to form a crosslinked polymer network and simultaneously crystallizing another polymer to form crystalline nanostructures such that the crosslinked polymer network and the crystalline nanostructures are intertwined and interlocked with each other. The IPN can also be prepared sequentially by a process in which the second component network is formed following the formation of the first component network.

As used herein, the term “a semi-interpenetrating polymer network (SIPN)” refers to a type of polymeric or elastomer material comprising one or more polymer network(s) and one or more linear or branched polymer(s), characterized by the penetration on a molecular scale of at least one of the networks by at least some of the linear or branched polymers, having a structure that is homogeneous down to the segmental level such as in microscale level, and/or having a structure that has nanoscale phase separation of one of the polymer networks from the linear or branched polymer chains. The SIPN can be prepared by a process in which the linear or branched polymer are formed simultaneously when the other polymer network is formed. For example, the linear or branched polymer can be formed while the other polymer is crystallized to form the crystalline nanostructures. The SIPN can also be prepared sequentially by a process in which the linear or branched components are formed following the completion of the reactions that lead to the formation of the network(s) or vice versa. The “IPN” or “SIPN” can be present in a biodegradable polymeric material, membrane, and scaffold. As used herein, the terms “interpenetrating polymer network (IPN)” and “semi-interpenetrating polymer network (SIPN)” are used interchangeably.

### EXAMPLES

### Example 1

Preparation of Biodegradable Polymers

1. Poly (L-Lactic Acid) (PLLA)

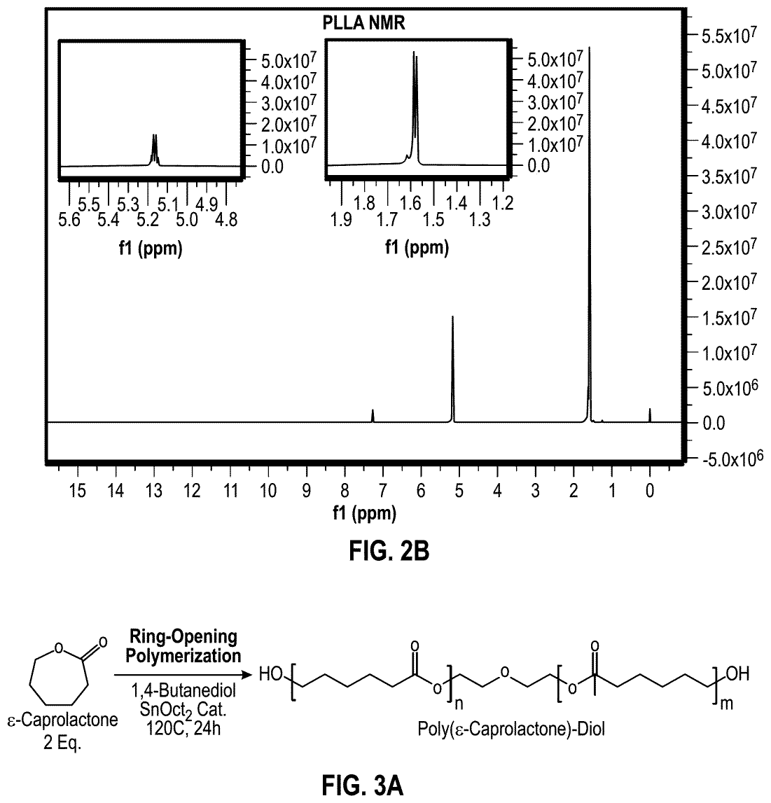

A novel biomaterial is made from crystalline poly (L-lactic acid) (PLLA) and poly (ε-caprolactone) (PCL). PLLA was purchased or synthesized by ring opening metastasis polymerization by a unilateral or bilateral alcohol initiator (such as benzyl alcohol), in bulk, as shown in FIG. 2A. FIG. 2B shows 1H NMR of PLLA polymer in CDCl₃. The 5.15 ppm represents CH shift, and 1.58 ppm represents —CH₃ shift. The tetramethylsilane (TMS) peak is at 0 ppm and the CDCl₃ peak is at 7.4 ppm. The molecular weight of PLLA is 150,000 (Resomer Res L207S) kDa, as purchased from Evonik. As used herein, and unless specified otherwise, the molecular weights of the polymers are provided as weight average molecular weights.

2. Synthesis of Poly (ε-Caprolactone) (PCL)

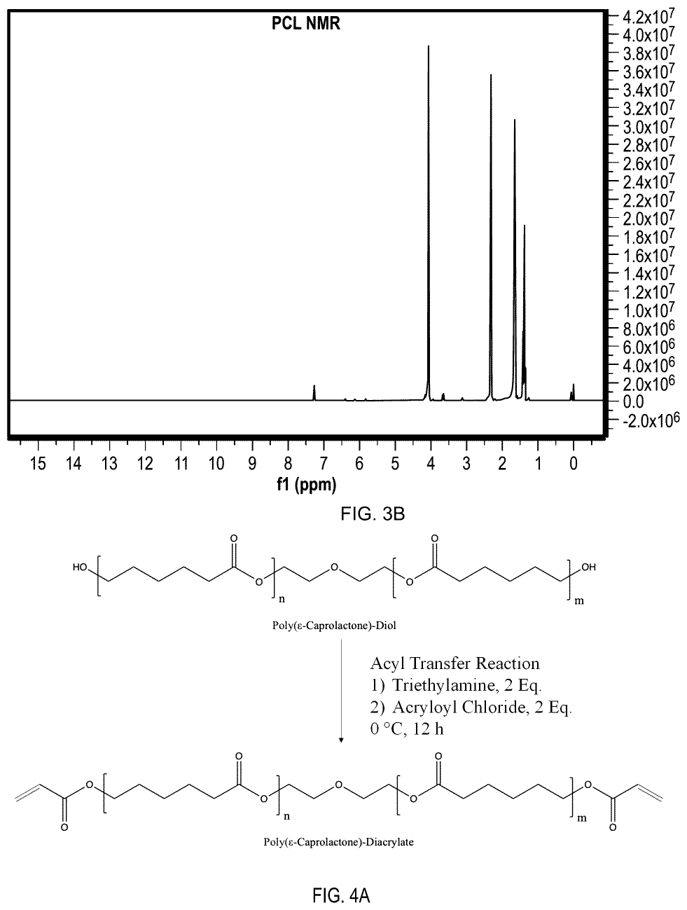

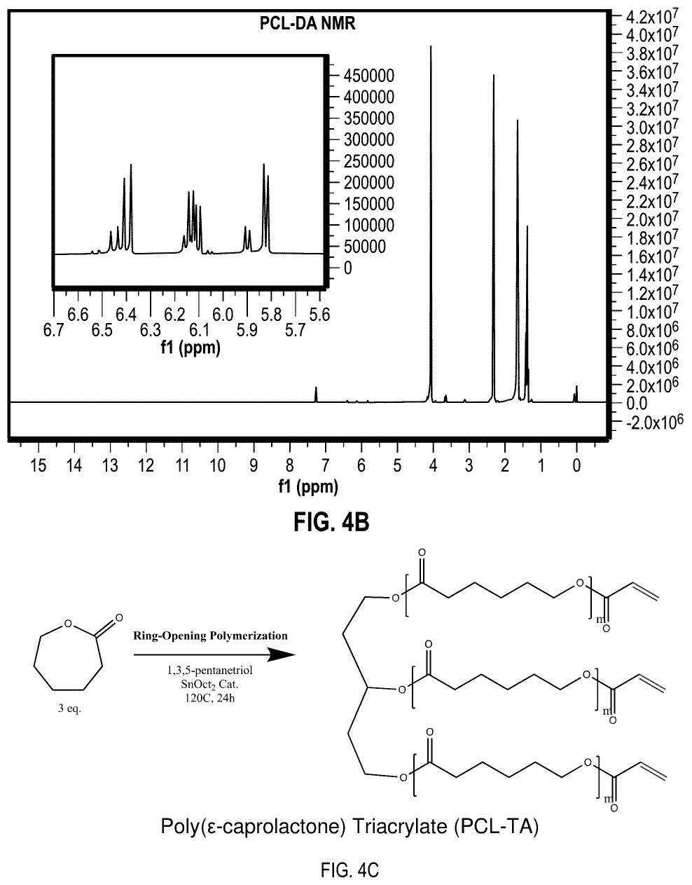

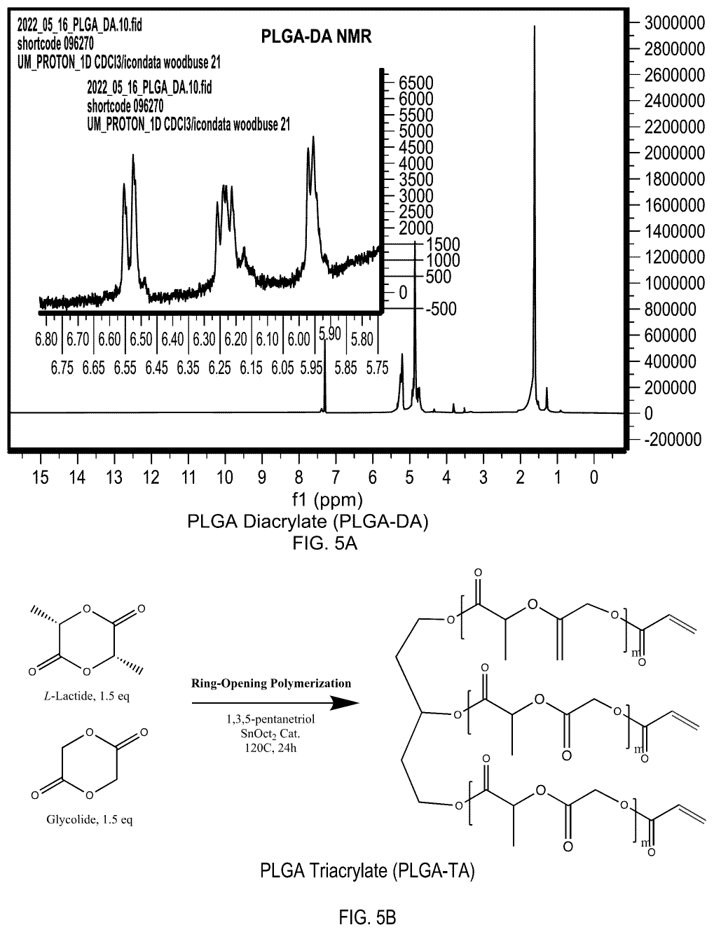

PCL was synthesized by ring opening metastasis polymerization at molecular weight between 1 kDa to 20 kDa with a bifunctional initiator (such as, 1,4-butanediol in this example) as shown in FIGS. 4 and 5. In the preparation of poly-ε-caprolactone having molecular weight (MW) of 10,471 Da as determined by gel permeation chromatography (GPC), 10 mL of ε-caprolactone monomer was added into a 50 mL round-bottom flask along with 89 uL of 1,4-butanediol. 6.5 uL of Tin (II) 2-ethylhexanoate catalyst was added directly to the round-bottom flask. The resulting solution was purged from oxygen and stirred at a low speed under a vacuum, the reaction proceeded at 120° C. for 2 hours, as shown in FIG. 3A. The resulting product was a viscous white liquid. The round-bottom flask was allowed to cool to room temperature such that a white solid formed. Dichloromethane was added to dissolve the white solid. Once dissolved, the resulting solution was poured into 5× excess mL of methanol at 0° C. in a 1000 mL beaker to precipitate the white poly-ε-caprolactone diol solid. This solution was then centrifuged to concentrate the poly-ε-caprolactone diol solid such that the supernatant could be removed. This process was repeated three times to remove unreacted monomer. The solid poly-ε-caprolactone diol was allowed to dry for 2 days in a vacuum chamber before further use. FIG. 3B shows 1H NMR spectrum of PCL in CDCl₃.

3. Synthesis of Poly (ε-Caprolactone) Diacrylate (PCL-DA)

PCL was bifunctionally modified at its terminus from terminal hydroxyl functional groups to acrylic functional groups through nucleophilic substitution reaction with acryloyl chloride, yielding PCL-DA, as shown in FIG. 4A and FIG. 4B. To synthesize poly-ε-caprolactone diacrylate, 5 grams of poly-ε-caprolactone diol was transferred into a 50 mL round-bottom flask in an ice bath and was solubilized in dichloromethane to form a solution. 135 uL of triethylamine was added to the resulting solution stirring at moderate speed of 10-5000 rpm to activate the terminal hydroxyl groups of PCL. The round-bottom flask was sealed, and the reaction was allowed to proceed for 15 minutes before 78 uL of acryloyl chloride was added dropwise into the solution at 0° C. The reaction was allowed to proceed overnight under moderate stirring, slowly warming to room temperature in the melting-ice bath. After this time had elapsed, the reaction was precipitated into five times volume excess methanol at 0° C. in a 1000 mL beaker to precipitate the white poly-ε-caprolactone diacrylate (PCL-DA) solid; repeated 3 times to remove unreacted acryloyl chloride. This solution was centrifuged to concentrate the poly-ε-caprolactone diacrylate solid such that the supernatant could be removed. The solid poly-ε-caprolactone diacrylate was allowed to dry for 2 days in a vacuum chamber before it is ready for biomaterial applications.

The ring-opening metathesis polymerization (ROMP) occurred in bulk through a melt polymerization. The structure and purity were assessed by nuclear magnetic resonance spectroscopy (as shown in FIG. 3B and FIG. 4B) and Fourier transformed infrared spectroscopy; molecular weight was assessed by gel permeation chromatography (GPC). The end group modification was confirmed by nuclear magnetic resonance spectroscopy, as shown in FIG. 4B.

4. Synthesis of Poly (ε-Caprolactone) Triacrylate (PCL-TA)

Poly (ε-caprolactone) triol was trifunctionally modified at each of the three terminus from terminal hydroxyl functional groups to acrylic functional groups through nucleophilic substitution reaction with acryloyl chloride, yielding poly (ε-caprolactone) triacrylate (PCL-TA), as shown in FIG. 4C. The trifunctional PCL-TA was prepared in the same way as PCL-DA (triethylamine activation, acryloyl chloride at 0° C. for 12 h) discussed above.

Specifically, to synthesize poly-ε-caprolactone triacrylate (PCL-TA), 5 grams of poly-ε-caprolactone (PCL) triol was transferred into a 50 mL round-bottom flask in an ice bath and was solubilized in dichloromethane. 135 uL of triethylamine was added to the resulting solution which was stirred at moderate speed of 10-5000 rpm to activate the terminal hydroxyl groups of PCL triol. The round-bottom flask was sealed, and the reaction was allowed to proceed for 15 minutes before 78 uL of acryloyl chloride was added dropwise into the solution at 0° C. The reaction was allowed to proceed overnight under moderate stirring, slowly warming to room temperature in the melting-ice bath. After this time had elapsed, the reaction was precipitated into five times volume excess methanol at 0° C. in a 1000 mL beaker to precipitate the white poly-ε-caprolactone triacrylate (PCL-TA) solid; repeated 3 times to remove unreacted acryloyl chloride. This solution was centrifuged to concentrate the poly-ε-caprolactone triacrylate solid such that the supernatant could be removed. The solid poly-ε-caprolactone triacrylate was allowed to dry for 2 days in a vacuum chamber before it was ready for biomaterial applications.

4. Synthesis of Poly(Lactic-Co-Glycolic Acid) Diacrylate (PLGA-DA)

The poly(lactic-co-glycolic acid) (PLGA) was bifunctionally modified at its terminus from terminal hydroxyl functional groups to acrylic functional groups through nucleophilic substitution reaction with acryloyl chloride, yielding PLGA-DA. The 1H NMR analysis of the diacrylation reaction to form the PLGA-DA is shown in FIG. 5A, as observed in CDCl₃. The difunctional PLGA-DA was prepared in the same way as PCL-DA (triethylamine activation, acryloyl chloride at 0° C. for 12 h) discussed above. The purification process and characterization method of the PLGA-DA polymer were also the same as those of PCL-DA discussed above. 5 grams of poly(lactic-co-glycolic acid) diol was transferred into a 50 mL round-bottom flask in an ice bath and was solubilized in dichloromethane to form a solution. 1.1 molar equivalent of chain end triethylamine (TEA) catalyst was added to the resulting solution stirring at moderate speed of 10-5000 rpm to activate the terminal hydroxyl groups of poly(lactic-co-glycolic acid) diol. The round-bottom flask was sealed, and the reaction was allowed to proceed for 15 minutes to form a solution. 1.5 molar equivalent of acryloyl chloride was added dropwise into the solution at 0° C. The reaction was allowed to proceed overnight under moderate stirring, slowly warming to room temperature in the melting-ice bath. After this time had elapsed, the reaction was precipitated into five times volume excess methanol at 0° C. in a 1000 mL beaker to precipitate the white poly(lactic-co-glycolic acid) diacrylate (PLGA-DA) solid; and repeated 3 times to remove unreacted acryloyl chloride. This solution is centrifuged to concentrate the PLGA-DA solid such that the supernatant could be removed. The solid PLGA-DA was allowed to dry for 2 days in a vacuum chamber before it was ready for biomaterial applications. In this study, the molar ratios of the PLGA diol to triethylamine (TEA) to acryloyl chloride is about 1:2.2:3.

The structure and purity of the PLGA-DA were assessed by nuclear magnetic resonance spectroscopy (as shown in FIG. 5A) and Fourier transformed infrared spectroscopy; molecular weight is assessed by gel permeation chromatography (GPC). End group modification was confirmed by nuclear magnetic resonance spectroscopy, as shown in FIG. 5A.

5. Synthesis of Poly(Lactic-Co-Glycolic Acid) Triacrylate (PLGA-TA)

The poly(lactic-co-glycolic acid) (PLGA) triol was trifunctionally modified at its three terminus from terminal hydroxyl functional groups to acrylic functional groups through nucleophilic substitution reaction with acryloyl chloride, yielding PLGA-TA, as shown in FIG. 5B. The trifunctional PLGA-TA was prepared in the same way as PLGA-DA (triethylamine activation, acryloyl chloride at 0° C. for 12 h) discussed above. The purification process and characterization method of the PLGA-TA polymer were also the same as those of PLGA-DA discussed above. 5 grams of poly(lactic-co-glycolic acid) diol was transferred into a 50 mL round-bottom flask in an ice bath and was solubilized in dichloromethane. 1.1 molar equivalent of chain end triethylamine (TEA) catalyst was added to the resulting solution stirring at moderate speed of 10-5000 rpm to activate the terminal hydroxyl groups of poly(lactic-co-glycolic acid) triol. The round-bottom flask was sealed, and the reaction was allowed to proceed for 15 minutes to form a solution. 1.5 molar equivalent of acryloyl chloride was added dropwise into the solution at 0° C. The reaction was allowed to proceed overnight under moderate stirring, slowly warming to room temperature in the melting-ice bath. After this time had elapsed, the reaction was precipitated into five times volume excess methanol at 0° C. in a 1000 mL beaker to precipitate the poly(lactic-co-glycolic acid) triacrylate (PLGA-TA) polymer solid; and repeated 3 times to remove unreacted acryloyl chloride. This solution was centrifuged to concentrate the PLGA-TA solid such that the supernatant could be removed. The solid PLGA-TA was allowed to dry for 2 days in a vacuum chamber before it was ready for biomaterial applications. Any “triol” initiator can be used for the synthesis of PLGA triol, such as 1,3,5-pentanetriol initiator, glycerol initiator, some PEG-ylated multifunctional triol initiator. In this study, 1,3,5-pentanetriol initiator was used. In this study, the molar ratios of the PLGA triol to triethylamine (TEA) to acryloyl chloride is about 1:3.3:4.5.

### Example 2

Synthesis of Interpenetrating Polymer Network (IPN) or Semi-Interpenetrating Polymer Network (SIPN) of PLLA/PCL

In one study, two biodegradable polymeric material samples were prepared having IPN or SIPN of PLLA and PCL in weight ratios of 60:40 and 80:20, respectively. The PLLA and PCL-DA in the designed weight ratios (60:40 and 80:20 respectively) were combined and fully dissolved in tetrahydrofuran (THF) to form a 10 w/v polymer solution. The polymer solution was injected into a separate Irgacure stock solution in a concentration of 3 mM via a pipette measuring 3.33% v/v. This resulting polymer-Irgacure solution was rapidly stirred and mixed thoroughly and then exposed to high intensity UV lights to form an IPN or SIPN by the in-situ polymerizing or coupling of the PCL-DA polymer chains within a PLLA component, where the majority component is PLLA, using Irgacure 2959 (excitation wavelength: 365 nm) as a photo initiator. The in-situ polymerizing/coupling included but was not limited to chain-extending of the PCL-DA. Other suitable radical initiators can also be used for in-situ UV and/or thermal crosslinking of PCL-DA, such as 1-vinyl-2 pyrrolidinone (NVP, excitation wavelength: 365 nm) and lithium phenyl-2,4,6-trimethylbenzoylphosphinate (LAP, excitation wavelength: 405 nm).

Another two biodegradable polymer material samples were prepare in the same method as the first two samples prepared above in this example, except that small amount of PCL-TA was used to replace the PCL-DA. The weight ratio of the PCL-TA to PCL-DA was about 0.075:1. The rest of the preparation process was the same as detailed above in this Example. The PCL-TA was used to crosslink at least a portion of the PCT-TA polymer chains with each other and/or with the PCL-DA polymer chains in addition to the chain extension of the PCL-DA and/or PCL-TA polymer chains.

The amount of the PCL-TA can be tuned to adjust the crosslinking density of the PCL-TA and PCL-DA polymer chains in the biodegradable polymeric materials based on specific applications. The weight ratios of PCL-TA to PCL-DA can be in a range of about 0.001:1 to about 1:1, or about 0.01:1 to about 0.5:1, or about 0.01:1 to about 0.2:1, or about 0.01:1 to about 0.1:1.

The chain extending and/or the partial crosslinking of the PCL-TA and/or PCL-DA polymers within a PLLA mesh creates a memorized microstructure (MMS), where the microstructure is the direct result of the specific fabrication protocol. In this way, the linear PLLA forms the bulk of the resulting material, which is weaved and captured within a chain extended and/or crosslinked PCL mesh from the radical-induced chain extending and/or crosslinking of PCL-DA and PCL-TA. The biodegradable polymeric materials prepared by the process of the present disclosure advantageously have the memorized microstructure (MMS) with the interpenetrating polymer network (IPN) or semi-interpenetrating polymer network (SIPN) of two distinct polymers. In the resulting biodegradable polymeric material, the PLLA polymer imparts mechanical rigidity to the material and its crystallinity lends itself to crystalline nanofiber formation by thermally induced phase separation. The PCL polymer imparts thermos-sensitivity due to its relatively lower melting temperature and high elastic deformation. Both PLLA and PCL polymers are regarded as nontoxic, biodegradable, biocompatible polyesters which are FDA approved for human use.

In this example, a biodegradable polymeric material having the IPN or SIPN microstructure was prepared with a first biodegradable linear polymer PLLA and a second biodegradable polymer PCL-DA, and optionally a small amount of a third polymer of PCL-TA. Other suitable biodegradable polymer systems can also be used to prepare the biodegradable material having the IPN or SIPN microstructures. The physical properties, biodegradation rates, microstructures, morphologies, and clinical handling properties of the biodegradable polymeric materials can be adjusted based on the specific applications.

For example, the first polymer, PLLA, may be fabricated as PLLA diacrylate capable of chain extension. In some examples, the PLLA may also be replaced by poly-3-hydroxybutyrate (P3HB), PLGA, or a copolymer containing PLLA, each of which is capable of crystallizing and forming nanofibers due to its bulk crystallinity. In some examples, the second polymer, polycaprolactone (PCL), may be replaced by poly-4-hydroxybutyrate (P4HB) which has similar thermal properties but with increased hydrophilicity. In some other examples, PCL and PHB polymers may be blended as either the crosslinked network or interpenetrating polymer. The molecular weight of the first polymer may be between 500 Da and 200 kDa. The second polymer has an initial molecular weight between 500 Da and 200 KDa before the in-situ reaction, and a final molecular weight between 20 KDa and 500 KDa after the in-situ reaction. The final molecular weight is greater than the initial molecular weight for the second polymer.

For example, the biodegradable polymeric material having IPN or SIPN of PLLA/PCL may be suitable for long term drug delivery applications. This is because both the PLLA and PCL polymers have long degradation time. PLLA has an approximate degradation time of over 24 months. The PCL polymer has a degradation time of approximately two to three years. The biodegradable polymeric material may require different degradation rates for different applications. The degradation rate of the biodegradable polymeric material can be altered by adding other suitable biodegradable polymers or copolymers to the PLLA/PCL system or replacing one or both of the PLLA and PCL polymers. One suitable biodegradable polymer is PLGA which has a tailorable degradation time from about a few weeks to several months by varying the poly(lactic acid) to poly(glycolic acid). The PLGA polymer can be used to replace part or all of the PCL polymer as detailed in Examples 3 and 4 below.

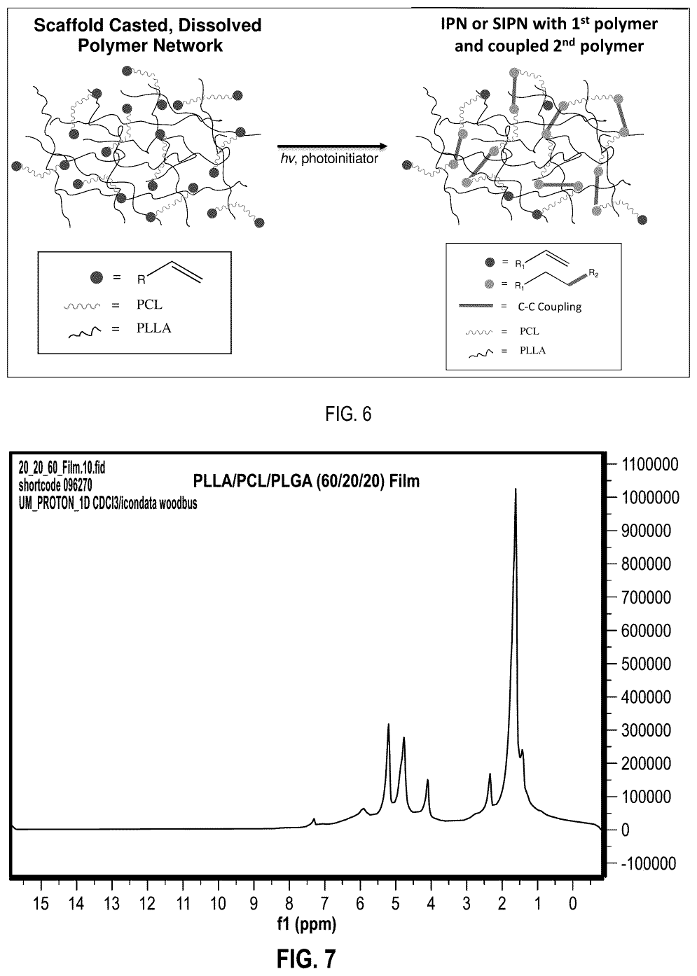

FIG. 6 schematically demonstrates the interpenetrating of the first and second polymer networks, and the coupling of the second polymer chains, using PLLA as the first polymer and PCL-DA as the second polymer, as an example.

### Example 3

Synthesis of Interpenetrating Polymer Networks (IPN) or Semi-Interpenetrating Polymer Networks (SIPN) of PLLA/PLGA

The biodegradable polymeric material samples having the IPN or SIPN of PLLA/PLGA were prepared in the similar method as the biodegradable polymeric material samples having the IPN or SIPN of PLLA/PCL discussed above in Example 2.

Two biodegradable polymeric material samples were prepared having IPN or SIPN of PLLA and PLGA in weight ratios of 60:40 and 80:20, respectively. The PLLA and PLGA-DA (prepared in Example 1) in the pre-designed weight ratios were dissolved and mixed in tetrahydrofuran (THF) to form a 10 w/v polymer solution. The polymer solution was injected into a separate Irgacure solution to form a polymer-Irgacure solution. The polymer-Irgacure solution was exposed to high intensity UV lights to form an IPN or SIPN by the in-situ polymerizing/coupling of the PLGA-DA polymer chains within a PLLA component, where the majority component was PLLA, using Irgacure 2959 (excitation wavelength: 365 nm) as a photo initiator. Similarly, other suitable radical initiators can also be used for UV or thermal polymerization/coupling of the PLGA-DA polymer, such as 1-vinyl-2 pyrrolidinone (NVP, excitation wavelength: 365 nm) and lithium phenyl-2,4,6-trimethylbenzoylphosphinate (LAP, excitation wavelength: 405 nm). The in-situ polymerizing or coupling included but was not limited to chain-extending the PLGA-DA polymer chains with each other.

Another two biodegradable polymeric material samples were prepared having PLLA and PLGA in a weight ratio of 60:40 and 80:20, respectively in the same process as that of the first two samples prepared above in this example, except that a small amount of PLGA-TA was used to replace the PLGA-DA. In these two samples, about 7.5 wt. % of the PLGA-DA was replaced with PLGA-TA to at least partially crosslink the PLGA-TA and/or PLGA-DA polymer chains. The PLLA, PLGA-DA and PLGA-TA in the predesigned weight ratios (60:37:3 and 80:18.5:1.5, respectively) were dissolved and mixed in tetrahydrofuran (THF) to form a 10 w/v polymer solution. The rest of the synthesis steps were the same as those of the first two samples prepared in this example. The in-situ polymerizing/coupling included but was not limited to chain-extending and at least partially crosslinking of the PLGA-DA and PLGA-TA polymer chains.

The amount of the PLGA-TA can be tuned to adjust the crosslinking density of the PLGA-TA and PLGA-DA polymer chains in the biodegradable polymeric materials based on specific applications. The weight ratios of PLGA-TA to PLGA-DA can be in a range of about 0.001:1 to about 1:1, or about 0.01:1 to about 0.5:1, or about 0.01:1 to about 0.2:1, or about 0.01:1 to about 0.1:1.

### Example 4

Synthesis of Interpenetrating Polymer Networks (IPN) or Semi-Interpenetrating Polymer Networks (SIPN) of PLLA/PCL/PLGA

In this study, biodegradable polymeric material samples having IPN and SIPN of three distinct polymers were synthesized using the same method discussed in Examples 2 and 3, except that there were three distinct polymers in the IPN or SIPN.

Three biodegradable polymeric material samples were prepared from PLGA-DA, PCL-DA and PLLA (three polymers system) in weight ratios of 30:10:60, 20:20:60 and 10:30:60 to form the IPNs (or SIPNs) of PLGA, PCL and PLLA polymers chains the designed weight ratios in the same method discussed above in this Examples 2 and 3. The PLGA-DA, PCL-DA and PLLA in the pre-designed weight ratios were dissolved and mixed in tetrahydrofuran (THF) to form a 10 w/v polymer solution. The rest of the preparation steps were the same as discussed above in Examples 2 and 3. The 1H NMR analysis of the PLLA/PCL/PLGA in a weight ratio of 60:20:20 is shown in FIG. 7, as observed in CDCl₃.

The physical properties of the biodegradable polymeric materials were characterized and detailed below.

The purpose of introducing PLGA-DA or PLGA-TA to the TS-MMS membrane or scaffold is to modulate the degradation time and kinetics, while maintaining the thermosensitive properties of the membrane or scaffold.

### Example 5

Preparation of Surgical Membranes

The methods of fabricating two tissue engineering constructs, such as surgical membranes and macroporous scaffolds, are detailed below. Two key features are the direct result of the novel fabrication protocol of the disclosure. First, nanofibers result from a nanoscale thermally induced phase separation of PLLA through a spinodal decomposition pathway. Nanofibers are well-described design motifs to improve protein adsorption, and facilitate cell adhesion within a synthetic matrix. Secondly, the materials feature a reversible thermosensitive memorized microstructure which enables the efficient delivery of nanofibers and embedded technologies to any shaped defect site, while preserving their advantageous features.

In the study, the thermosensitive memorized microstructure (TS-MMS) material of the present disclosure was fabricated as a surgical membrane which can be used, for example, for periodontal tissue engineering. Hollowed rectangular casting mold outlines can be 3D printed from PLLA. The dimensions of the molds are not particularly limited and can vary depending on the needs of the end use. In this Example, these molds had the dimensions of: 60×10×1 mm, as shown in FIG. 8. The 3D printed mold outlines were attached to the positively charged side of glass microscope slide using Gorilla Glue Super Glue® (as an example) and allowed to dry in a chemical fume hood for two days prior to polymer casting.

A 10% w/v polymer solution in tetrahydrofuran (THF) is prepared from 40% wt/wt poly-ε-caprolactone diacrylate (PCL-DA) (MW=10.4 kDa) and 60% wt/wt poly-L-lactide (PLLA) (MW=150 kDa). Examples of suitable solvents can include tetrahydrofuran (THF), benzene, toluene, dioxane, methanol, ethanol, or mixtures thereof. The solvent selection depends on the solvent's ability to induce thermally induced phase separation for the nanofibers. If nanofibers are not desirable, but rather smooth scaffold walls are desirable, then additional solvents, such as ethyl acetate and methylene chloride, would also be suitable. The polymer solution was then heated, for example, in a convection oven at 62° C. for at least 1 hour until the polymer solution was fully dissolved. A separate stock solution of photo initiator was prepared by 1000-fold w/v dilution of the Irgacure® 2959 in methanol to obtain a final concentration of 3 mM. Ready polymer molds, described above, were transferred to a FisherScientific® UV Crosslinking Chamber (λ=356 nm) powered with E˜10 J. Dry ice in an insulated container was placed near the UV crosslinking chamber alongside the Irgacure stock solution. Prior to removing the dissolved polymer solution, ethanol was poured into a glass beaker to a height of approximately 75 mm, covered with aluminum foil, and placed into a −80° C. freezer.

After the steps above were completed and the polymer solution was completely dissolved, the polymer solution was removed from the convection oven and quickly transferred into a thermally insulated container. The container was rapidly transported to a UV crosslinking chamber where the polymer solution was injected with the Irgacure stock solution via a pipette measuring 3.33% v/v. This resulting polymer-Irgacure solution was rapidly stirred before aliquots of the polymer-Irgacure solution were transfer-pipetted into the film molds sitting in the paused UV crosslinking chamber. Film molds were filled to ˜1.25 mm height with the polymer-Irgacure solution. Once the molds were cast with polymer, the UV crosslinking chamber was shut and exposed to high intensity UV, and the films were allowed to crosslink for 5 min. After the time has elapsed, the film molds were removed from the UV crosslinking chamber and rapidly transferred onto flat surfaces of dry ice. They were allowed to cool for 10 min on the dry ice before being transferred into −80° C. ethanol, where the film molds containing the polymer films were chilled at −80° C. in ethanol for an additional 48 hours to induce nanofiber formation by thermally induced phase separation.

The film molds were removed from the −80° C. ethanol beaker and soaked in a distilled water ice bath that was allowed to warm to room temperature over the course of 2 hours. Once at room temperature, the film molds were removed from the water bath and the solidified polymer films were cut from the molds with a razor blade. The polymer films were dried and lyophilized. Films were assessed for morphology by scanning electron microscopy and stored at −80° C. until use. Prior to use, sterilization is performed by ethylene oxide gas.

### Example 6

Preparation of Macroporous Scaffolds

In the second, the TS-MMS of the present disclosure is fabricated as macro-porous scaffolds from a sugar sphere porogen scaffold template by a modification of a method previously described by Swanson et al. and Wei at al. ((W. B. Swanson, Macropore Design of Tissue Engineering Scaffolds Regulates Mesenchymal Stem Cell Differentiation Fate, Biomaterials (2021); and G. B. Wei., Macroporous and nanofibrous polymer scaffolds and polymer/bone-like apatite composite scaffolds generated by sugar spheres, J. Biomed. Mater. Res. A 78A(2) (2006) 306-315.). D-fructose sugar was melted and emulsified in hot mineral oil containing Span80 surfactant, with a magnetic stir bar. The fructose-mineral oil emulsion was cooled rapidly in an ice bath and solid sugar spheres were washed with hexane to remove residual oil and surfactant. The heterogeneous mixture of sugar spheres was purified by size using molecular sieves (Newark Wire Cloth Co) to select for sugar spheres in desired size ranges (Small: 60-125 μm, Medium: 125-250 μm, and Large: 250-425 μm).

The sugar in the sugar template can be manufactured to be sugar particles in any suitable shapes including but not limited to spheres, cubes, cylinders, cones, rectangular prisms, triangular prisms, or a combination thereof.

In this study, the sugar particles were prepared in the shape of spheres. The sugar spheres can be sugar microspheres having an average particle size or diameter of about 1-1000 μm, about 10-500 μm, about 30-450 μm, or about 60-425 μm.

Sugar spheres of desired size range were loaded into a Teflon mold, in hexane, and annealed at 37° C. to cause partial adhesion to neighboring spheres (Small: 7 minutes, Medium: 9 minutes, Large: 12 minutes). Hexane was removed and the sugar template is dried under vacuum at room temperature. A 10% w/v polymer solution in tetrahydrofuran was prepared from 40% wt/wt poly-ε-caprolactone (MW=10.4 kDa) and 60% wt/wt poly-L-lactide (MW=150 kDa). The polymer solution is then placed in a convection oven at 62° C. for at least 1 hour until the polymer solution was fully dissolved. A separate stock solution of photo initiator was prepared by 1000-fold w/v dilution of the Irgacure® 2959 in methanol to obtain a final concentration of 3 mM. Ready polymer molds, described above, were transferred to a FisherScientific® UV Crosslinking Chamber (λ=356 nm) powered with E˜10 J. Dry ice in an insulated container is obtained and placed near the UV crosslinking chamber alongside the Irgacure stock solution.

After the steps above were completed and the polymer solution was completely dissolved, is the polymer solution was removed from the convection oven and quickly transferred into a thermally insulated container. The container was rapidly transported to the UV crosslinking chamber where the polymer solution was injected with the Irgacure stock solution via a pipette measuring 3.33% v/v. This resulting polymer-Irgacure solution was rapidly stirred before aliquots of the polymer-Irgacure solution were transfer-pipetted into the sugar templates sitting in the paused UV crosslinking chamber. Once the templates are cast with polymer, the UV crosslinking chamber was shut and exposed to high intensity UV, and the films were allowed to crosslink for 5 min. After the time has elapsed, the film molds were removed from the UV crosslinking chamber and rapidly transferred onto flat surfaces of dry ice and immediately stored at −80° C. to induce thermally induced phase separation (TIPS) for 48 hours. After this time, the Teflon vials were submerged in hexane for 24 hours, then distilled water for 24 hours to leach the sugar spheres. Resulting scaffolds were cut with a biopsy punch and stored at −80° C. FIG. 9 shows a photo of an example of a resulting TS-MMS scaffold in a cylinder shape after processing from the sugar sphere porogen method and leaching sugar spheres with water.

### Example 7

Characterizing Crystalline Domain and PLLA Nanofiber Formation

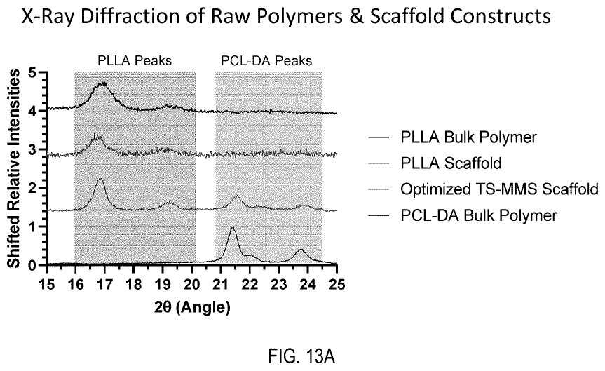

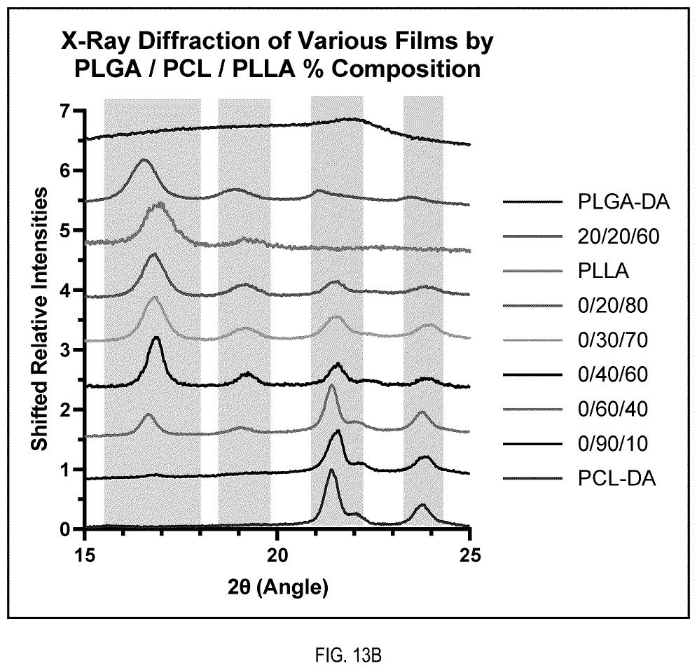

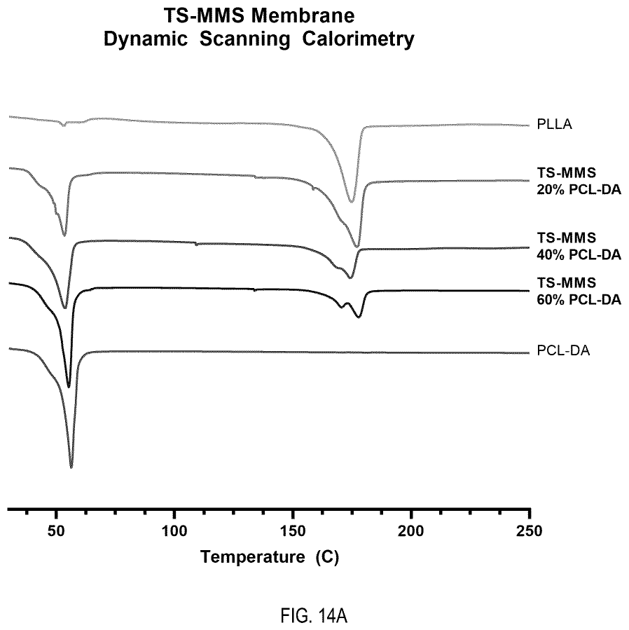

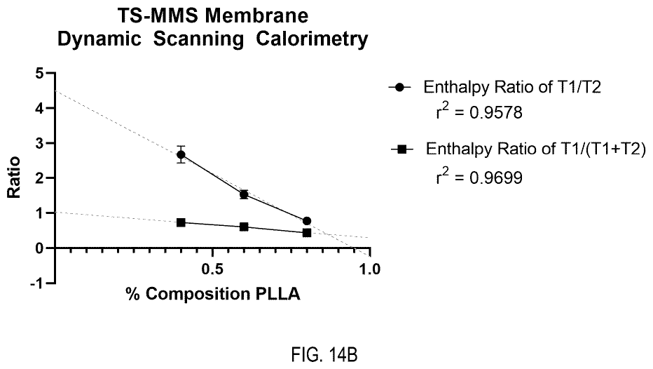

X-ray diffraction was used to measure the crystallinity of the raw polymers and the biodegradable polymeric materials prepared in Examples 2-5 above and to record a molecular signature before and after synthesis processing. The X-Ray diffraction patterns for the raw PLLA polymer, PLLA (100 wt. %) scaffold, PLLA/PCL scaffold, and PCL-DA raw polymer are shown in FIG. 13. The X-Ray diffraction patterns for the raw PLGA, PLLA and PCL-DA polymers, and the membranes having PLGA/PCL/PLLA in different weight ratios are shown in FIG. 14.

As shown in FIGS. 13 and 14, the crystallinity of PLLA was maintained in TS-MMS materials, and was distinct from the signature of PCL-DA or PLGA-DA. The crystallinity of PLLA was maintained from its bulk form and in the scaffold form and also the membranes, indicating no effect of the polymer processing on the crystallinity of the PLLA. The crystallinity of PLLA is critical (more than 50 wt. %, and preferably at least 60 wt. % of the total polymeric material must be the PLLA crystalline polymer) to facilitate the PLLA nanofiber formation by thermally induced phase separation.

The formation of PLLA nanofibers can be resulted from thermally induced phase separation of crystalline PLLA, by its spinodal decomposition from tetrahydrofuran (THF) solvent. The PLLA nanofibers are an advantageous design motif because they facilitate protein adsorption and cell adhesion to the construct, critical for regeneration and tissue integration. The experimental tests were conducted to determine the minimum wt/wt % of PLLA by weight of the total polymers necessary in the TS-MMS compositions (made from PLLA and PCL-DA; and PLLA and PLGA-DA, respectively) of the present disclosure necessary to maintain the nanofibrous architecture of PLLA.

Scanning electron micrographs (SEM) are used to visualize the PLLA nanofibers resulting from thermally induced phase separation (TIPS) as a function of PLLA/PCL-DA weight ratio, and the SEM images are shown in FIGS. 10A-10J. The SEM was also used to observe nanofiber formation as a function of polymer material concentration as cast from the polymer solution, and the SEM images are shown in FIGS. 11A-11D. The SEM was also used to observe the effect of PCL-DA molecular weight on PLLA nanofiber formation, and the SEM images are shown in FIGS. 12A-12C.

The TS-MMS membranes were fabricated from PLLA and PCL-DA polymer in THF solution, as described at 10% wt/v total polymer material. Their morphology was examined by scanning electron microscopy as shown in FIGS. 10A-10J. As shown in FIGS. 10A-10J, it was determined that a minimum amount of about 50 wt. %, preferably 60 wt. % of PLLA by weight of the total polymers is necessary to maintain the PLLA nanofibers. Less than 60% but greater than 40% wt/wt of PLLA resulted in a platelet morphology, as shown in FIG. 10F. Less than or equal to 40% wt/wt PLLA resulted in an amorphous surface character, as shown in FIGS. 10G-10J. This amorphous character might be attributed to phase separation below a critical threshold of PLLA determined to be equal to or greater than 60% wt/wt. The total concentration (% wt/v) of polymer material in the THF polymer solution cast at any given combination, for example 60% PLLA/40% PCL-DA, directly correlated to PLLA nanofiber density and spacing, between 2.5-20% wt/v, as shown in FIGS. 11A-11D, respectively.

The PLLA nanofiber formation did not depend on PCL-DA molecular weight and was rather a function of PLLA, as shown in FIGS. 12A-12C.

### Example 8

Temperature Sensitive Properties of TS-MMS

Dynamic scanning calorimetry (DSC) was used to characterize the temperature sensitive properties of the resulting TS-MMS, and the DSC thermogram curves are shown in FIGS. 13A and 13B. As shown in FIGS. 13A and 13B, the 100% wt. % PLLA control (pure PLLA) exhibited one single melting temperature above 160° C. (at about 170° C.), and the 100 wt. % of PCL-DA (pure PCL-DA) exhibited one single melting temperature of about 50-55° C. The 20% PCL-DA/80% PLLA, 40% PCL-DA/60% PLLA, and 60% PCL-DA/40% PLLA TS-MMS all exhibited two distinct melting temperatures at about 50-55° C. and about 170° C., respectively. Neither sample absorbed significant heat flow or underwent a phase transition at or near physiologic temperature of about 37° C. and 42° C. The DSC test results clearly demonstrate that, in the biodegradable polymeric membranes or scaffolds having IPN or SIPN of the PLLA and PCL-DA polymers, the PCL or PCL-DA component of the TS-MMS system of the disclosure is directly responsible for its temperature sensitive behavior. The PCL in the TS-MMS system acts as a molecular switch which was cast around the PLLA nanofibers and served to deliver the PLLA nanofibers in a unique geometry.

The ideal range for a TS-MMS material to be used in dental implant applications is 45-55° C. melting temperature. Beyond 100° C., the melting temperature is too high to be relevant to the applications described. The TS-MMS materials show two melting temperature peaks at 50-55° C. indicating its shape memory potential, and a second peak at 160-175° C. which is indicative of the high melting temperature of the PLLA polymer. A TS-MMS having a melting temperature lower than about 40° C., or lower than about 42° C. is irrelevant as the physiologic temperature of a subject would be above the melting temperature of the TS-MMS material, therefore the TS-MMS material loses its rigidity.

The enthalpy changes during the DSC test at 52° C. are directly proportional to the weight ratio of the PCL-DA, as shown in FIG. 14.

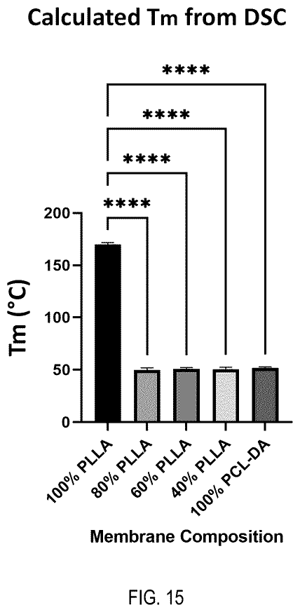

No difference in the calculated melting temperature (Tm) was noted as a function of composition of PLLA/PCL-DA, however TS-MMS have a significantly lower melting temperatures than the pure PLLA membranes, as shown in FIG. 15.

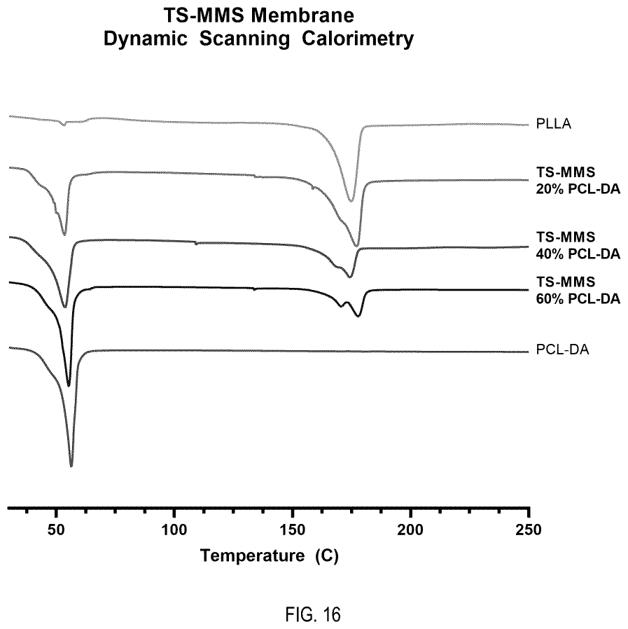

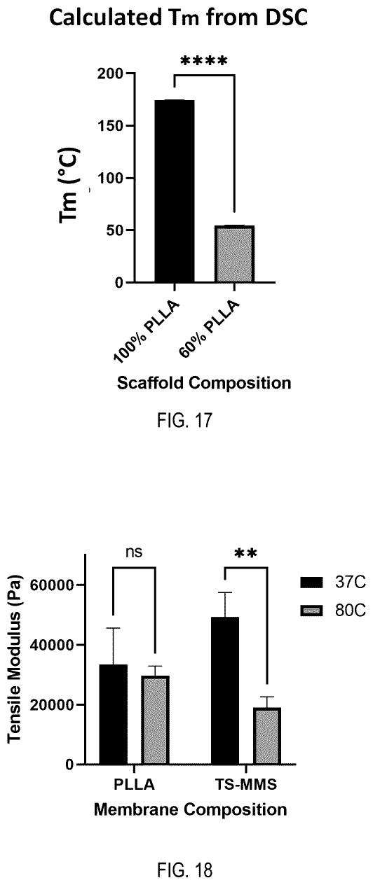

The same phenomena were also observed in macroporous scaffolds, which validated the hypothesis that TS-MMS thermal properties and thermosensitive behavior are only a function of compositions, especially the types of polymers, not the fabrication method, as shown in FIG. 16 and FIG. 17.

As a proof of concept for the memorized microstructure behavior, membranes were made from combinations of PLLA and PCL-DA (“Mod”). The membranes at 37° C. were rigid and not deformable. Once heated above 55° C., the tensile modulus of the membranes decreased dramatically, as shown in FIG. 18.

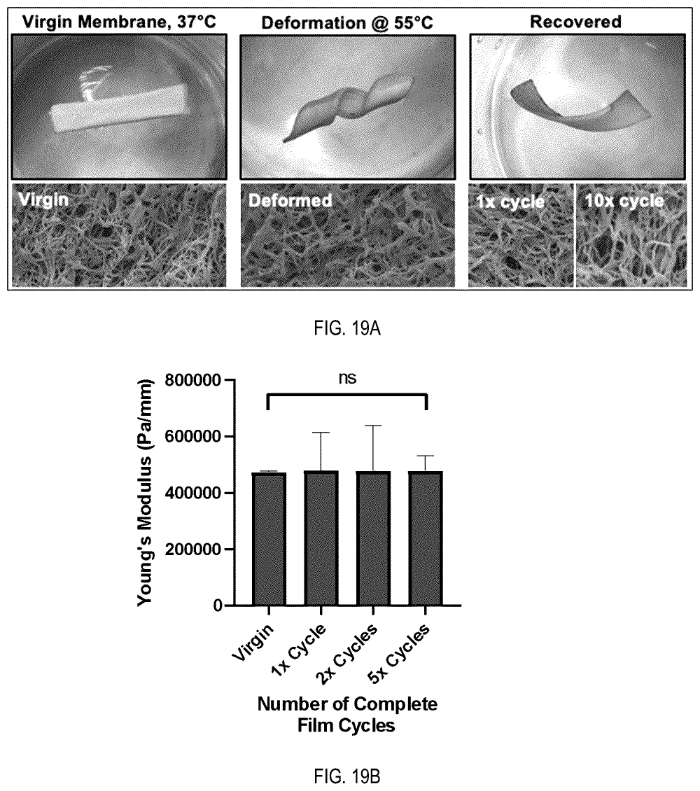

At a temperature of 52° C. or above, the membranes are easily deformed. Once cooled below 52° C. (i.e., 37° C.), the matrix retains its deformation and regains its rigidity. Upon heating above the 52° C. threshold again, the matrix recovers its original memorized structure. At the surface, deformed and recovered matrices maintain their nanofibrous surface topography, even after ten cycles of heat-deform-recover-repeat as shown in FIG. 19A. FIG. 19B shows the Young's modulus of the film sample as a function of cycling times and substantially did not change after repeated deformation-recovery cycles, even after 10 cycles. The test results demonstrated that the TS-MMS film samples did not fatigue and further did not lose the nanofibrous architecture.

The working times (shape memory recovery time) of these matrices at different temperatures of 50° C. and 80° C. were observed to be directly proportional to the incorporation amount of the PCL-DA, as shown in FIG. 20. PCL is the thermosensitive molecular switch in the matrix. Increasing the concentration of PCL-DA increases the number of potential molecular rearrangements which occur within the matrix and increased enthalpy reservoir, which ultimately represents a longer recovery time.

The membrane working time is affected by PCL-DA molecular weight, to a lesser extent. Decreasing the molecular weight of the PCL-DA resulted in an increased chain-extension and chain-end defects as shown in FIG. 21.

A decreased molecular weight allows for more homogeneous distribution of the molecular switches (the PCL polymer), and greater number of potential rearrangements within the matrix at a temperature of 52° C. or above.

These data corroborate with differential scanning calorimetry indicating that PCL-DA is responsible for TS-MMS behavior in an incorporation ratio-dependent manner.

### Example 9

Macropore Recovery in Macroporous Tissue Engineering Scaffold

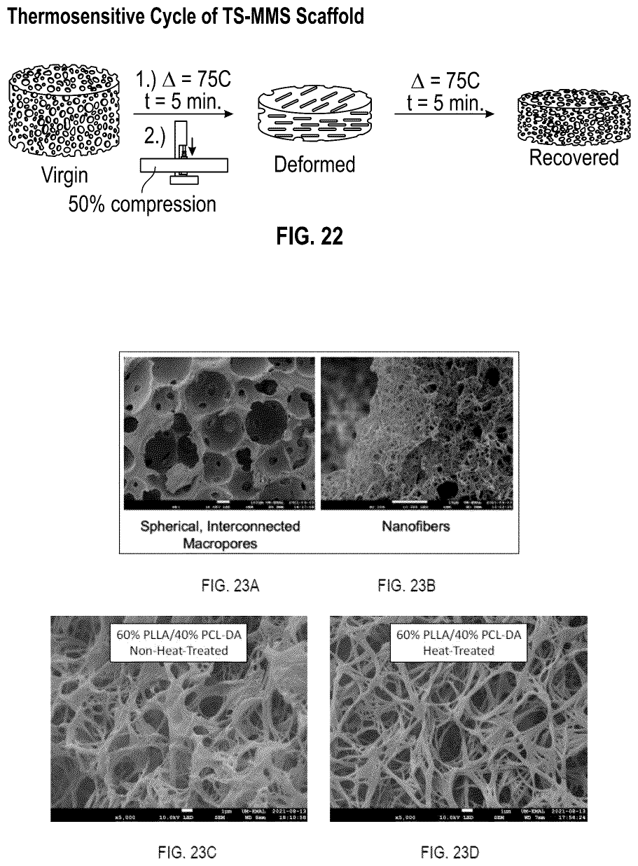

The thermosensitive cycle of the TS-MMS scaffold was tested to evaluate the pore recovery of the TS-MMS scaffold, as compared to the pure PLLA scaffold. The test process is shown in FIG. 22.

The nanofibrous macroporous scaffolds used in this study were fabricated from TS-MMS material as described in Example 5, as shown in FIGS. 23A and 23B. Without wishing to be bound by theory, it was hypothesized that compared to a macroporous scaffold fabricated form PLLA, the TS-MMS material having PLLA/PCL would impart a temperature-regulated resiliency of the pores to better accommodate clinical handling. Pores are a key consideration in tissue engineering constructs to enable cell infiltration and migration, tissue integration, vascularization, and nutrient/waste mass transfer.

The PCL-DA polymer, in solution with PLLA, is cast around the sugar spheres followed by chain-extension of the PCL-DA polymer chains. In this way, the memorized microstructure of the construct conforms to the macroporous architecture after the sugar sphere porogen is dissolved by water:

The PLLA nanofibers are the result of thermally induced phase separation via spinodal decomposition by a critical mass of PLLA homogeneously dispersed through the matrix, as shown in FIGS. 23C and 23D. The SEM micrographs illustrate the effect of heat treatment on nanofibers. The SEM micrograph of the nanofibers without heat treatment is shown in FIG. 23A and the SEM micrograph of the nanofibers with heat treatment is shown in FIG. 23B. The Macropores result from the sugar sphere porogen method. The macropore memory is the result of the formed IPN or SIPN of the PLLA polymer chains and the coupled PCL-DA polymer chains, in which the PCL-DA is chain-extended in the three-dimensional sugar sphere template, and interpenetrated by PLLA polymer chains or nanofibers.

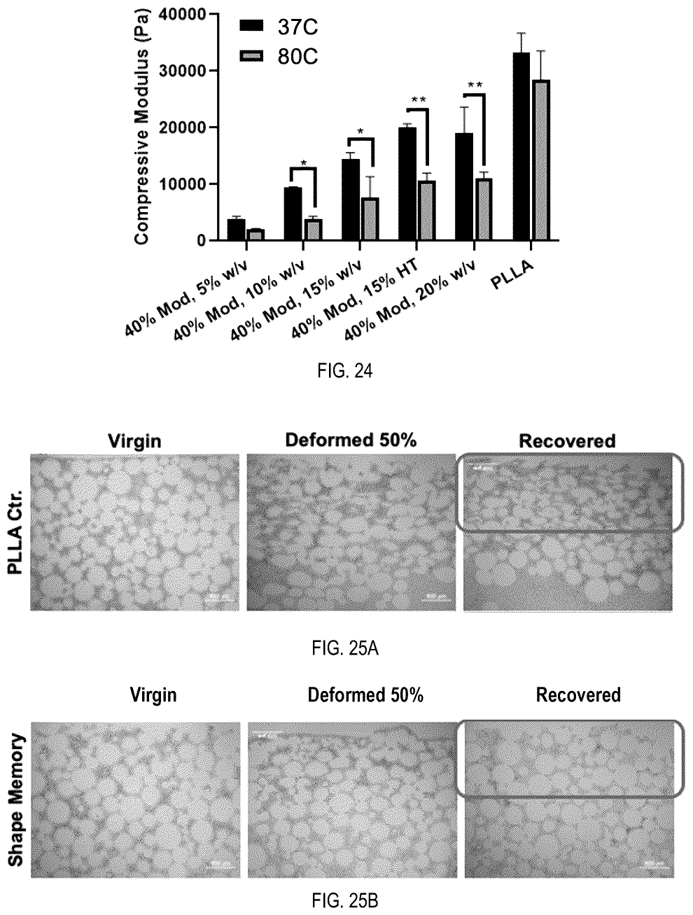

The ideal tissue engineering scaffold is sufficiently rigid at physiologic temperature to maintain its structural integrity, particularly considering forces experienced in the mouth. On the other hand, it should allow for clinical manipulation sufficient to accurately place and adapt the scaffold to an irregularly shaped defect. The TS-MMS scaffolds at various compositions have a tunable compressive modulus which is significantly decreased when the temperature is higher than 52° C., while the compressive modulus of PLLA does not significantly change as a function of temperature, as shown in FIG. 24. Additionally, the experimental results demonstrated that heat treatment of constructs after their fabrication allows for an increase in compressive modulus at physiologic temperature without a concurrent increase in compressive modulus at a temperature higher than 52° C., as shown in FIG. 24. Without intending to be bound by theory, it is believed that this increase can be attributed to partial phase separation of the PLLA from the PCL-DA within the confines of it's the chain extended polymeric network, such that it improves mechanical properties and amplifies the temperature sensitivity without modifying the nanofibrous architecture of the construct.

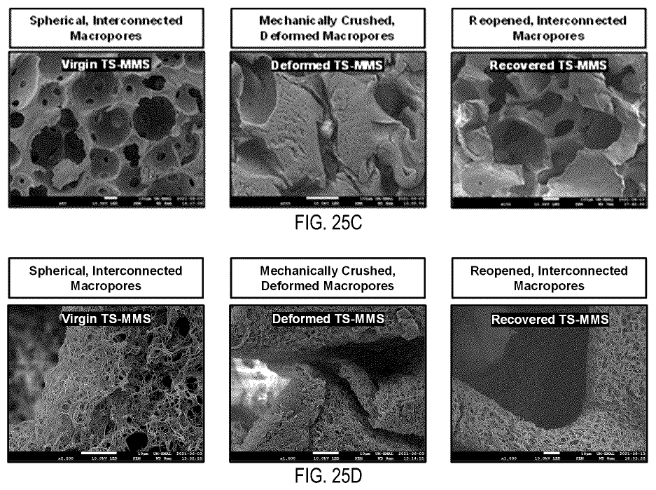

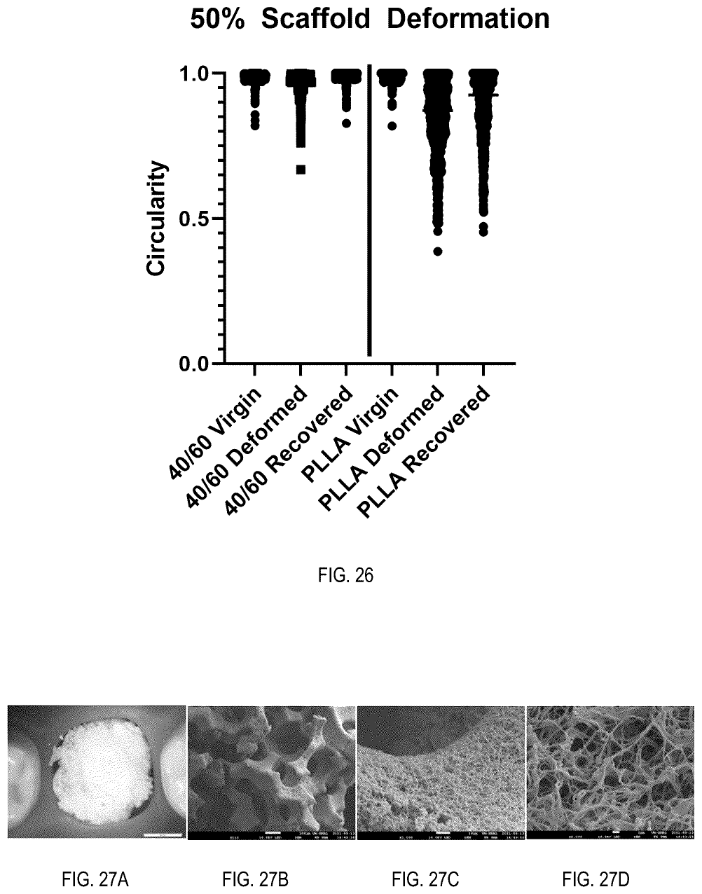

The TS-MMS scaffolds (40% PCL-DA/60% PLLA) and pure PLLA scaffolds were fabricated (“Virgin”) were separately heated to a temperature higher than 52° C. in a 55° C. warm water bath for 5 minutes, then subjected to 50% axial deformation by a mechanical testing apparatus and held at this deformation for an additional 5 minutes until cooled to room temperature (“Deformed”). The constructs were then placed in a 55° C. warm water bath for 5 minutes to allow for MMS recovery. The scaffolds (n=3 per group) were embedded in paraffin wax and histologic sections were prepared for representative analysis of the internal macropore morphology as shown in FIGS. 25A and 25B. The images of the histologic sections (n>100 sections per sample) were taken with a stereomicroscope and analyzed by measuring pore circularity as shown in FIG. 26. As shown in FIG. 25A, the pure PLLA scaffold pores were significantly flattened during deformation as shown in FIG. 25A (middle) as compared to the original pore size (left), and insignificantly changed by recovery at 55° C. (right). These occluded pores significantly hamper the ability of the scaffold to facilitate cell, tissue, and vascular ingrowth, and ultimately will lead to implant rejection and failure, which is detailed in Example 18 below. On the other hand, as shown in FIG. 25B, the TS-MMS scaffolds of the present disclosure demonstrated nearly full recovered after deformation (middle) and recovery (right) as compared to the original scaffold (left). These pores facilitate robust vascularization, cell and tissue infiltration towards accelerated and predictable healing owning to their enhanced clinical handling properties, which is detailed in Example 18 below. FIGS. 25C and 25D show the macropore recovery for the two TS-MMS scaffolds show in FIGS. 23A and 23B, respectively, using SEM images. The SEM images in FIGS. 25C and 25D also demonstrated nearly full macropore recovery after deformation (middle) and recovery (right) as compared to the original scaffold (left) for both TS-MMS scaffolds, which agree with the images of the histologic sections in FIG. 25B.

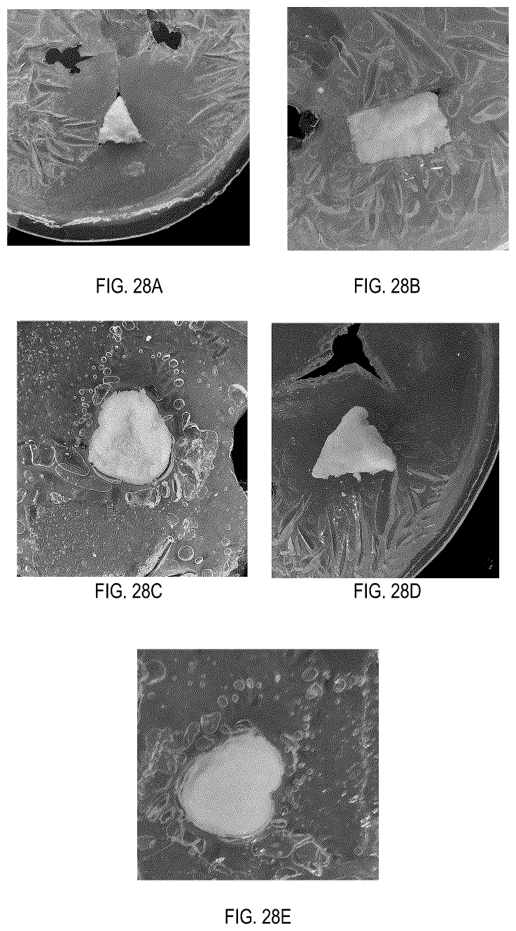

In one embodiment, this S-MMS scaffold can augment craniofacial bone healing in a cranial defect. In another embodiment, it can augment alveolar bone healing in the case of ridge preservation following tooth extraction (socket preservation). In another, it can be used as a space filling matrix for horizontal and/or vertical ridge augmentation, as shown in FIG. 27A. FIGS. 27B-27D show the SEM images of the macroporous structures of the scaffold. In another, the TS-MMS scaffold may be used as a space filling matrix for maxillary sinus augmentation. In another, it may be used as a space filling matrix in orthognathic surgery. It may also be used in conjunction with a dental implant to facilitate implant integration and periimplant regeneration. Applicants demonstrated in a mandibular molar extraction model ex vivo that the scaffold matrix may be adapted to an extraction socket when the temperature is higher than 52° C.; on cooling, the macropores expand to their original morphology maintaining the highly porous internal architecture of the scaffold and its nanofibrous surface morphology.

The TS-MMS scaffolds were tested and were able to fit various irregularly shaped defects after thermal induction and deformation. 15 mm diameter×2 mm tall cylindrical scaffolds were heated at 55° C. for 5 minutes then deformed to fit into irregularly shaped defects (simulated) cut from 4% agar plates kept at 37° C., as shown in FIGS. 28A-28E, respectively. The TS-MMS scaffolds demonstrated nearly full recovered pores after processing to fit into different irregularly shaped defects, as compared to the original scaffold.

### Example 10

Biologic Proof of Concept for Scaffold Macropore Recovery

Biphasic Morphology of Periodontal Membrane

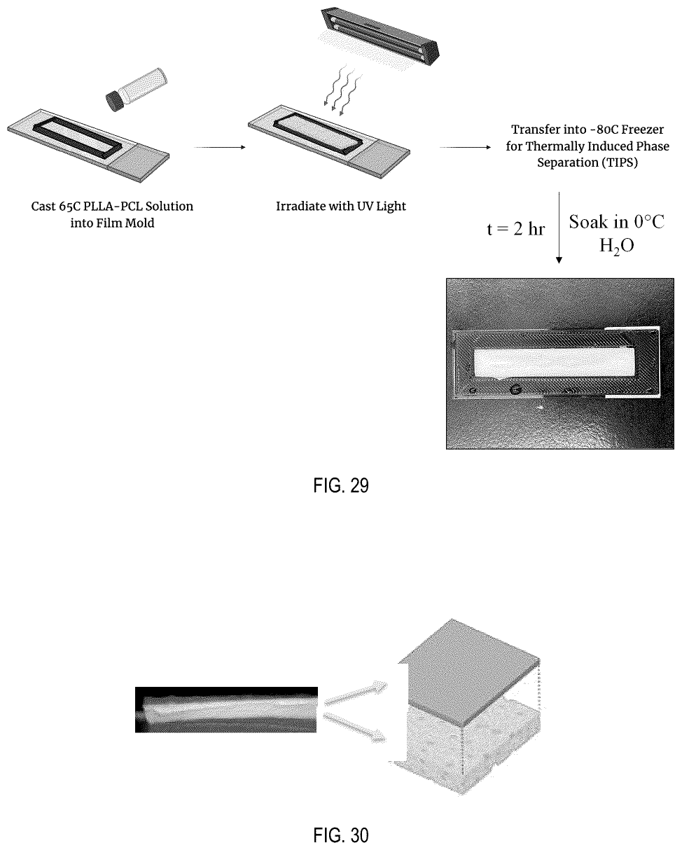

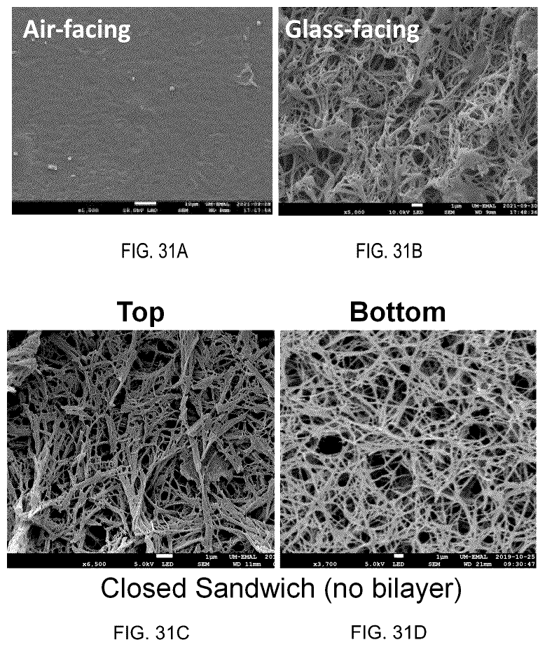

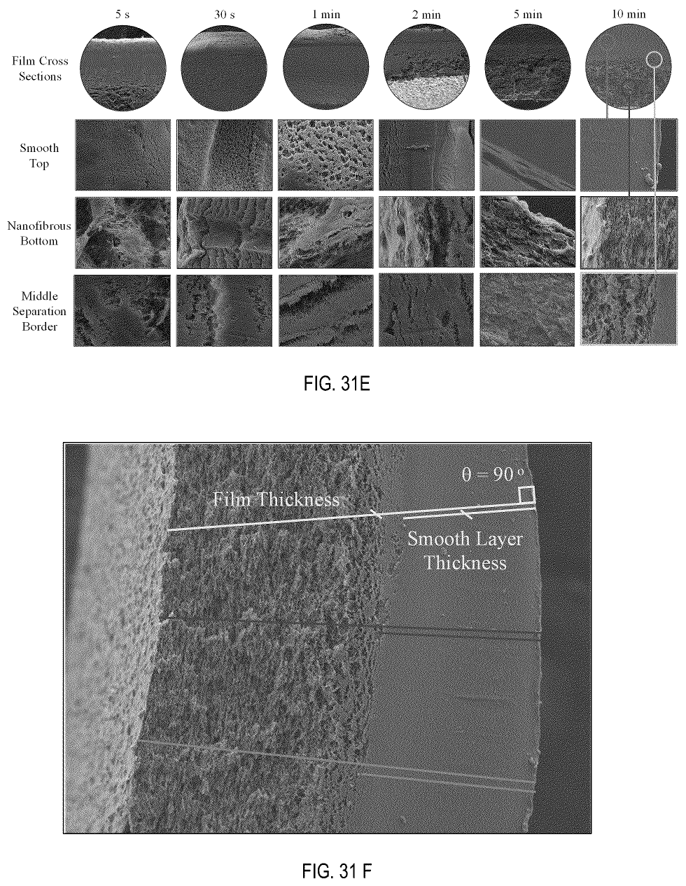

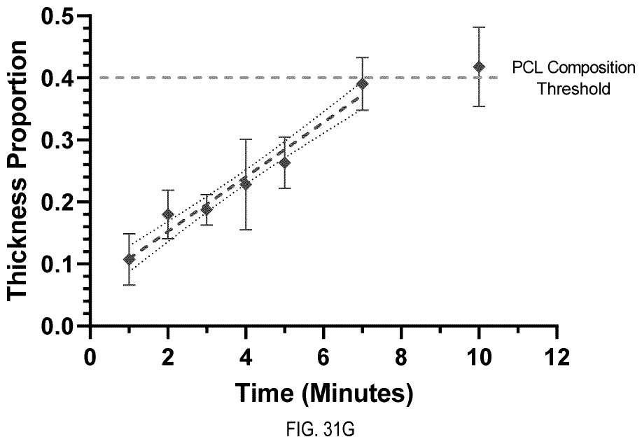

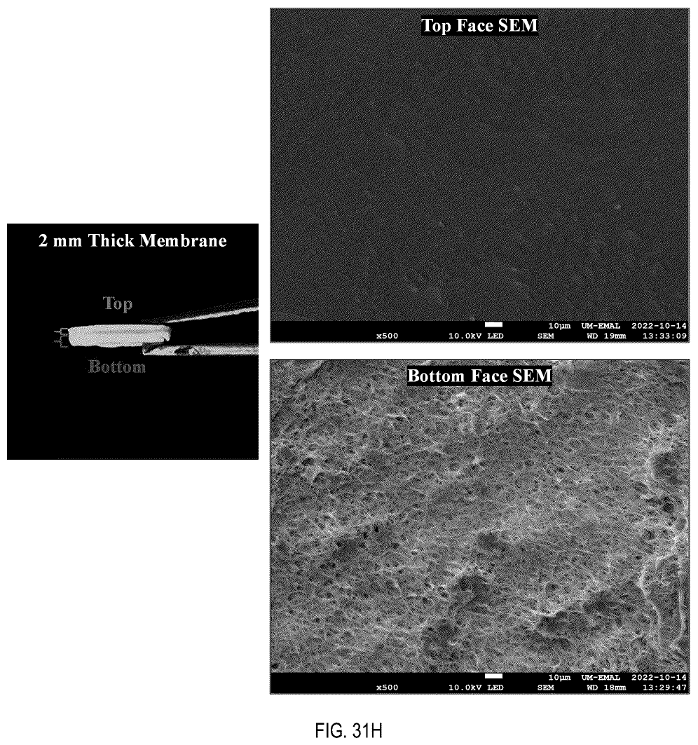

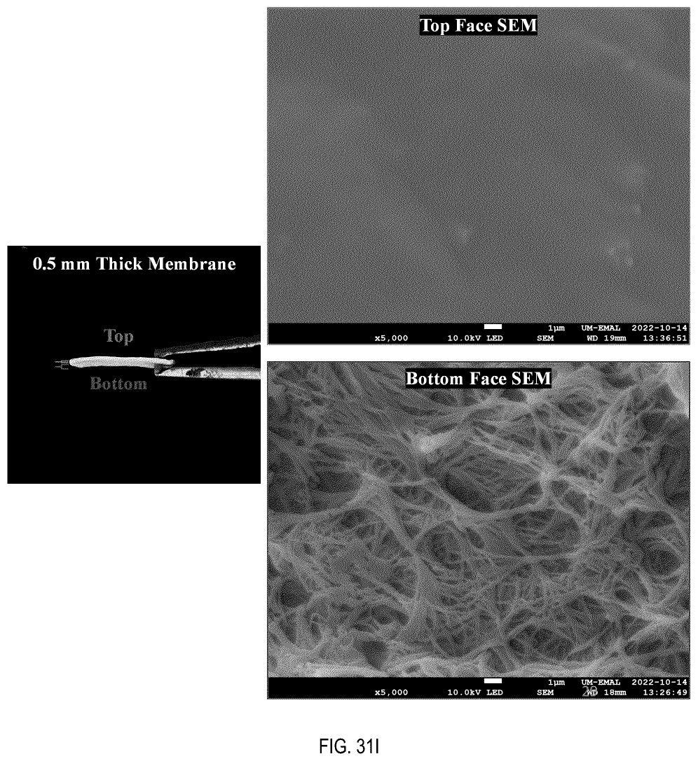

Owning to the novel “open-face” fabrication protocol of the present disclosure, as shown schematically in FIG. 29, the TS-MMS membranes have a biphasic morphology, as shown in FIG. 30. The layer of the glass-facing side is homogenously nanofibrous and is porous, resulting from the thermally induced phase separation of PLLA from THF solvent in the TS-MMS mixture. The layer of air-facing side results in a smooth “air-inhibited layer” whereby thermally induced phase separation is inhibited by the premature evaporation of THF solvent, as shown in FIGS. 31A-31C. The air-inhibited layer is a critical feature of the matrix, enabling its barrier properties.

On the contrary, the closed sandwich technique facilitates uniform nanofiber formation throughout the construct, as shown in FIG. 31D. As shown in FIG. 31D, both the top and bottom layers of the sample had the porous nanofiber structures by using the closed mold. For this application, the bilayer design is favorable. Solvent choice was previously described as being necessary to facilitate thermally induced phase separation. The thermally induced phase separation of crystalline PLLA from its organic solvent resulted in the formation of the nanofiber structure of PLLA polymer.

For the open sandwich technique, the thickness of the air-inhibited layer may be modulated by time of UV light curing, as shown in FIGS. 31E-31G. The SEM images in FIGS. 31E and 31G demonstrated the effect of crosslinking time (using open sandwich technique) on bilayer formation. A minimum time of 5 seconds of crosslinking is needed to induce a bilayer morphology of the test samples. For the TS-MMS film of PLLA/PCL (60/40), the thickness of the smooth PCL layer is proportional to the irradiation time by the UV lights, as shown in FIG. 31G. In FIG. 31G, the thickness proportion was the ratio of the thickness of the smooth layer to the thickness of the porous layer. This study demonstrates the effect of in-situ photopolymerization time on bilayer thickness. A maximum bilayer thickness is achieved by irradiation time of 8 minutes, upon which there is diminishing return in the thickness of the smooth layer. The smooth layer thickness may be modulated for specific applications.

The effects of the test film thickness on the bilayer formation and thickness were also studied. The SEM images of two films having thickness of 2 mm ND 0.5 mm are shown in FIGS. 31H and 31I, respectively. The test results demonstrated that membranes with the bilayer morphology may be formed at various thicknesses based on the template for the membrane and volume of polymer solution introduced in the photopolymerization, while maintaining the bilayer morphology.

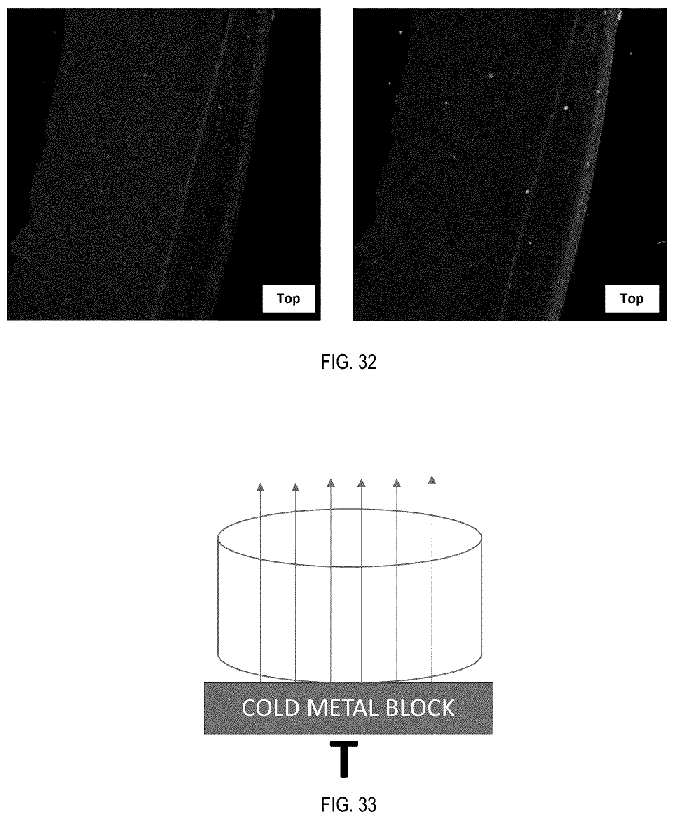

Applicants synthesized fluorescently labelled TS-MMS components to investigate the molecular dynamics of the novel fabrication process of the present disclosure. Nile blue-PLLA was synthesized by from an acrylic-terminal PLLA (HEMA-PLLA). Acrylic-end functionalized PLLA was synthesized from (hydroxyethyl)methacrylate (HEMA) initiator (0.8 mmol, 86 μL) and L-lactide monomer (40 mmol, 5.760 g), catalyzed by Sn(Oct)₂ (112 μL) in a ring opening polymerization at 120° C., in an inert N2 environment. At completion, the polymer was dissolved in 20 mL chloroform, and precipitated in 100 mL cold methanol (5× volume), where the product was collected by vacuum filtration, twice to remove unpolymerized monomer. HEMA-PLLA (1.40 g), nile blue acrylamide (0.012 mmol, 0.005 g), and freshly recrystallized azobisisobutyronitrile (AIBN, 0.06 mmol, 0.0098 g) were dissolved in 10 mL dioxane; the reaction was heated to 70° C. where it was allowed to proceed for 24 hours. Solvent was removed by rotary evaporation, the product was redissolved in a minimum amount of CHCl₃ and precipitated into cold methanol, then collected by suction filtration. Fluorescein-terminal PCL-DA (FITC-PCL-DA) was synthesized by the same method, where HEMA-PCL was reacted with FITC-o-acrylate, catalyzed by AIBN. Nile blue-PLLA and FITC-PCL-DA were incorporated at 5% wt/wt incorporation relative to each component (PLLA, PCL-DA). Following fabrication, membranes were sectioned perpendicular to their long axis and fluorescence was examined by confocal laser microscopy. Nile blue-PLLA (blue) is evenly distributed throughout the matrix below the air-inhibited barrier layer. On the other hand, FITC-PCL-DA is modestly distributed throughout the matrix below the air-inhibited barrier layer and concentrated within the air inhibited barrier layer, as shown in FIG. 32.

The barrier membrane capacity of TS-MMS periodontal membranes is the direct consequence of their chemical composition and fabrication protocol in such a way that the air-inhibited layer excludes tissue growth on one side, while the nanofibers facilitate tissue growth and augment cellular regenerative processes on the opposing side.

### Example 11

Oriented Nanofibers

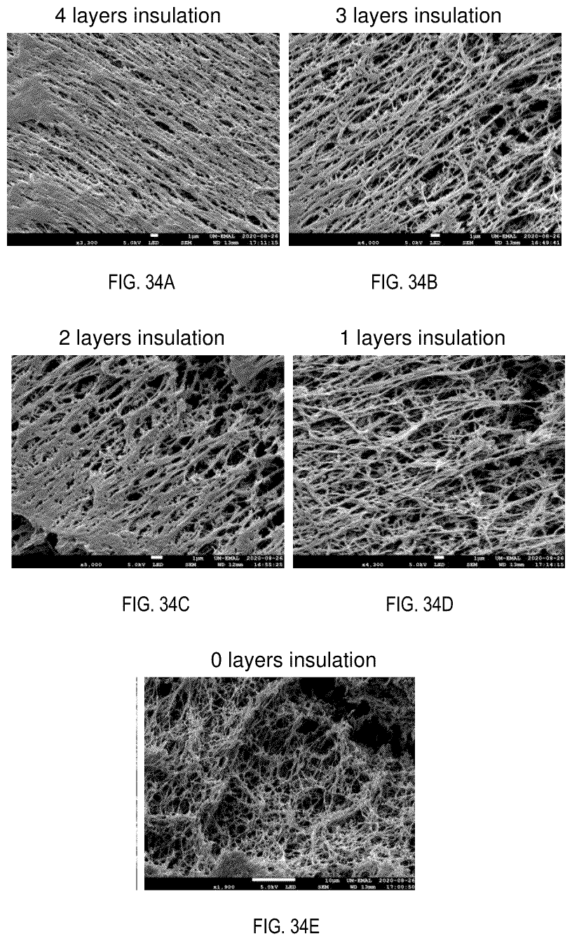

In one embodiment, the biphasic TS-MMS periodontal membrane of PLLA/PCL-DA can be fabricated with oriented nanofibers. The original fabrication protocol described is adapted by applying a uniaxial temperature gradient to the cast construct such that thermally induced phase separation of PLLA follows a directional gradient as shown in FIG. 33. The PLLA nanofibers are oriented substantially along the direction of the temperature gradient. The temperature gradient is applied through the combination of conducting (cold metal) and insulating (insulating tape) materials designed to apply the gradient, the SEM images of the nanofiber orientation as a function of the insulation layers are shown in FIGS. 34A-34E and FIG. 35. As shown in FIGS. 34A-34E, with more layers of insulation, the PLLA nanofibers are more oriented along the direction of the temperature ingredient. The SEM images of TS-MMS material with 4 layers insulation demonstrated the best PLLA nanofiber orientation along the direction of the temperature ingredient as shown in FIG. 35A, while with no insulation later, the PLLA nanofibers were not randomly distributed without orientation as shown in FIG. 35B.

### Example 12

Oriented Microtubes and/or Nanotubes

In another embodiment the oriented nanofiber fabrication protocol described above may be modified to accommodate nanotube or microtube formation. Rather than THF solvent, thermally induced phase separation of PLLA from benzene in a TS-MMS composite (5% wt/v, for example) allows for the formation of oriented microtubes and/or nanotubes. The microtubes and/or nanotubes are formed parallel to the axial temperature gradient or at an angle less than about 250 relative to the direction of the axial temperature gradient, as shown in FIG. 36A for SEM images of the vertical (parallel) section view and FIG. 36B for SEM images of the horizontal (perpendicular) section view. In cross sections (perpendicular to gradient), the tubular structure is appreciated.

### Example 13

Biphasic Degradation Behavior of TS-MMS (PLLA/PCL-DA (60/40))

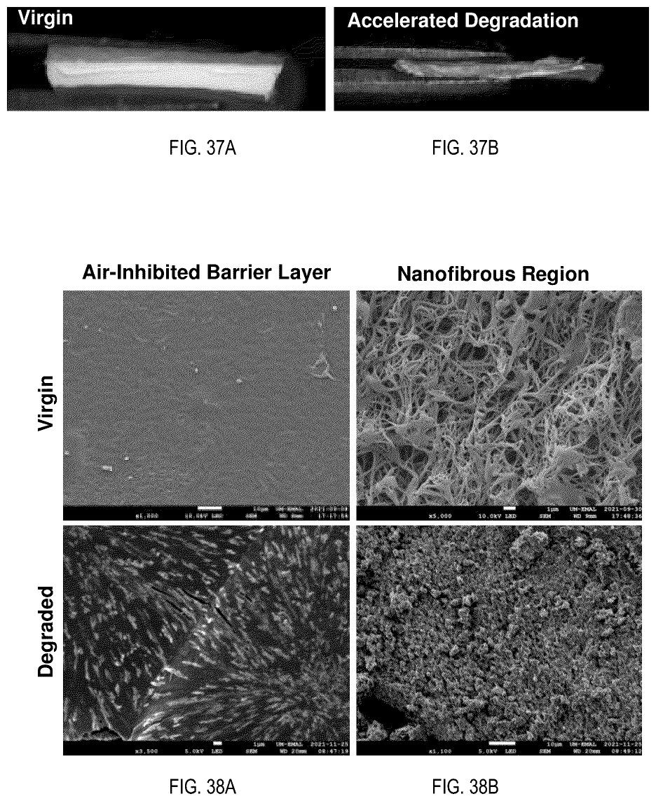

Based on the biphasic structure of the TS-MMS surgical membrane which is the direct result of its chemical composition and fabrication, Applicants hypothesized its biphasic degradation behavior, as shown in FIG. 37. The crosslinked PCL-enriched air-inhibited layer will degrade by polyester hydrolysis at a rate slower than PLLA-enriched nanofibers. The TS-MMS surgical membranes were fabricated in the PCL-DA/PLLA (40/60) formulation and subjected to an accelerated degradation test in 0.1 M NaOH. FIGS. 37A and 37B show gross microscopic images of the TS-MMS material before and after the degradation test, respectively. The test results demonstrated that the nanofibrous region (bottom in graphic) degrades quickly, while the air-inhibited barrier membrane degrades much more slowly, as shown in FIG. 37B.

Evaluation by scanning electron microscopy confirmed that the nanofibrous region (porous layer) degrades at a rate exceeding the air-inhibited barrier layer as shown in FIGS. 38A-38B. FIG. 38A shows two SEM images of the air-inhibited barrier layer before (top) and after (bottom) the degradation test, respectively. FIG. 38B shows two SEM images of the porous layer before (top) and after (bottom) the degradation test, respectively.

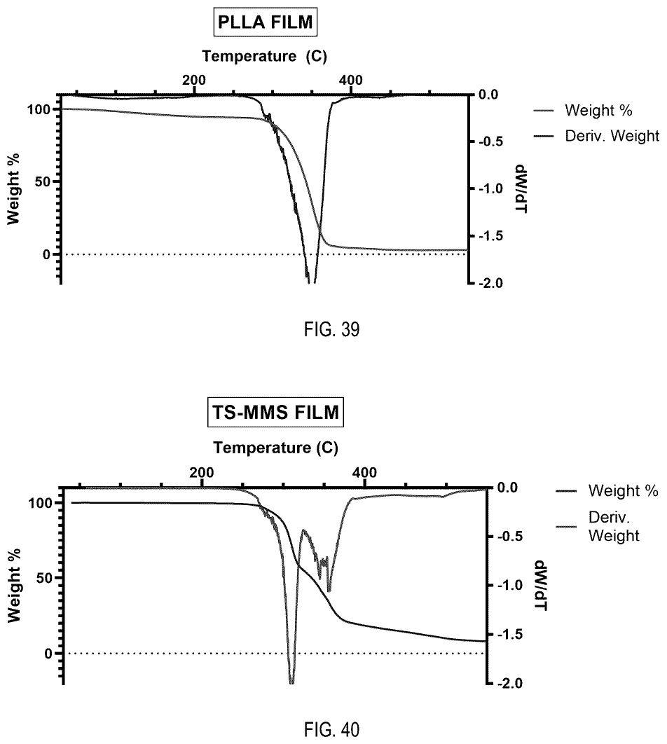

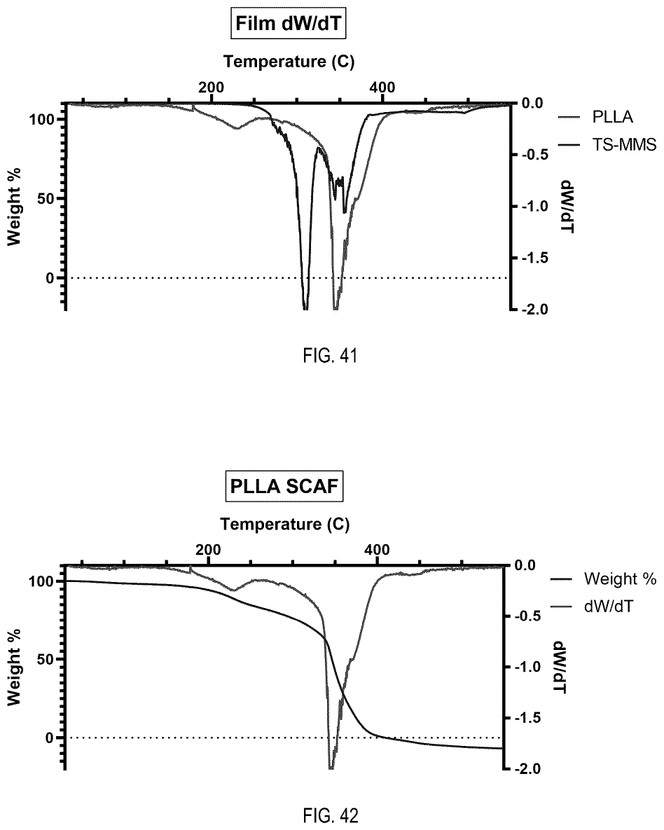

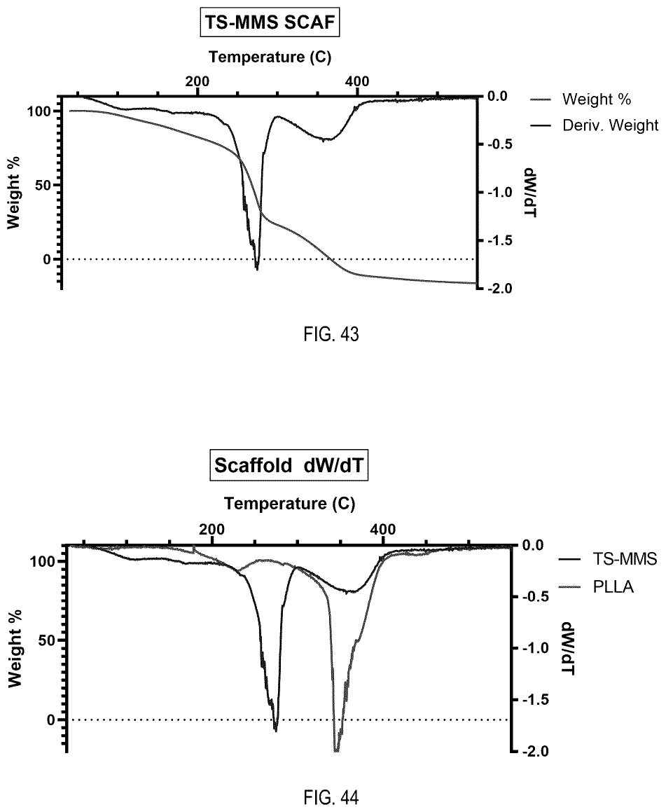

The biphasic degradation mechanism was confirmed by thermogravimetric analysis in the case of both scaffolds and membranes. The TS-MMS materials demonstrated a biphasic thermal degradation as compared to monophasic thermal degradation for PLLA (FIGS. 39-44), confirming that these properties are again unique to the TS-MMS composition but not fabrication method of the TS-MMS materials presented herein, as shown in FIGS. 41 and 44.

Thus, the TS-MMS surgical membrane is entirely biodegradable and bioresorbable. Its nanofibrous region degrades along with tissue formation, while the barrier membrane stays intact for a longer period to allow complete regeneration and remodeling to take place in the isolated defect site without epithelial downgrowth or external involvement. In various embodiments the degradation rate of each layer may be tuned by its chemical identity, crosslinking density, and molecular weight of the interpenetrating and network components.

### Example 14

Fixation of Periodontal Membrane

A key consideration for surgical membranes is their fixation within a defect site to maintain their desired location. The TS-MMS of the present disclosure lend themselves to surgical fixation by sutures, screws, and/or adhesives. Applicants demonstrated that TS-MMS films were easily suturable (for example, with 4-0 Ethilon monofilament sutures) and the suture was retained to a high suture pull-out strength, well above the requirements needed to withstand the oral environment.

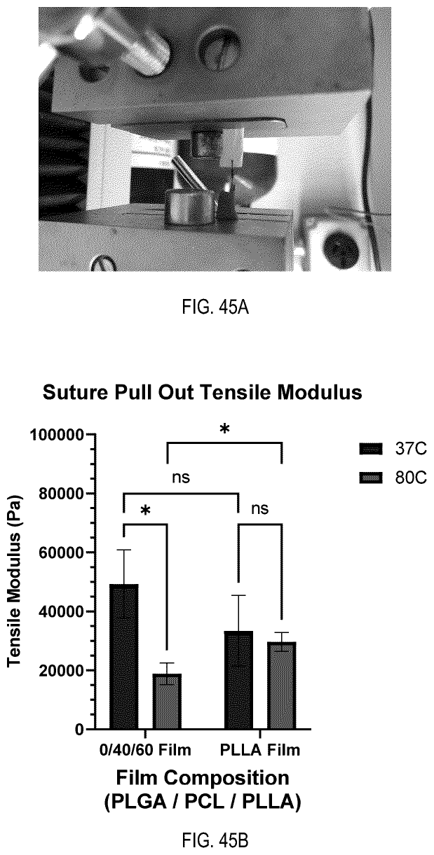

FIG. 45A shows a photograph of a mechanical testing apparatus used to assess suture retention strength from TS-MMS membranes. The suture pull out tensile modulus were measured for a TS-MMS film having PLGA/PCL/PLLA in a weight ratios of 0/40/60 and a PLLA control film, as shown in FIG. 45B. As shown in FIG. 45B, the suture pull out tensile modulus for the TS-MMS (PLGA/PCL/PLLA) film is significantly higher than that of the PLLA control film at the physiologic (body) temperature of about 37° C., indicating that the suture can be better retained within a tissue or defect site which is favorable to maintain the suture in and resist its pulling out; and is lower than that of the PLLA control film at elevated temperature of 80° C., indicating improved processability and being easy to suture into the tissue or defect site during the implantation process.

In another embodiment, TS-MMS membranes may be fabricated with a tissue-adhesive border through either inherent chemistry or adjunctive adhesive, in such a way that they are self-adhesive to a defect site through an adhesive border. The TS-MMS membrane functions as a patch to augment healing and regeneration with barrier membrane properties.

### Example 15

Cell Adhesion and Selective Integration of Periodontal Membrane

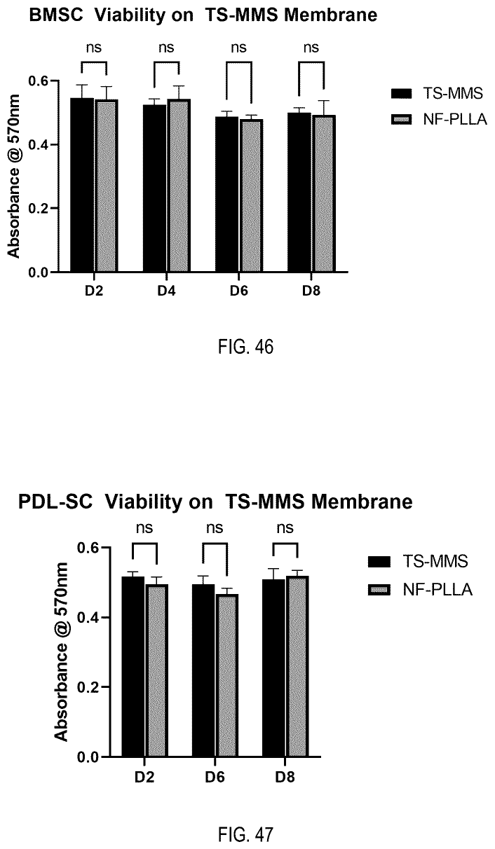

Applicants assessed the ability of the TS-MMS membranes (PLLA/PCL-DA (60:40)) of the present disclosure to facilitate cell adhesion, maintain stem cells within the matrix, and selectively permit cell integration (barrier function) using bone marrow stromal cells (BMSCs, FIG. 46) and primary periodontal ligament stem cells (PDL-SCs, FIG. 47)—the two major endogenous cell sources for periodontal regeneration. The TS-MMS membranes and pure PLLA membranes were sterilized by ethylene oxide gas and wet with 70% ethanol for 30 minutes in a tissue culture hood, prior to washing twice with cell culture media (DMEM+10% FBS+1% P/S) and seeded with 200,000 cells per 1 cm×1 cm matrix. The cells were cultured for 8 days, changing the media every two days. The PDL-SC and BMSCs adhered to the scaffold and were maintained in vitro equivalent to PLLA control, and no difference was noted between cell types.

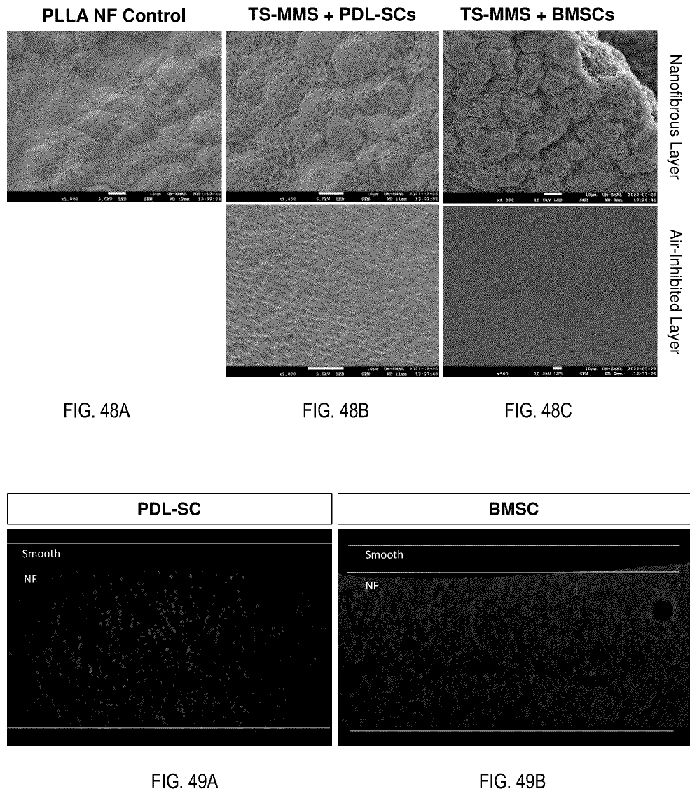

The membranes were seeded from both the nanofibrous and smooth sides, separately, to determine the ability of each side to contribute towards cell attachment and adhesion. For both PDLSCs (FIG. 48B) and BMSCs (FIG. 48C), the smooth side (bottom) did not facilitate cell adhesion, while the nanofibrous side (top) facilitated robust cell adhesion in TS-MMS comparable to PLLA nanofibrous control constructs (FIG. 48A), as shown in FIG. 48.

Finally, the constructs seeded from the nanofibrous side were subjected to histologic preparation by paraffin embedding and sectioning. The paraffin sections were treated with xylene and successive rehydration, then cells were stained using Phalloidin, a fluorescent marker of the cytoskeleton. Applicants demonstrate that cells robustly infiltrate the nanofibrous region, while even after 8 days culture in vitro the air-inhibited barrier layer retains its barrier function and prevents cell penetration or cell adhesion by confocal laser microscopy stained with AlexaFluor Phalloidin for both PDLSCs (FIG. 49A) and BMSCs (FIG. 49B), as shown in FIG. 49.

### Example 16

Embedded Controlled Release System in Macroporous Scaffold Matrix

Taking advantages of the high surface area of the TS-MMS matrices (PLLA/PCL-DA (60/40)) of the present disclosure, Applicants developed a protocol for the functionalization through the controlled release of nanofiber-embedded modalities. Previous methods to marry nanoparticles to a matrix rely on a solvent-wetting approach to loosely attach the nanoparticles at the matrix surface. The fabrication protocol of the present disclosure enables improved uniformity and functionalization of the scaffold compared to previous methods, notably full thickness functionalization of the scaffold rather than only the surface or the area near the surface.

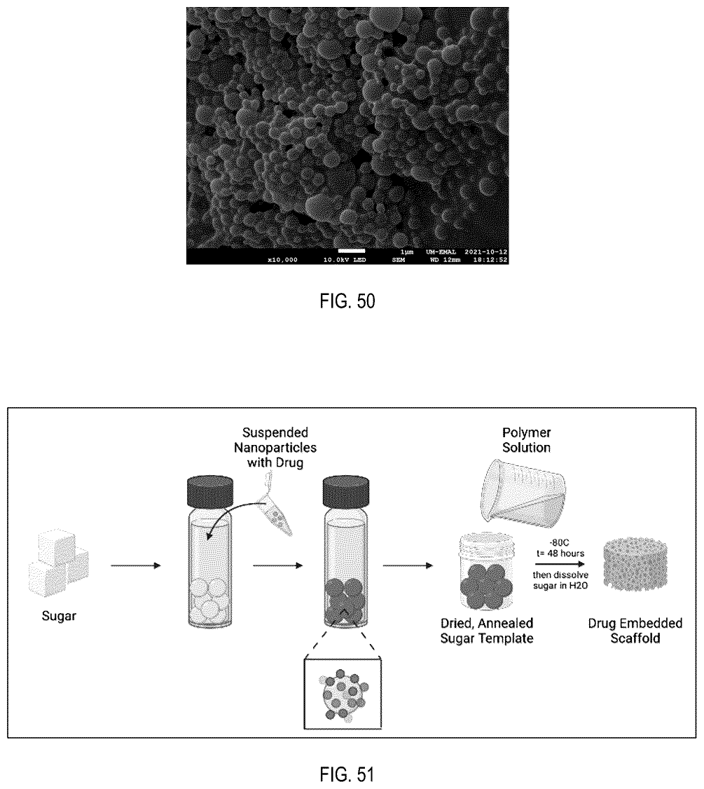

In this study, nanoparticles (NP) containing Rhodamine B, a fluorescent model molecule, were fabricated from poly (glycolic-co-lactic acid) (PLGA) via a double emulsion method, yielding PLGA nanoparticles (PLGA-NP), as shown in FIG. 50. An example of nanoparticle synthesis utilized approximately 5 mg of a molecule of interest, transferred into an Eppendorf tube where it was dissolved in double distilled water. The amount of the molecule of interest was variable depending upon the solubility in double distilled water. In a separate falcon tube, a solution of 5 mL dichloromethane and 50 mg of commercial 50:50 poly (D, L-lactide-co-glycolide) (MW=7 kDa-17 kDa) was prepared. The falcon tube was placed in an ice bath under the sonicator probe. Immediately prior to sonication, the Eppendorf tube solution was injected into the falcon tube. The resulting solution was sonicated at high intensity for 60 seconds. After this process, the sonicated solution was washed out with minimal water into a separate falcon tube containing 20 mL of 1% polyvinyl alcohol (PVA) aqueous solution (1% PVA in ddH₂O). This new solution was sonicated for 60 seconds and then transferred into a 300 mL glass beaker with moderate stirring. The falcon tube was washed with 3-5 mL of water three times and dumped into the beaker to maximize yield. The beaker was covered with foil and allowed to stir the particles overnight. After this time had elapsed, the contents in the beaker were transferred into ultracentrifuge tubes. PLGA-NP were recovered by centrifugation and subjected to several washing steps to remove residual surfactant and un-encapsulated molecules, prior to lyophilization. Their morphology was assessed by scanning electron microscopy.

Rather than attaching PLGA-NP to the scaffold material by solvent wetting, the PLGA-NP were added directly to sugar spheres fabricated by an emulsion of molten D-Fructose in mineral oil with Span80 surfactant, after their quenching and size selection by molecular sieves, in hexane, as shown in FIG. 51. The sugar-NP solution was agitated by sonic bath to ensure uniform distribution of PLGA-NP within the sugar spheres. In a 20 mL glass vial, 10 mL of hexane was injected prior to adding approximately 1.5 mL of sugar-spheres synthesized using the method described previously. This method is applicable for any size range of sugar-spheres so long as they are always kept under hexane to prevent clumping. In a separate Eppendorf tube, a desired weight of particles was added with 1.25 mL of hexane. Two drops of Span80® were added to the Eppendorf tube of particles, and this solution was vortexed until the particles are suspended and not clumped. The particle solution in the Eppendorf was immediately transferred into the glass vial of sugar-spheres. The sugar-spheres were agitated and stirred by hand with a transfer pipette to mix in the particles. This is performed until there were no observed clumps of particles and they appeared homogeneously distributed in the sugar. Once a homogenous distribution was observed, the sugar was used to make a scaffold template and biomaterial polymer was cast on the template following the methodology described above.

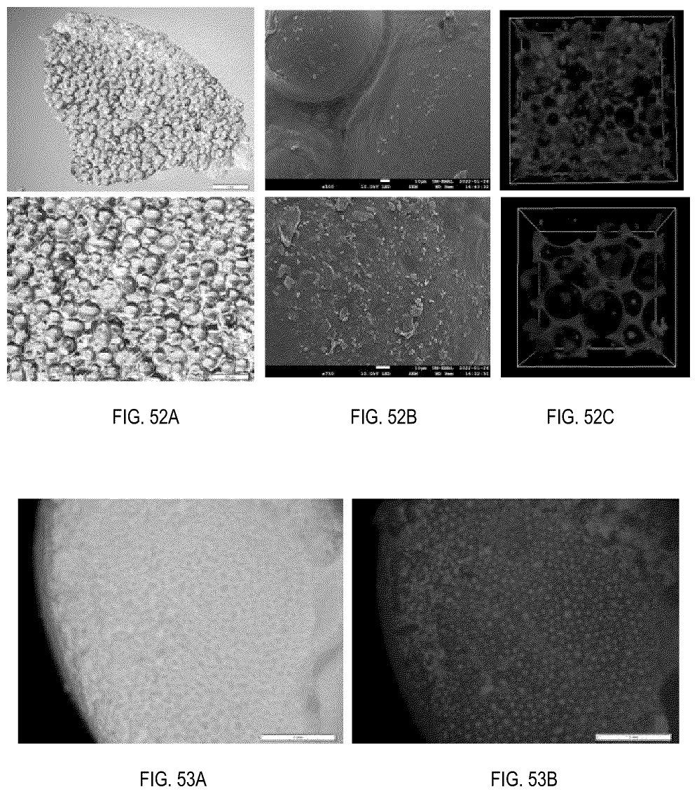

A known mass of particles was added to a known mass or volume of sugar spheres, designed to be fabricated in a Teflon mold of known volume. Hexane was removed to the height of the sugar spheres, and the sugar spheres were heat treated at 37° C. to cause their partial annealing, for a specified amount time (above: 5-15 minutes). Hexane was removed by vacuum evaporation, yielding a PLGA-NP laden sugar sphere template, shown in FIG. 52. The dried sugar template retained the PLGA-NPs throughout, as demonstrated by bright field microscopy (FIG. 52A, top and bottom in different magnifications), scanning electron microscopy (FIG. 52B) and three-dimensional confocal laser microscopy (FIG. 52C), respectively.

After the sugar template was dried, the TS-MMS or PLLA polymer in organic solvent were cast, augmented by vacuum. The matrix was kept at −80° C. for more than 48 hours to induce thermally induced phase separation, followed by processing in hexane for 24 hours to remove residual solvent and then in water for 24 hours to dissolve the sugar template. Resulting scaffolds retained the PLGA-NP which were transferred from the sugar sphere template to the polymer matrix during casting, as demonstrated by bright field (FIG. 53A) and fluorescence microscopy (FIG. 53B), respectively.

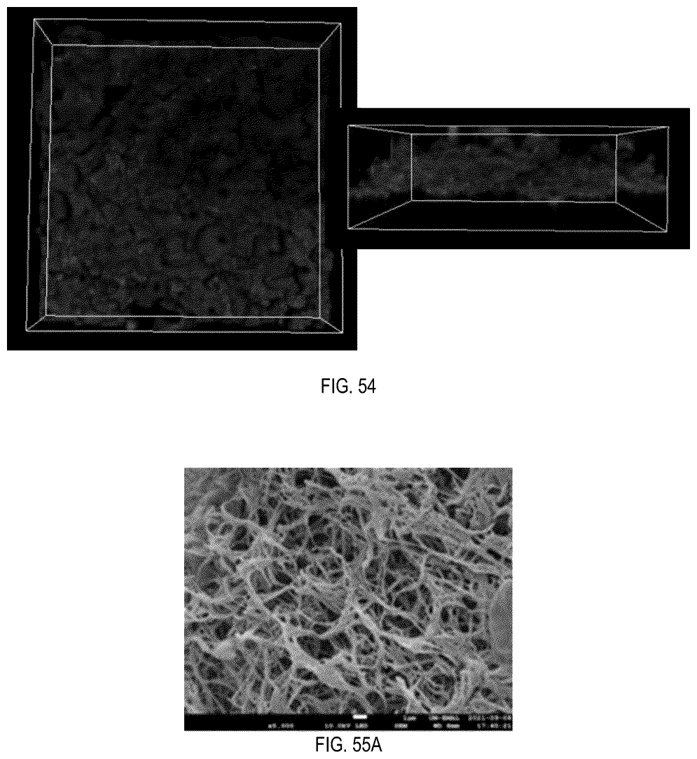

Three-dimensional confocal laser microscopy demonstrated uniform, full-thickness functionalization of TS-MMS scaffolds with PLGA-NP, as shown in FIG. 54. FIG. 54 shows three dimensional reconstruction of confocal laser microscopy images taken in a Z-stack of TS-MMS scaffold loaded with RhodamineB PLGA nanoparticles demonstrating their uniform incorporation into the macropore walls.

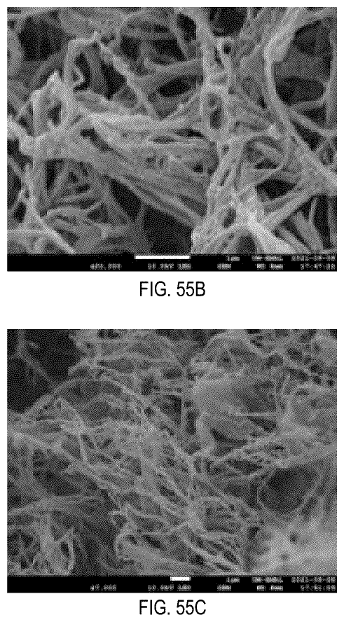

Scanning electron microscopy confirmed the abundance and uniform distribution of nanofiber-embedded PLGA-NPs, as shown in FIG. 55. The SEM micrographs in FIG. 55A-55C illustrate the nanoparticle-in-nanofiber (NP-NF) morphology of PLGA nanoparticles embedded into TS-MMS scaffold nanofibers.

### Example 17

Control Release Testing

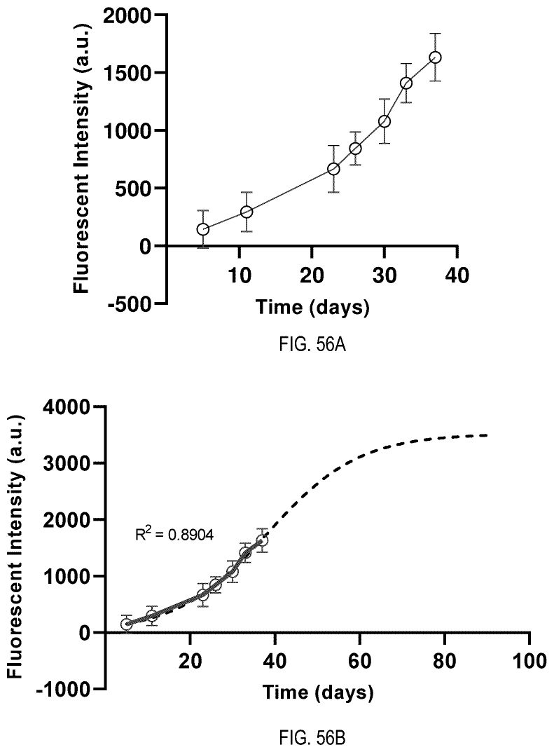

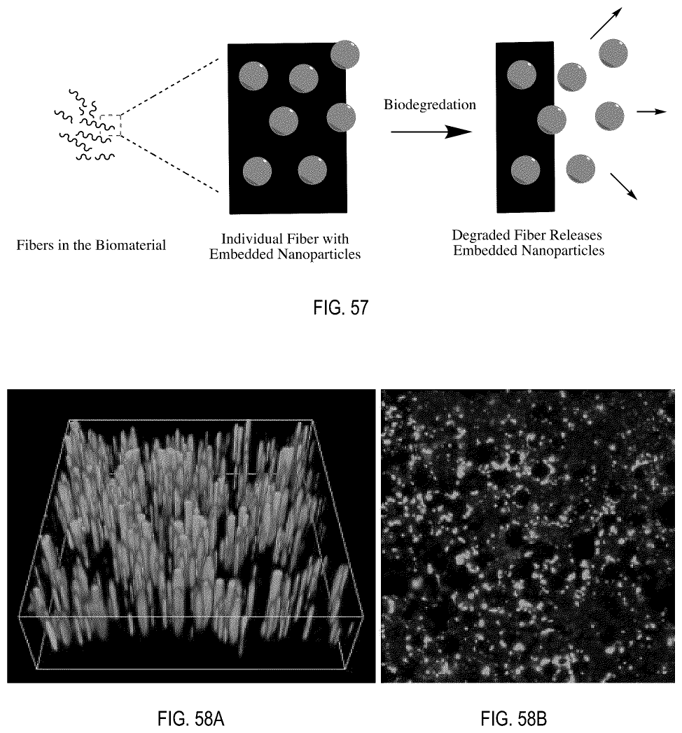

The nanofibrous TS-MMS scaffolds (PLLA/PCL-DA (60:40)) fabricated with PLGA-NPs containing rhodamine (17 samples) were subjected to a release test to determine the kinetics of controlled release. The scaffold constructs were incubated in a known volume of phosphate buffered saline (PBS) at 37° C. At various time points, the media was collected for spectrophotometric and fluorometric analysis and replaced with fresh PBS. The release is plotted as cumulative release over time as shown in FIG. 56A. FIG. 56B shows the model drug (Rhodamine B) release kinetics from scaffold samples (n=17) with a nonlinear regression curve fit for logistic growth. As compared to the free standing PLGA-NP (nanoparticles), the controlled release of rhodamine (or any drug substance in other applications) from the nanofibers of the TS-MMS scaffolds represents an advantage of controlled release by reducing the PLGA-NP exposed surface area to the bio-environment such that the change in surface area-to-volume ratio over time is less significant, resulting in a more linear release profile, as shown schematically in FIG. 57.

In another example, fluorescein isothiocyanate-labelled bovine serum albumin (FITC-BSA, MW: 66 kDa) was encapsulated in PLGA by a double emulsion method, and stabilized by 0.1% gelatin and/or 30 uM (+)-Trehalose. The TS-MMS macroporous scaffolds were fabricated with FITC-BSA PLGA-NP incorporated as a proof of concept for protein delivery from the scaffold and examined by confocal laser microscopy, as shown in FIG. 58.

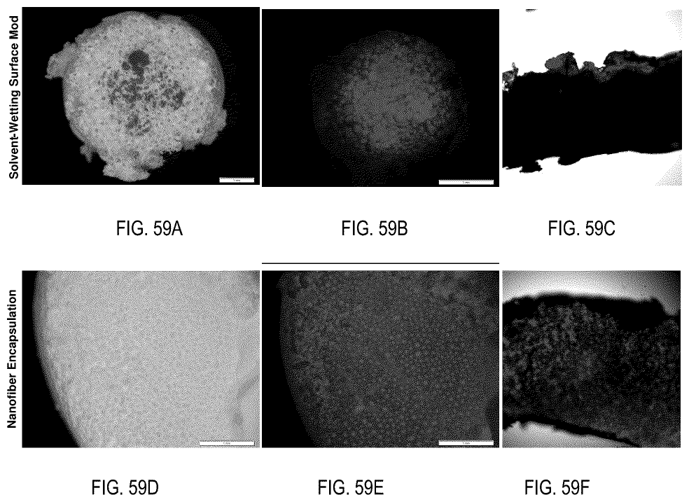

The TS-MMS functionalized with PLGA-NPs by the new nanofiber encapsulation method of the presented disclosure were compared with that prepared by the existing solvent-wetting approach of surface modification. The comparison results are shown in FIGS. 59A-59C for the scaffolds made from existing solvent-wetting approach, and in FIGS. 59D-59F for the scaffolds of the present disclosure, respectively. The bright field (FIG. 59A for control, and FIG. 59D for test sample of the disclosure) and fluorescence (FIG. 59B for control, and FIG. 59E for test sample of the disclosure) microscopy images of bulk scaffolds from the top view demonstrate that the nanofiber encapsulation method results in superior uniformity. The cross sections of each scaffold were taken at the center and imaged by confocal laser microscopy and shown in FIG. 59C for the control sample and in FIG. 59F for the test sample, respectively. In the cross section it is apparent that the solvent wetting approach only results in surface modification as shown in FIG. 59C, while the nanofiber encapsulation approach results in full thickness functionalization as shown in FIG. 59F. It is critically important to facilitate uniform, predictable regeneration in a critical sized defect.

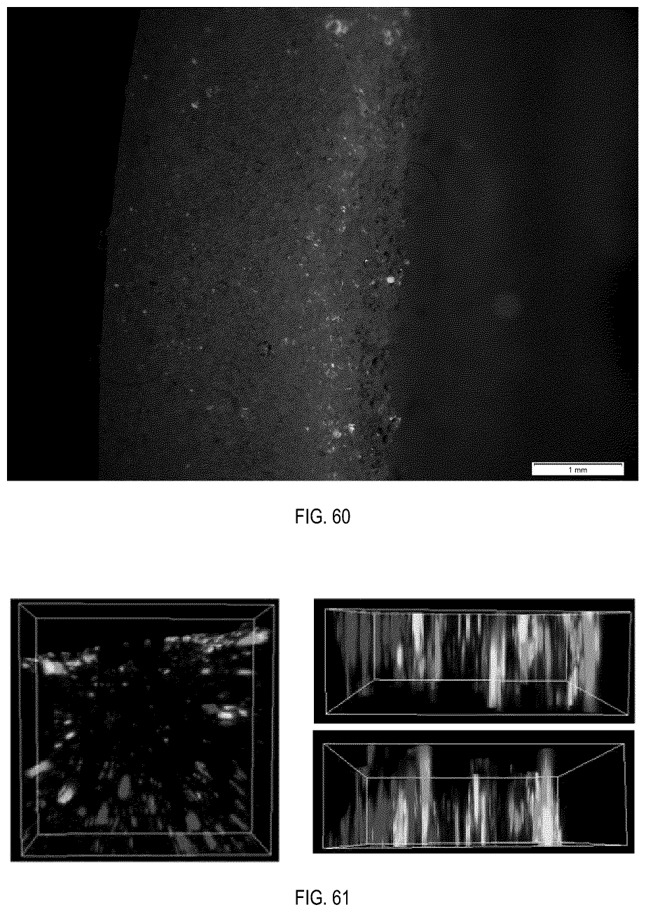

This method also allows for multiple unique cargos (in terms of encapsulated cargo, particle composition/degradation properties, or both) to be incorporated into a single construct with spatial specificity, shown as proof of concept in FIG. 60. To demonstrate this capability, FITC-BSA PLGA-NP was combined with sugar spheres to create the first layer of the scaffold, followed by annealing and solvent reduction, then a second layer of sugar spheres with RhodamineB PLGA-NPs was added and likewise annealed. The sugar template and scaffold were imaged by fluorescence microscopy and confocal laser microscopy, respectively, in FIG. 60 and FIG. 61.

In one embodiment, nanoparticles (5-1000 nm diameter) are embedded within nanofibers by the method described. In another embodiment, microparticles (1-10 um diameter) are embedded within nanofibers by the method described. Herein both the nanoparticles and microparticles are described as “particle”. In one embodiment, the particle is fabricated from a biodegradable polymer, such as poly (glycolic-co-lactic acid) (PLGA) by a double emulsion method. In another embodiment, the particle may be made by a microfluidic emulsion method. In another embodiment, the particle is synthesized by in situ polymerization of 2-hydroxyethylmethacrylate and other methacrylate monomers. In another embodiment, biologic substances are directly embedded in nanofibers by the method described whereby surface coating of the fabrication substrate with the biologic substrate allows its direct transfer into the nanofiber upon TS-MMS casting. In another embodiment, nanocarriers such as clay or carbon nanotubes are entrapped within nanofibers by the same method described. The kinetics of controlled release are influenced by TS-MMS composition, molecular weight, and crosslinking density. The kinetics of controlled release are also influenced by particle composition, molecular weight, and crosslinking density. For example, PLGA may be modified by modulating the lactide:glycolide ratio to increase or decrease hydrophilicity, to increase or decrease the rate of sustained release, respectively. Considerations for the encapsulated cargo are described below.

### Example 18

Embedded Controlled Release System in Periodontal Membrane

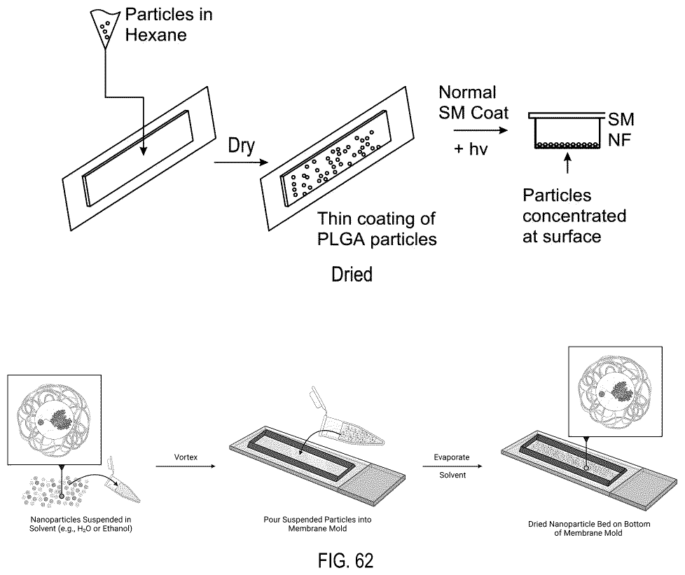

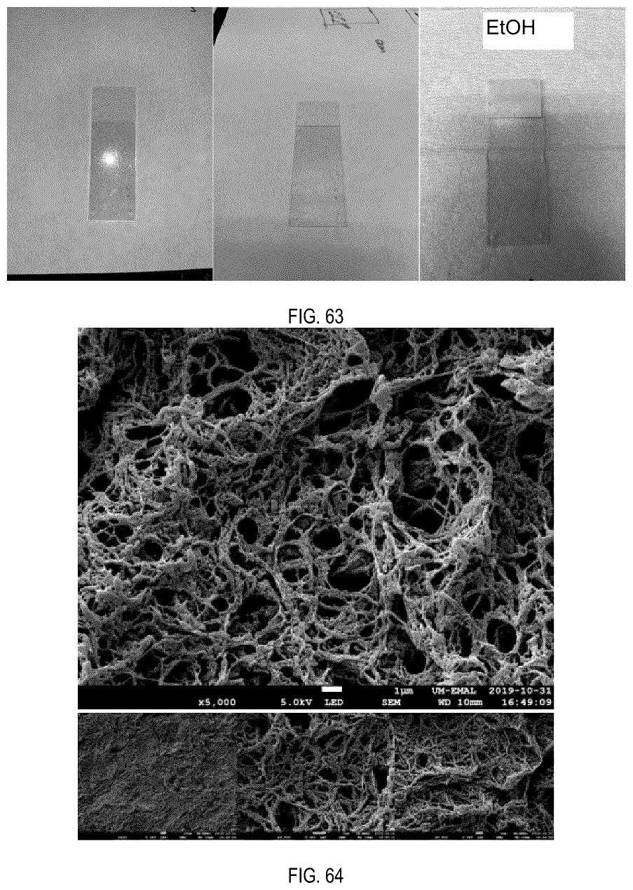

The PLGA-NP and other particle carriers to augment the healing process may also be incorporated into TS-MMS membrane nanofibers by a similar process. The PLGA-NPs were fabricated as described above, for example. In an Eppendorf tube, a desired weight of particles was added with 1.25 mL of ethanol. One drop of Span80® was added to the Eppendorf tube of particles, and this solution was vortexed until the particles are suspended and not clumped. The particle solution in the Eppendorf tube was immediately transferred to the charged side of the microscope slides with attached 3D film molds, prepared by the methodology described previously. This fabrication protocol was shown schematically in FIG. 62. The ethanol was allowed to dry, leaving behind a homogenous thin layer of particles on the microscope slide, shown in FIG. 63. These slides were then viable to undergo polymer casting for the synthesis of the TS-MMS films, also previously described in the methodology.

TS-MMS surgical membranes with nanofiber-embedded PLGA-NPs were evaluated by scanning electron microscopy which demonstrated uniform distribution and abundance of PLGA-NPs in the nanofibers of TS-MMS, as shown in FIG. 64.

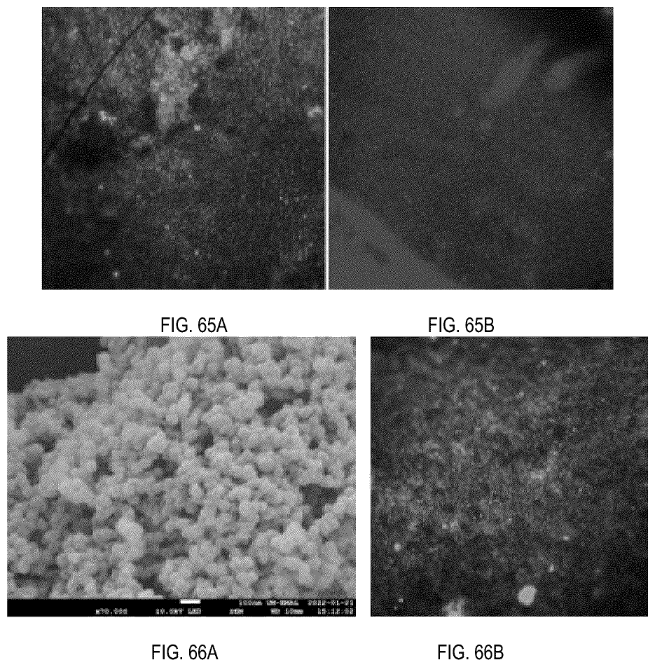

TS-MMS surgical membranes were fabricated with FITC-BSA PLGA-NP and RhodamineB PLGA-NP separately, as shown in FIGS. 65A and 65B, respectively. Applicants demonstrate that both FITC-BSA PLGA-NP (left) and Rhodamine B (right) PLGA-NP are efficiently loaded, visualized from the NF side by confocal laser microscopy. The FITC-BSA is a model drug of a protein therapeutic (e.g., growth factor) and the RhodamineB is a model drug for a small molecule therapeutics.

In another embodiment, nanoparticles are synthesized by in situ polymerization of 2-hydroxyethyl methacrylate, ethylene glycol diacrylate and fluorescein-o-acrylate, as shown in FIG. 66. The P(HEMA) nanoparticles are fabricated by in situ polymerization and observed by SEM (FIG. 66A). The P(HEMA) nanoparticles incorporating 5% FITC-o-acrylate (green fluorophore) are incorporated into TS-MMS nanofibers by the same method described for PLGA NPs and the nanofibrous layer is visualized by confocal laser microscopy, as shown in FIG. 66B.

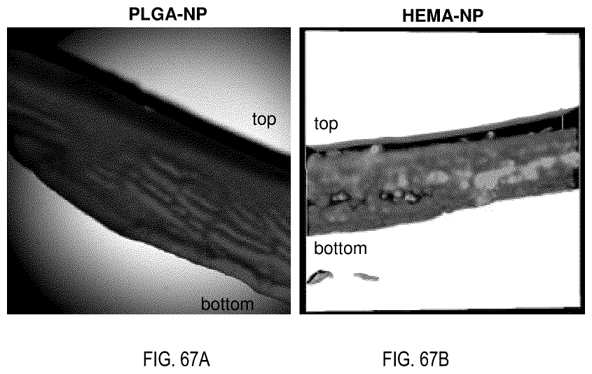

A unique feature of the proprietary fabrication strategy and composition of TS-MMS films containing particles of the present disclosure is their resulting directional release. As shown in FIGS. 67A and 67B, it was demonstrated that PLGA-NP (FIG. 67A) and HEMA-NP (FIG. 67B) localize within the nanofibrous layer (labelled as bottom) and not the air-inhibited layer (labelled at top).

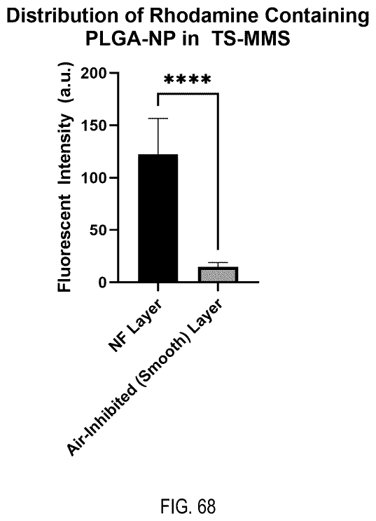

To confirm this observation, the air inhibited and nanofibrous layers from TS-MMS membranes fabricated with RhodamineB PLGA-NPs were isolated subjected separately to degradation in 0.1M NaOH at 37° C. for one hour. The resulting digests were assayed by fluorescence spectroscopy and demonstrate significant concentration of RhodamineB (from RhodamineB PLGA-NP) in the nanofibrous layer rather than air-inhibited layer, as shown in FIG. 68. As a result, this design motif which enables uniform distribution of controlled release vectors within a highly porous nanofibrous region, separated from its environment on one side by an impermeable barrier which is slower to degrade and lacks significant concentration of controlled release vectors, enabling directional release into the defect site.

Clinically, growth factors used in periodontal regeneration (i.e., PDGF-bb, Gem21S from Lynch Biologics) are released from the defect soon after delivery. Thus, the materials of the disclosure advantageously provide a means of providing a sustained dose and retaining the growth factor specifically in the defect site.

### Example 19

Introduction of PLGA to the TS-MMS Membranes and Scaffolds

The TS-MMs membranes and scaffolds having PLGA-DA and PLGA-TA were prepared in Examples 3-6. The purpose of introducing PLGA-DA or PLGA-TA to the TS-MMS membrane or scaffold is to modulate the degradation time, while maintaining the thermosensitive properties of these membranes or scaffolds. The TS-MMS material of PLLA/PCL may be suitable for long term drug delivery applications because both the PLLA and PCL polymers have long degradation time of over 24 months and approximately two to three years, respectively. The TS-MMS material may require different degradation rates for different applications. The degradation rate of the biodegradable polymeric material can be altered by adding other suitable biodegradable polymers such as PLGA which has a tailorable degradation time from about a few weeks to several months by varying the poly(lactic acid) to poly(glycolic acid) ratio. The PLGA polymer can be used to replace part or all of the PCL polymer as detailed in Examples 3 and 4 above.

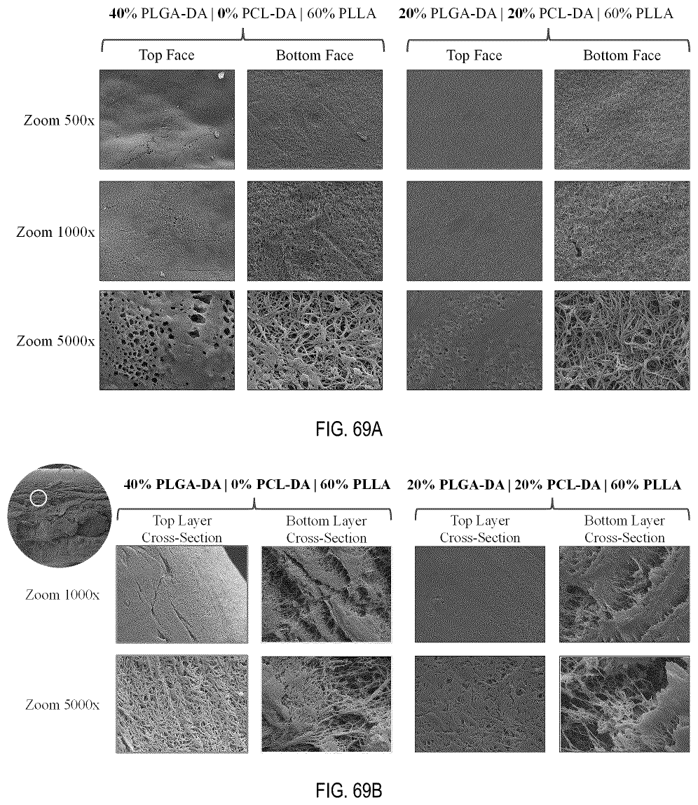

The effects of introduction of PLGA-DA to the TS-MMS materials on the morphology and physical properties were studied, and the test results are shown in FIGS. 69-76 respectively.

Impact on Bilayer Morphology of the TS-MMS Films Having PLGA

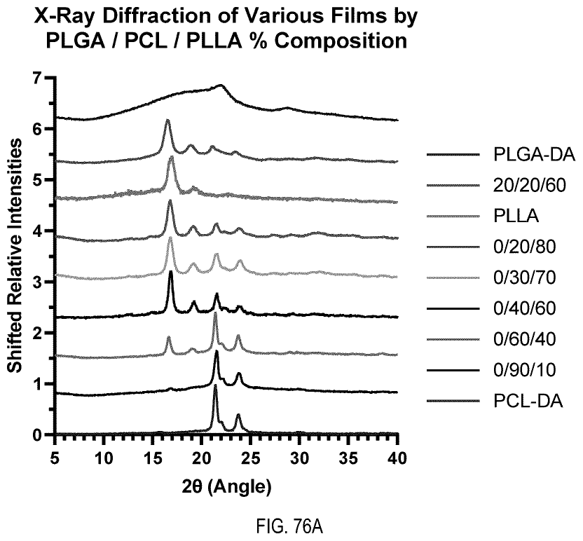

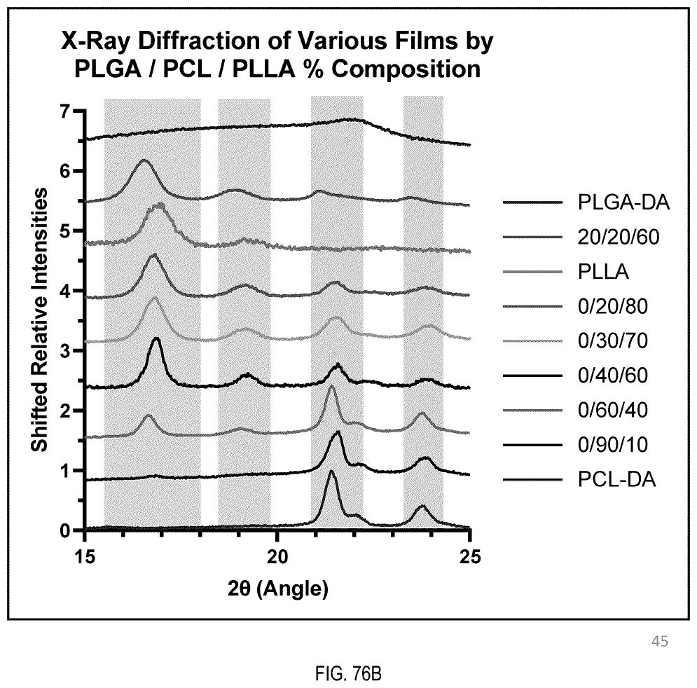

The SEM images of the top face and bottom face of the TS-MMS film are shown in FIG. 76A. The SEM images of the cross-section of the top layer and the bottom layer are shown in FIG. 76B. As shown in FIGS. 69A and 69B, the introduction of PLGA-DA to the TS-MMS material did not change the morphology of the bilayer components of the TS-MMS material.

Impact on Contact Angle of the TS-MMS Films Having PLGA

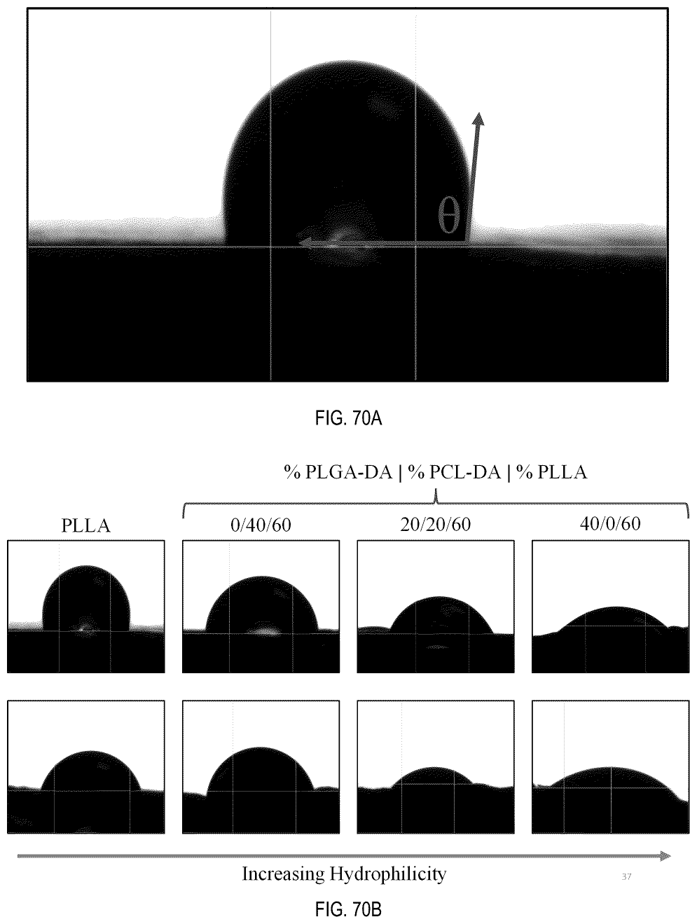

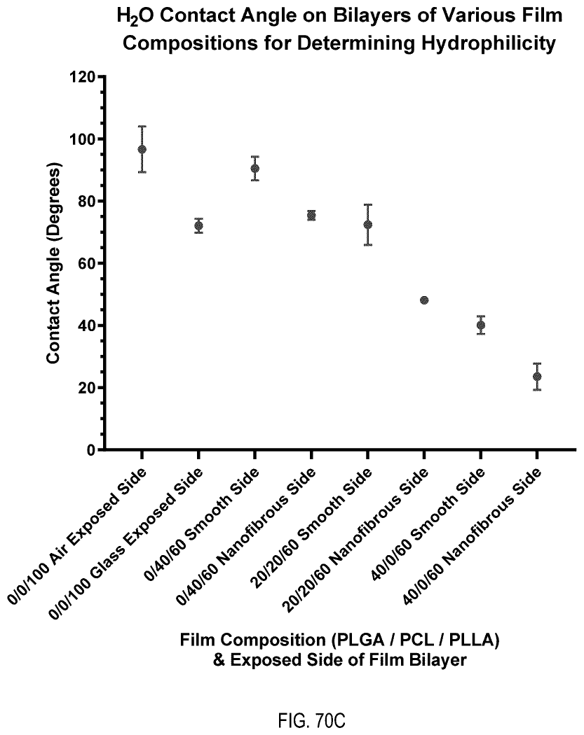

Introduction of PLGA caused a decrease in water contact angle with the TS-MMS film, reflecting increased wettability, as measured by a goniometer and quantified by image processing, as shown in FIGS. 70A-70C. It is advantageous to have a lower contact angle to improve surface wetting of the TS-MMS materials. Each data point represents n=5 images collected from separate films using deionized water as the solvent. Contact angles were measured using a Ramd-Hart 200-F1 goniometer.

Impact on Mechanical Properties of the TS-MMS Films Having PLGA

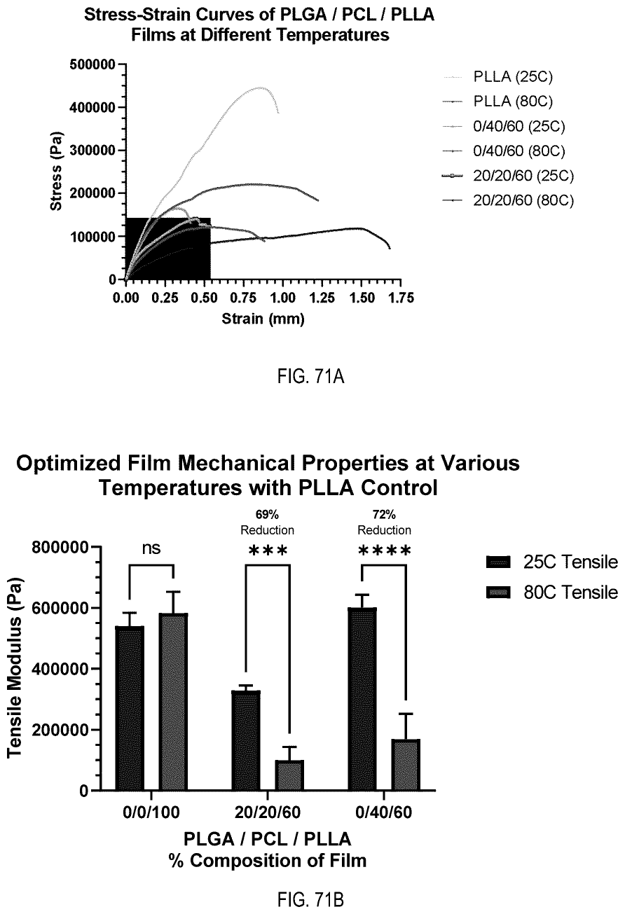

The stress-strain curves and tensile strength of different TS-MMS films having PLGA were tested, and the test results are shown in FIGS. 71A and 71B respectively. The test results in FIGS. 71A and 71B demonstrated that introduction of PLGA-DA caused a decrease in tensile modulus at room temperature, but maintained thermosensitive changes in tensile strength similarly to 40/60 material. PLGA-DA enables a greater strain at yield, indicating a more elastic material which may be beneficial for some applications.

Impact on Shape Memory Recovery Time of TS-MMS Films Having PLGA

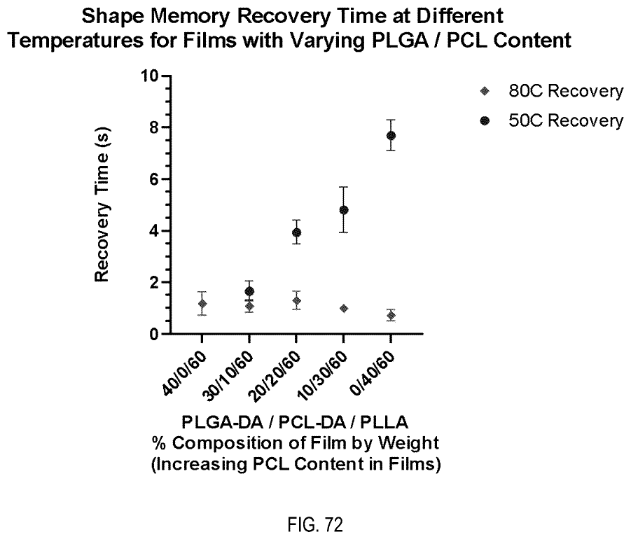

The shape memory recovery time of different TS-MMS films having PLGA were tested, and the test results are shown in FIG. 72. The test results in FIG. 72 demonstrated that the introduction of PLGA-DA enabled shape memory properties at 50° C. requiring a minimum of 10% PCL-DA. The TS-MMS film having PLGA/PCL/PLLA in a weight ratio of 40/0/60 did not have shape memory properties, as shown in FIG. 72.

Impact on Controlled Release Kinetics of TS-MMS Films Having PLGA

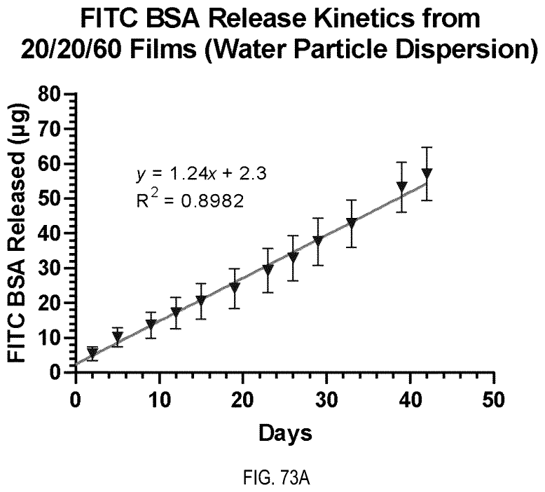

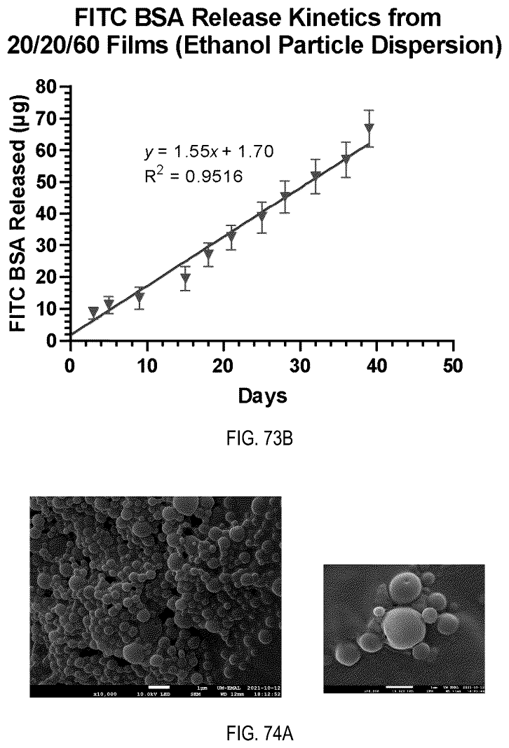

The TS-MMS membranes having PLGA-DA/PCL-DA/PLLA (20/20/60) are capable of controlled release of protein therapeutics (Slide 30) as well as small molecule (29) demonstrated here. Either water or ethanol may be used to disperse particles in the mold prior to film casting (pics are in the original disclosure+description of method). The FITC BSA release kinetics from TS-MMS membranes having PLGA-DA/PCL-DA/PLLA (20/20/60) with water particle dispersion and ethanol particle dispersion are shown in FIGS. 73A and 73B, respectively.

Directional Release of Embedded Drug Substance TS-MMS Membranes

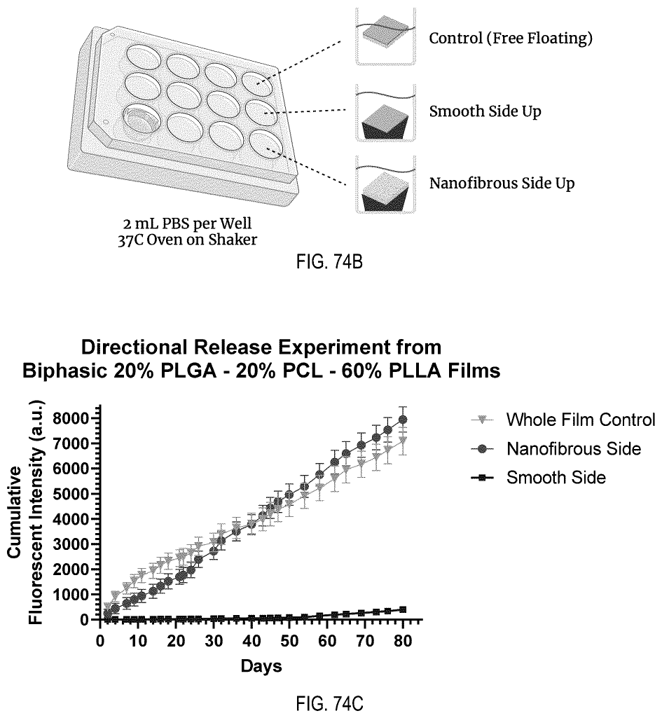

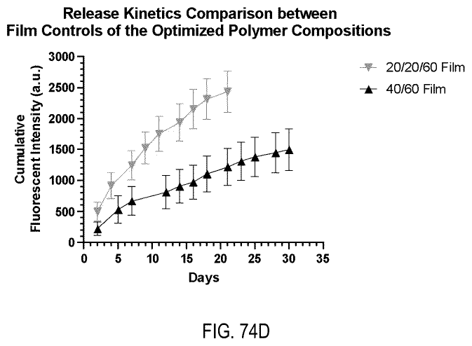

Embedded control release system in the macroporous scaffold were prepared in Example 16. The nanoparticles with the model drug were embedded into the nanofibers of TS-MMS at the time of fabrication. The nanoparticles are shown by SEM in FIG. 74A. An experiment was designed to determine the directional release of drug from the bilayer TS-MMS membrane, as shown in FIG. 74B. As shown in FIG. 67 (or FIGS. 48B-48C and 49A-49B in Example 15) the nanoparticles was localized to the nanofiber region, and not the smooth region. In this study, the TS-MMS membranes loaded with nanoparticles containing a model drug were put into a resin leaving one surface exposed, either the smooth or nanofibrous surface, compared to non-invested controls, which were incubated in PBS at 37° C. on a shaker, as shown in FIG. 74B. The test results in FIG. 74C demonstrated that the majority of drug release came from the nanofibrous side, indicating directional release from the regenerative side of the membrane into the defect compartment. A negligible controlled release from the smooth side was only detected at late time points, and was likely the result of degradation and increasing porosity of this smooth layer. The test results in FIG. 74D demonstrated that incorporation of PLGA-DA as a substitute for part of the PCL-DA component results in a more rapid release kinetics.

Impact on Degradation of TS-MMS Membranes Having PLGA

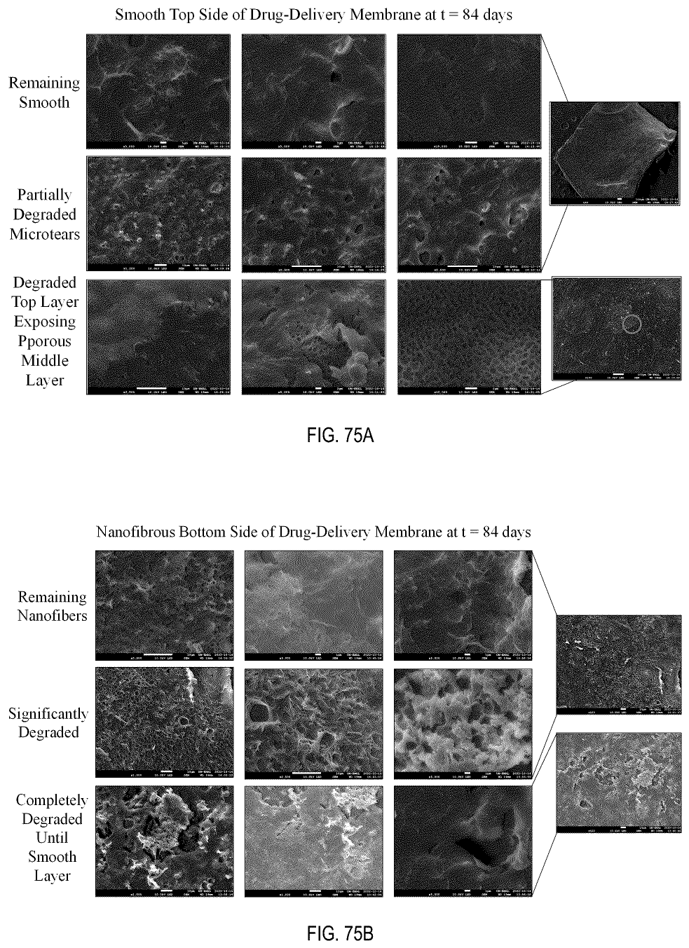

The degradation rate of the TS-MMS membranes were tested using SEM images. The SEM micrographs of TS-MMS membranes after 84 days of degradation (PBS, 37° C., shaker) for both the smooth top side and nanofibrous bottom side were tested and shown in FIGS. 75A and 75B, respectively. The test results in FIGS. 75A and 75B demonstrated that the nanofibrous side (porous bottom side) degraded much more quickly than the smooth side (with majority PCL). The test results agree with previous TGA and degradation experiment, as shown in FIGS. 38A-44 in Example 13.

Impact on Crystallinity of TS-MMS Membranes Having PLGA

The impact of the PLGA on the crystallinity of the TS-MMS membranes having PLGA was studied, and the test results are shown in FIGS. 76A and 76B. The PLLA nanofibers are an advantageous design motif because they facilitate protein adsorption and cell adhesion to the construct, critical for regeneration and tissue integration. The experimental tests were conducted to determine the minimum wt/wt % of PLLA by weight of the total polymers necessary in the TS-MMS compositions (made from PLLA and PCL-DA; and PLLA and PLGA-DA, respectively) of the present disclosure necessary to maintain the nanofibrous architecture of PLLA.

Similar to the scaffolds (PLLA/PCL in a weight ratio of 60:40) tested, the fabrication of the membrane maintained the crystallinity, and a minimum amount of the PLLA was required for forming the phased separated PLLA crystalline nanostructures was about 50%. In this study, the PLLA used had a molecular weight higher than 50 kDa MW. This minimum amount of PLLA is critical for PLLA nanofiber formation. The XRD signature of PLLA is maintained in TS-MMS membranes having PLGA.

### Example 20

In-Vivo Subcutaneous Implantation

This study is to determine the effect of bilayer morphology, pore deformation and recovery on tissue integration, cell infiltration and host response.

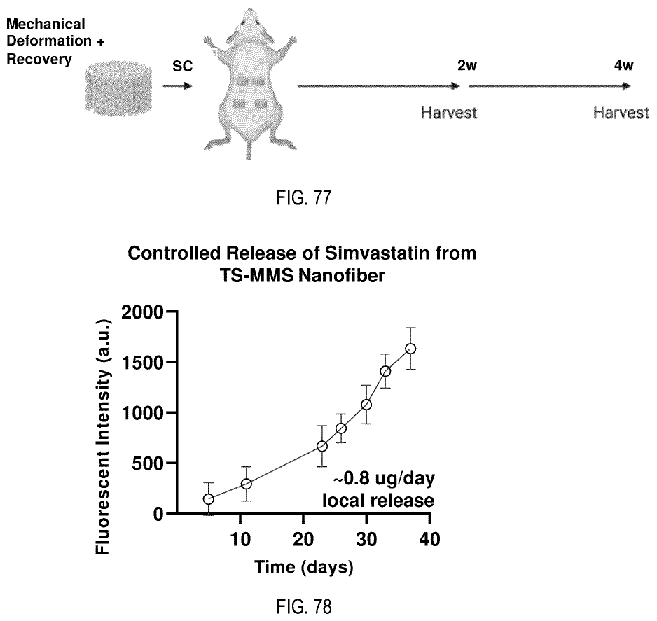

Experimental Design: The macroporous scaffolds were fabricated, deformed, and recovered as described herein. The scaffolds tested were pure PLLA scaffolds (control); the TS-MMS scaffolds (prepared in Example 6); and TS-MMS scaffolds having simvastatin controlled release particles embedded in the scaffold (prepared in Example 16). The macroporous scaffolds were sterilized by ethylene oxide, and then in 70% ethanol and washed 3 times with sterile PBS prior to implantation into healthy adult male mice (12 weeks old, ColleGFP; LysMCre; Td following 3 times tamoxifen induction). The mice were anesthetized by isoflurane and administered prophylactic carprofen for pain management. After surgical scrub, a 2 cm mid-dorsal incision was made and four pockets were created by blunt dissection. One acellular scaffold construct was implanted into each pocket. The incision was closed by suturing. The animals were sacrificed by CO2 euthanasia and bilateral pneumothorax at 2 weeks and 4 weeks post-surgery. The experimental process is shown in FIG. 77. The cell-scaffold constructs were explanted by blunt dissection and fixed in 4% PFA overnight. The samples were prepared for cryosectioning and paraffin sectioning.

TS-MMS membranes and scaffold were able to be sutured to retain them within a tissue or defect site, and demonstrated a decreased suture pull out strength at a high temperature—higher than the glass transition temperature and melting temperature of one of the polymers in the TS-MMS membrane and scaffold—making them easy to suture into the defect sites. However, suture pullout strength increased at 37° C., compared to the temperature higher than the melting point of one of the polymers, which was favorable to maintain the suture and resist its pulling out at or near physiologic temperature of about 37° C. and 42° C.

The controlled release of simvastatin from the TS-MMS scaffold was studied, and the test results are shown in FIG. 78.

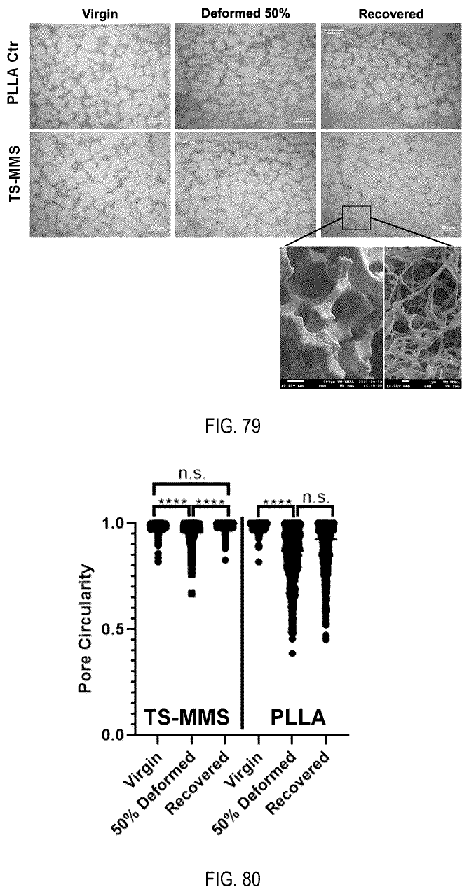

The macropore recovery in the TS-MMS macroporous scaffold was studied using the same method described in Example 8, and the SEM images of the control and test samples were used to show the pore recovery after deformation, as shown in FIG. 79. The images of histologic sections (n>100 sections per sample) were taken with a stereomicroscope and analyzed by measuring pore circularity as shown in FIG. 80. The testing results demonstrated that the TS-MMS scaffold of the disclosure showed nearly full recovery of the macropores after deformation, as shown in FIG. 79 (bottom), while the macropores of the pure PLLA scaffold control sample were significantly flattened and insignificantly changed after recovery at 55° C., as shown in FIG. 79 (top). As demonstrated below, these pores facilitate robust vascularization, cell and tissue infiltration towards accelerated and predictable healing owning to their enhanced clinical handling properties as compared to the PLLA control sample.

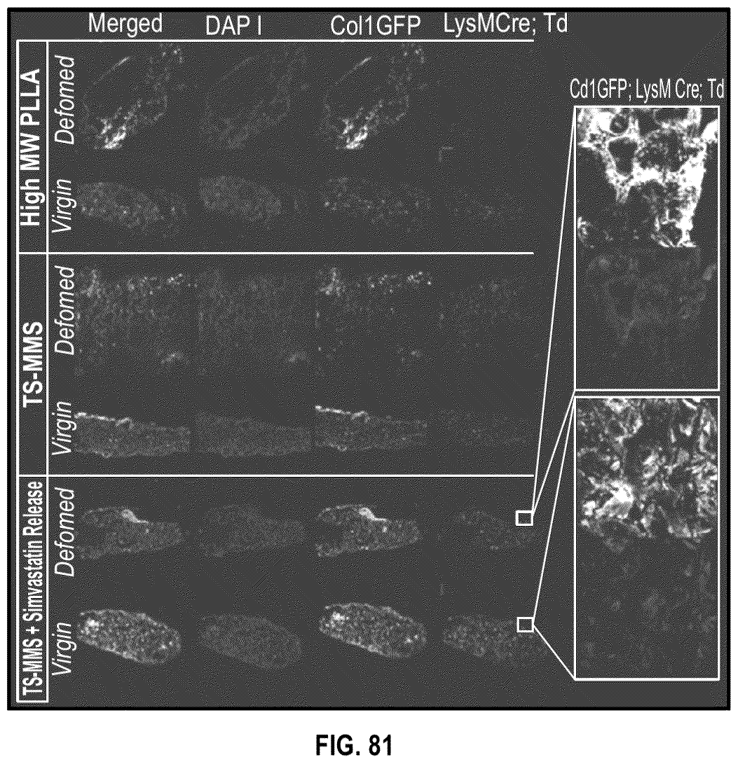

H&E: The H&E stain showed that occluded pores which did not recover (pure PLLA, deformed) did not allow for cell and tissue infiltration even after four weeks of time, as shown in FIG. 81 (top, control). The occluded pores led to fibrous tissue encapsulation which is not favorable for facilitating regeneration. In contrast, the TS-MMS scaffolds (PLLA/PCL-DA (60/40 composition) without (FIG. 81, middle) and with (FIG. 81, bottom) simvastatin controlled release particles recovered their patent macropores after deformation and facilitated robust cellularization in as little as two weeks, insignificantly different than the virgin (non-deformed) scaffold control, as shown in FIG. 81 (middle) and FIG. 81 (bottom), respectively. The images were from paraffin sections and stained according to standard protocols, imaged by light microscope, as shown in FIG. 81 for the pure PLLA scaffold (top, control), TS-MMS scaffold (PLLA/PCL-DA) (middle), and TS-MMS scaffold having simvastatin controlled release particles (bottom), respectively.

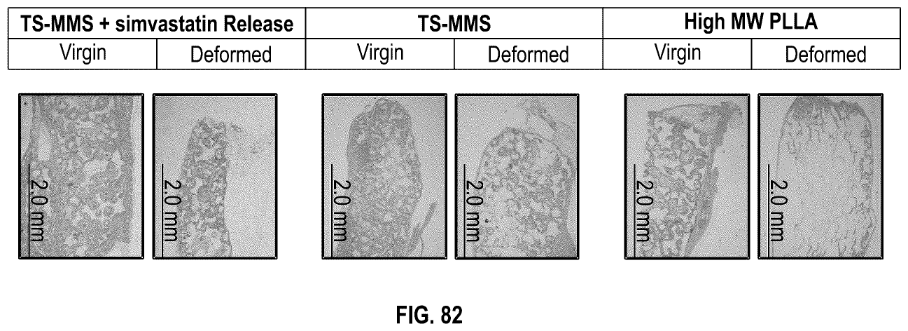

The cell migration and infiltration was determined by H&E staining and observation with a light microscope, as shown in FIG. 82 for the pure PLLA scaffold (top, control), TS-MMS scaffold (PLLA/PCL-DA) (middle), and TS-MMS scaffold having simvastatin (bottom), respectively. As shown in FIG. 82, after 4 weeks of implantation, there was not much pink that was observed on the image of the deformed pure PLLA scaffold (top), indicating that there was little to no cells infiltration into the pure PLLA scaffold (top). In contrast, significantly amounts of pink color were observed on the images of the TS-MMS scaffold (middle) and TS-MMS having simvastatin scaffold (bottom), respectively. This is because, the pores of both the TS-MMS scaffolds recovered after scaffold deformation, and the pore recovery enabled the cell infiltration, tissue integration and maturation, as shown in FIG. 82 (middle) and FIG. 82 (bottom), respectively.

Fluorescence: Col1eGFP is a marker of bone regeneration; LysMCre; Td marks cells from the hematopoetic lineage. As shown in FIG. 81 (under LysMCre; Td column), the virgin (100%) PLLA (top) and virgin TS-MMS scaffolds (middle and bottom) facilitated similar levels of cell infiltration (well cellularized even into the center of the scaffold) and some infiltration of hematopoetic cells (monocytes and osteoclasts). In contrast, the deformed PLLA (top) facilitated little to no cell infiltration. The controlled release of simvastatin, an angiogenic agent, from the nanoparticles attached to the nanofibers of the first polymer (NF-NP) of the TS-MMS (middle and bottom) facilitated robust cell infiltration and osteoclast honing, indicative of engineered tissue maturation. The sections were from cryopreserved sections, counterstained with DAPI, imaged by confocal microscope. The cell infiltration and migration was determined by DAPI signal which marked the cell nuclei.

CD31: The PLLA scaffolds with deformed pores led to an avascular, acellular scaffold at 2 and 4 weeks. On the other hand, the TS-MMS scaffolds with deformed pores enabled robust vascularization comparable to virgin TS-MMS or PLLA scaffolds at the same time points. The simvastatin increased the number and size of CD31+ blood vessels which demonstrated the efficacy of the controlled release platform in the TS-MMS material. The sections were from the paraffin embedding, stained by standard immunohistochemistry method and CD31 primary antibody (rabbit anti-mouse) using DAB immunogen, and imaged by light microscope.

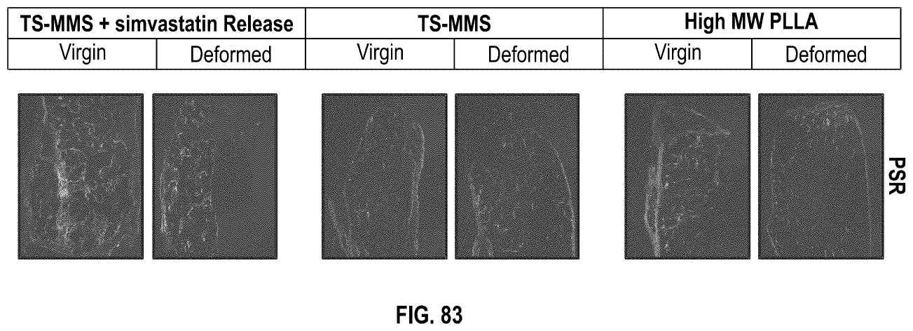

Picrosirius red (PSR) staining viewed under polarized light microscopy is the standard method to evaluate the organization of collagen fibers in tissues. Increased signal corresponds to increased collagen secretion and maturation by cells which migrate into the scaffold. In essence, these cells which migrate into the artificial environment. The PSR images are shown in FIG. 83. As shown in FIG. 83, after 4 weeks of implantation, the PSR image of the deformed pure PLLA scaffold (top) was observed to be dark without much red color, indicating that there was little to no cell infiltration into the pure PLLA scaffold (top). In contrast, significant amounts of red color were observed on the images of the TS-MMS scaffold (middle) and TS-MMS having simvastatin scaffold (bottom), respectively. The PSR images further demonstrated that the pores of both the TS-MMS scaffolds with (FIG. 83, bottom) and without (FIG. 83, middle) simvastatin recovered after scaffold deformation, and the pore recovery enabled the cell infiltration, tissue integration and maturation, as shown in FIG. 83 (middle) and FIG. 83 (bottom), respectively. Further, the simvastatin controlled release system further augmented vascularization and increased tissue maturation, as shown in FIGS. 81-83 (bottom).

In Vivo Validation on Periodontal Defect

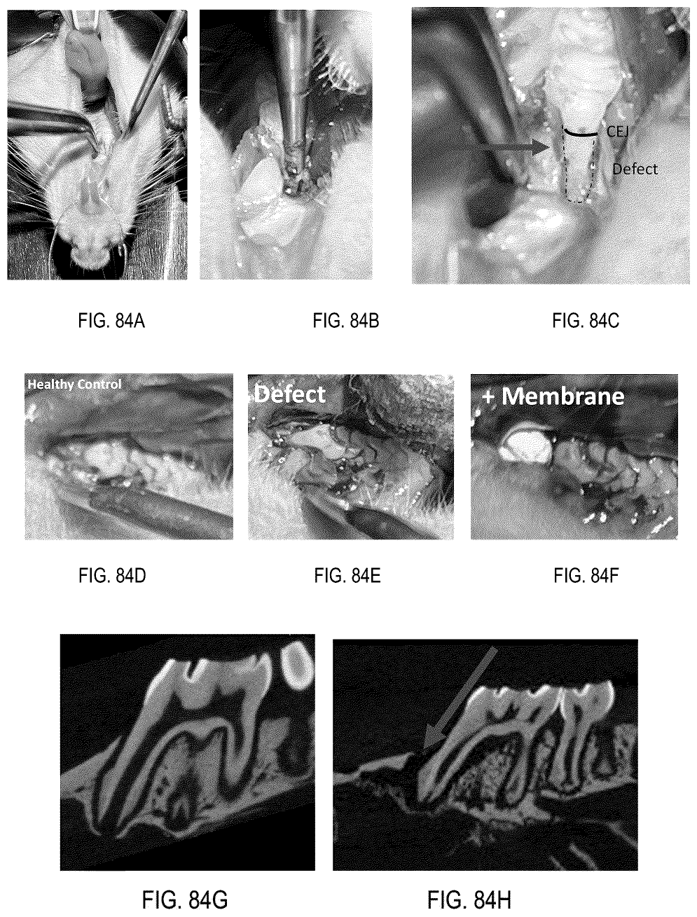

In vivo functionality of TS-MMS membranes to facilitate periodontal regeneration was evaluated in a periodontal defect model in rats. Virgin male and female Sprague-Dawley rats (3 months old) were used for testing membranes in vivo under an approved animal protocol. The rats were anesthetized by ketamine-xylazine and prepared with a preoperative scrub and prophylactic carprofen administration. A full thickness mucoperiosteal flap was elevated from a midcrestal incision to uncover alveolar bone adjacent to the maxillary first molars, under a microscope, as shown in FIG. 84A. A #2 carbide round bur and slow speed handpiece, with copious irrigation, was used to remove the alveolar bone covering the tooth's mesial root surface to create the defects, as shown in FIGS. 84B and 84C. FIGS. 84D and 84E show the healthy control rat with incision but no bone defects and the rats with defects, respectively. After the defect was created (1.5 mm×3 mm×1 mm), a membrane was implanted into the defect location and in contact with the periosteum at the margins of the defect, as shown in FIG. 84F. The flap was repositioned, and incision was closed with cyanoacrylate; and the rats were able to recover. FIGS. 84G and 84H show 2D radiograph (reconstructed from uCT) of the defects at the mesial root in FIG. 84E for the healthy control rat and the rats with the defects, respectively. A widened PDL space was observed (labelled with arrow) for the defects at the mesial root, as shown in FIG. 84H. FIG. 84I shows a 3D reconstruction of the same defect shown in FIG. 84E

Experimental Set Up: Different groups of rats were tested in this study. Group 1 were healthy rats with incision surgery but without defects and without treatment which were tested as healthy controls. Group 2 were the rats with both the incision surgery and bone defect but no treatment which were tested as negative controls. The rats with bone defects that were treated with different membranes which included TS-MMS membrane, TS-MMS membrane having PDGF, and GuideOR control membrane, were set up as Groups 3, 4 and 5 respectively. The GuideOR control membrane is a commercially available PDLA membrane and is FDA approved. The sample size (n) was set as per time point per group (16 rats, 32 defects—contralateral).

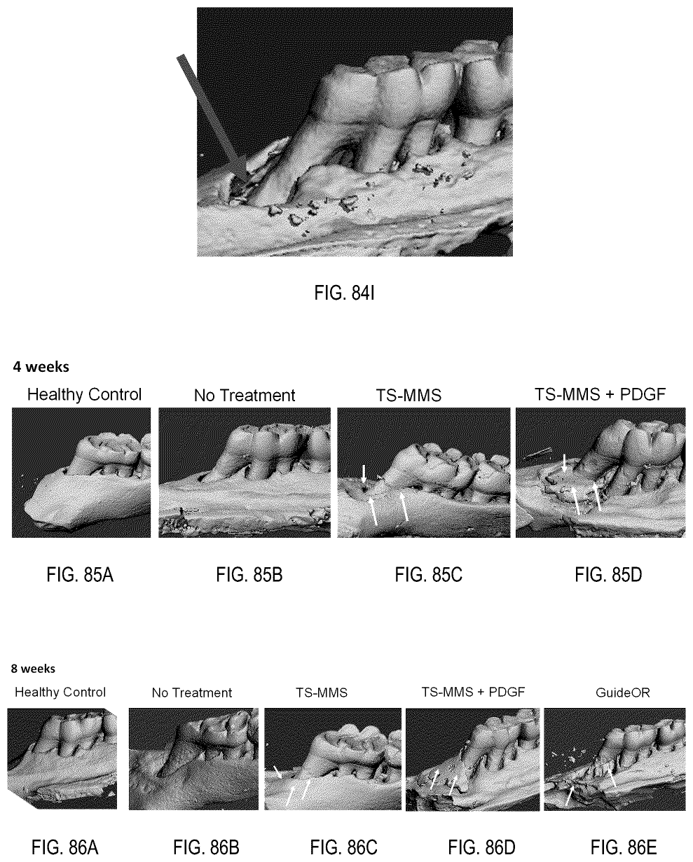

After 4 and 8 weeks, the rats were sacrificed and the recovered bone at the defect site was evaluated by microcomputed tomography analysis. The 3D reconstruction of the same defects for these rats with different treatments after 4 weeks and 8 weeks were made based on the microcomputed tomography analysis results, and the images of the 3D reconstructions are shown in FIGS. 85A-D and FIGS. 86A-86E, respectively. The new bone formation was marked in arrows in these figures.

The test results as shown in FIGS. 85A-D and FIGS. 86A-86E clearly demonstrated that the new bone formation for the rats treated with TS-MMS membrane having PDGF was the best at both 4 weeks and 8 weeks test points, followed by the rats treated with TS-MMS membrane, and then the rats treated with GuideOR control. No new bone formation was observed for the rat having no treatment at either 4 or 8 weeks test point, as shown in FIGS. 85B and 86B, respectively.

Benefits of the current technology of the present disclosure include: a) a novel tissue engineering matrix with improved clinical handling properties, designed for periodontal and craniofacial regeneration applications; b) these matrices are fabricated as surgical membranes which serve not only as a protective barrier but also to induce regeneration through directional controlled release of inductive substances, facilitate regeneration through their tissue integration, and define and maintain dimensional stability in horizontal and/or vertical defects; c) these matrices are fabricated as macroporous scaffolds and the novel chemistry serves to deliver a three-dimensional environment capable of facilitating tissue ingrowth, vascularization, extracellular matrix deposition and remodeling, and tissue regeneration; d) a novel approach to specifically deliver one or more drug to the defective sits with controlled release rates and profiles; and/or e) easy manufacturing process and lower manufacturing cost. Example embodiments are provided so that this disclosure will be thorough, and will fully convey the scope to those who are skilled in the art.

Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

The following non-limiting discussion of terminology is provided with respect to the present technology. The headings (such as “Introduction” and “Summary”) and sub-headings used herein are intended only for general organization of topics within the present disclosure and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the term “bulk crystallinity” may refer to a degree of crystallinity in the bulk of the polymeric material. As used herein, the term “surface crystallinity” may refer to a degree of crystallinity in the surface of the polymeric material.

As used herein, the term “biodegradable” refers to the ability of a polymer, composition, or material to erode or degrade in vivo to form smaller chemical fragments.

As used herein, the term “biocompatible material” refers to any material that does not deteriorate appreciably and does not induce a significant immune response or deleterious tissue reaction, e.g., toxic reaction or significant irritation, over time when implanted into or placed adjacent to the biological tissue of a subject or induce blood clotting or coagulation when it comes in contact with blood.

As used herein, the term “sugar sphere” refers to a sugar particle having a substantially spherical form. A substantially spherical sugar particle is a sugar particle with a difference between the smallest radii and the largest radii generally not greater than about 40% of the smaller radii, and more typically less than about 30%, or less than 20%.

As used herein, the term “microsphere” refers to a microparticle having a substantially spherical form. A substantially spherical microparticle is a microparticle with a difference between the smallest radii and the largest radii generally not greater than about 40% of the smaller radii, and more typically less than about 30%, or less than 20%.

As used herein, the term “ultraviolet (UV) light” refers to electromagnetic radiation with a wavelength shorter than that of visible light, or from about 10 nm to about 420 nm. In embodiments, sub-ranges of ultraviolet light may be used, for example from about 100 to about 400 nm, from about 200 to about 400 nm, from about 250 to about 400 nm, from about 250 to about 365 nm, from about 300 to about 400 nm, or from about 350 to about 400 nm.

As used herein, the words “desire” or “desirable” refer to embodiments of the technology that afford certain benefits, under certain circumstances. However, other embodiments may also be desirable, under the same or other circumstances. Furthermore, the recitation of one or more desired embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of nonrestrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

“A” and “an” as used herein indicate “at least one” of the item is present; a plurality of such items may be present, when possible.

“About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. As used herein, the term “about” when used in connection with a value may refer to ±10% variation from the value. Other than in the operating examples, or where otherwise indicated, all numbers expressing quantities of ingredients or reaction conditions used herein should be understood as modified in all instances by the term “about.”

As used herein, “about,” “approximately,” “essentially” and “substantially” are understood to refer to numbers in a range of numerals, for example the range of −10% to +10% of the referenced number, or −5% to +5% of the referenced number, or −1% to +1% of the referenced number, or −0.1% to +0.1% of the referenced number.

As used herein, the term “substantially no,” “substantially not,” “essentially free” or “substantially free” as used in reference to a particular component may mean that any of the component present constitutes less than 10% by weight, such as less than 9%, less than 8%, less than 7%, less than 6%, less than 5%, less than 4%, less than 3%, less than 2%, less than 1%, less than 0.5% or less than 0.1% by weight.

When an element or layer is referred to as being “on,” “engaged to,” “connected to” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The abbreviation, “e.g.” or “i.e.” are used herein to indicate a non-limiting example. Thus, the abbreviation “e.g.” or “i.e.” is synonymous with the term “for example.” Where used herein, the terms “example” and “such as,” particularly when followed by a listing of terms, are merely exemplary and illustrative and should not be deemed to be exclusive or comprehensive.

As used herein, the term a “subject” or “individual” is a mammal, or a human.

1. A method of making a biodegradable polymeric material, the method comprising:
reacting:
a polymer mixture comprising a first polymer and a second polymer, the first polymer being a biodegradable linear polymer and having first polymer chains, the second polymer being a biodegradable polymer having second polymer chains each of which has two or more functional groups, the first and second polymers being present in a weight ratio of between about 50:50 and about 99:1; and
a reagent to form coupled second polymer chains in presence of the first polymer chains, thereby forming the biodegradable polymeric material,
wherein the reagent is selected from a group of a radical initiator, a catalyst, a crosslinking agent, or a combination thereof, and
wherein the biodegradable polymeric material has an interpenetrating polymer network (IPN) between the first and second polymer chains, wherein the reacted second polymer chains are interspersed throughout the first polymer chains.

2.-42. (canceled)

43. A biodegradable polymeric material comprising:
a first polymer and a second polymer provided in a weight ratio in a range of about 50:50 and about 99:1,
wherein the first polymer is a biodegradable linear polymer having first polymer chains and a crystallinity of about 20-100%,
wherein the second polymer is a biodegradable polymer having second polymer chains each of which has two or more functional groups, and at least a portion of the second polymer chains are coupled to each other through the functional groups, and
wherein the biodegradable polymeric material has an interpenetrating polymer network (IPN) between the first polymer chains and the second polymer chains, wherein the second polymer chains are interspersed throughout the first polymer chains.

44.-55. (canceled)

56. A method of making a biodegradable surgical membrane, the method comprising:
admixing
a polymer mixture comprising a first polymer and a second polymer in a weight ratio in a range of about 50:50 and about 99:1, the first polymer having first polymer chains and being a biodegradable linear polymer, the second polymer being a biodegradable polymer having second polymer chains each of which have two or more functional groups; and
a first organic solvent to form a polymer mixture solution having an interpenetrating polymer network (IPN) between the first and second polymer chains;
admixing a reagent solution with the polymer mixture solution to form a polymer-reagent solution, wherein the reagent is selected from a group of a radical initiator, a catalyst, a crosslinking agent, or a combination thereof; and
reacting the polymer-reagent solution to form coupled second polymer chains in presence of the first polymer chains, thereby forming the biodegradable surgical membrane,
wherein the polymer mixture solution comprises about 1-30 wt. % of the polymer mixture and about 70-99 wt. % of the first organic solvent, by weight of the polymer mixture solution.

57.-110. (canceled)

111. A biodegradable surgical membrane comprising:
a first polymer and a second polymer in a weight ratio of between about 50:50 and about 99:1, the first polymer being a biodegradable linear polymer having first polymer chains and a crystallinity of about 20-80%, the second polymer being a biodegradable polymer having second polymer chains each of which has two or more functional groups, at least a portion of the second polymer chains being coupled with each other through the two or more functional groups,
wherein the biodegradable surgical membrane has an interpenetrating polymer network (IPN) between the first polymer chains and the second polymer chains,
wherein the second polymer chains are interspersed throughout the first polymer chains,
wherein the biodegradable surgical membrane has a biphasic morphology including a top smooth layer having a porosity less than about 40% v/v, and a bottom porous layer having a porosity higher than about 60% v/v.

112. The biodegradable surgical membrane of claim 111, wherein the biodegradable surgical membrane has a thickness of 0.1-50 mm, the top smooth layer has a thickness of 0.01-10 mm, and the bottom porous layer has a thickness of 0.1-50 mm.

113. The biodegradable surgical membrane of claim 111, wherein the bottom porous layer has pores having an average pore size of about 0.1-100 μm.

114. The biodegradable surgical membrane of claim 111, wherein a degradation rate of the bottom porous layer is about 1-10 times faster than a degradation rate of the top smooth layer.

115. The biodegradable surgical membrane of claim 111, further comprising a third polymer having third polymer chains each of which has two or more functional groups that can react with the two or more functional groups of the second polymer chains to couple the third polymer chains to the second polymer chains, wherein the reacting comprises admixing the second polymer and the third polymer to thereby:
(i) couple (a) at least a portion of the second polymer chains to each other; (b) least a portion of the second polymer chains to at least a portion of the third polymer chains; and/or (c) at least a portion of the third polymer chains to each other, to thereby chain extend at least a portion of the second polymer chains and/or third polymer chains; and/or
(ii) partially crosslink (a) at least a portion of the second polymer chains to each other; (b) at least a portion of the second polymer chains to at least a portion of the third polymer chains; and/or (c) at least a portion of the third polymer chains to each other,
wherein the biodegradable surgical membrane comprises an IPN of the first, second and third polymer chains, and
wherein the second and third polymer chains are interspersed throughout the first polymer chains.

116. The biodegradable surgical membrane of claim 111, further comprising a compound having two or more functional groups that can react with the two or more functional groups of the second polymer chains to couple at least a portion of the second polymer chains by coupling one or more second polymer chains with the compound and form coupled second polymer chains including extended and/or at least partially crosslinked second polymer chains,
wherein the biodegradable surgical membrane comprises an IPN of the first polymer chains and the coupled second polymer chains, and
wherein the coupled second polymer chains are interspersed throughout the first polymer chains

117. The biodegradable surgical membrane of claim 111, wherein the second polymer has a crosslinking density of 0.1-30%.

118. The biodegradable surgical membrane of claim 115, wherein the second and third polymers have a crosslinking density of 0.1-30%.

119. The biodegradable surgical membrane of claim 111, wherein the biodegradable surgical membrane is a periodontal membrane having a thickness of about 0.1-50 mm.

120. A method for providing a dental implant to a subject in need thereof, the method comprising:
implanting a periodontal membrane comprising the biodegradable surgical membrane of claim 111 to a defective dental site of a subject.

121.-126. (canceled)

127. A method for making a macroporous tissue engineering scaffold, the method comprising:
admixing
a polymer mixture comprising a first polymer and a second polymer in a weight ratio between about 50:50 and about 99:1, the first polymer being a biodegradable linear polymer and having first polymer chains, the second polymer being a biodegradable polymer and having second polymer chains each of which comprises two or more functional groups; and
a first organic solvent to form a polymer mixture solution having an interpenetrating polymer network (IPN) between the first and second polymer chains,
wherein the second polymer chains are interspersed throughout the first polymer chains,
wherein the polymer mixture solution comprises about 1-30 wt. % of the polymer mixture by weight of the polymer mixture solution and about 70-99 wt. % of the first organic solvent by weight of the polymer mixture solution;
admixing a reagent solution with the polymer mixture solution to form a polymer-reagent solution, wherein the reagent is selected from a group of a radical initiator, a catalyst, a crosslinking agent, or a combination thereof;
combining in a container the polymer-reagent solution and a sugar porogen scaffold template comprising sugar particles;
reacting the polymer-reagent solution to form coupled second polymer chains in presence of the first polymer chains, thereby forming an initial scaffold including the sugar sphere porogen scaffold template;
cooling the initial scaffold; and
dissolving the sugar spheres in the initial scaffold thereby forming the macroporous tissue engineering scaffold,
wherein the macroporous tissue engineering scaffold has an IPN between the first and coupled second polymer chains, and the coupled second polymer chains are interspersed throughout the first polymer chains.

128.-165. (canceled)

166. A method for providing a dental implant to a subject in need thereof, the method comprising:
implanting the macroporous tissue engineering scaffold made by the method of claim 127 to a defective dental site in the subject.

167.-177. (canceled)

178. A macroporous tissue engineering scaffold comprising:
a first polymer and a second polymer in a weight ratio of between about 50:50 and about 99:1,
the first polymer being a biodegradable linear polymer and having first polymer chains,
the second polymer being a biodegradable polymer, the second polymer having second polymer chains each of which has two or more functional groups, the second polymer being coupled with each other through the two or more functional groups,
wherein the macroporous tissue engineering scaffold has an interpenetrating polymer network (IPN) between the first polymer chains and the second polymer chains, and the second polymer chains are interspersed throughout the first polymer chains,
wherein the first polymer is at least partially crystallized and at least partially phase separated from the second polymer, the first polymer has a crystallinity of about 20-80%, and the first polymer having crystalline nanostructures homogeneously dispersed in the IPN, and
wherein the macroporous tissue engineering scaffold has a porosity of about 90-99% v/v and has macro-pores having an average pore size of about 30-450 μm.

179.-187. (canceled)

188. A method for providing a dental implant to a subject in need thereof, the method comprising:
implanting the macroporous tissue engineering scaffold of claim 178 to a defective dental site in the subject.

189. (canceled)

190. A method of preparing a macroporous tissue engineering scaffold comprising an embedded controlled release system, the method comprising:
admixing
a polymer mixture comprising a first polymer and a second polymer in a weight ratio in a range between about 50:50 and about 99:1, the first polymer being a biodegradable linear polymer and having first polymer chains, the second polymer being a biodegradable polymer and having second polymer chains each of which comprises two or more functional groups; and
a first organic solvent to form a polymer mixture solution having an interpenetrating polymer network (IPN) between the first and second polymers, wherein the polymer mixture solution comprises 1-30 wt. % of the first and second polymers and 70-99 wt. % of the first organic solvent by weight of the polymer mixture solution;
admixing a reagent solution with the polymer mixture solution in a volume ratio of 0.1:100 to 30:100 v/v to form a polymer-reagent solution, the reagent solution comprising a reagent and a second organic solvent, wherein the reagent is selected from a group of a radical initiator, a catalyst, a crosslinking agent, or a combination thereof;
combining in a container the polymer-reagent solution and a sugar porogen scaffold template, the sugar porogen scaffold template comprising sugar particles and nanoparticles, and the nanoparticles attached to a surface of the sugar particles; and
reacting the polymer-reagent solution in the container to form coupled second polymer chains in presence of the first polymer chains, thereby forming an initial scaffold including the sugar sphere porogen scaffold template,
cooling the initial scaffold; and
dissolving the sugar particles in the initial scaffold thereby forming the macroporous tissue engineering scaffold, wherein the dissolving does not remove the nanoparticles from the initial scaffold, and the nanoparticles are retained and dispersed in the macroporous tissue engineering scaffold,
wherein the macroporous tissue engineering scaffold has an IPN between the first and coupled second polymer chains, and the second polymer chains are interspersed throughout the first polymer chains.

191.-213. (canceled)

214. A system having a controlled release composition embedded in a macroporous tissue engineering scaffold, the system comprising:
the macroporous tissue engineering scaffold comprising:
a first polymer and a second polymer in a weight ratio of between about 50:50 and about 99:1,
the first polymer being a biodegradable linear polymer having first polymer chains, the second polymer being a biodegradable polymer,
the second polymer having second polymer chains each of which has two or more functional groups, the second polymer being chain extended and/or at least partially crosslinked with each other through the two or more functional groups,
wherein the macroporous tissue engineering scaffold has an interpenetrating polymer network (IPN) between the first and second polymer chains, wherein the second polymer chains are interspersed throughout the first polymer chains, and
wherein the first polymer is at least partially crystallized and at least partially phase separated from the second polymer, the first polymer has a crystallinity of about 20-80%, and the first polymer having crystalline nanostructures homogeneously dispersed in the IPN; and
the controlled release composition comprising:
first nanoparticles comprising a fourth polymer and a first drug substance homogeneously dispersed in the fourth polymer,
wherein the first nanoparticles are dispersed in the macroporous tissue engineering scaffold, the first nanoparticles have an average particle size of about 10-1000 nm, and
wherein the macroporous tissue engineering scaffold having the controlled release composition has a porosity of about 90-99% v/v and has macro-pores having an average pore size of about 30-450 μm.

215. (canceled)

221. The system of any one of claim 214, wherein the system is provided as a dental implant.

222. A method for providing a dental implant to a subject in need thereof, the method comprising:
implanting the system of claim 221 to a defective dental site in the subject.

223.-232. (canceled)
